(19) United States
(12) Patent Application Publication
(10) Pub. No.: US 2025/0313554 A1
(43) Pub. Date: Oct. 9, 2025

(54) PTPN2 INHIBITORS

(71) Applicant: Ness Therapeutics, Inc., San Diego, CA (US)

(72) Inventors: Ruben Abagyan, La Jolla, CA (US); Volodymyr Kysil, San Diego, CA (US); Vladislav Zenonovich Parchinsky, Moscow (RU); Alexei Pushechnikov, San Diego, CA (US); Alexandre Vasilievich Ivachtchenko, Hallandale Beach, FL (US); Nikolay Savchuk, San Diego, CA (US)

(21) Appl. No.: 18/865,333

(22) PCT Filed: May 8, 2023
(86) PCT No.: PCT/US2023/066748

§ 371 Date: Nov. 12, 2024

Related U.S. Application Data
(60) Provisional application No. 63/341,908, filed on May 13, 2022.

(51) Int. Cl. C07D 417/12; A61K 31/506; A61K 31/517; A61P 35/00; C07D 417/14

(52) U.S. Cl. CPC C07D 417/12; A61K 31/506; A61K 31/517; A61P 35/00; C07D 417/14

(57) ABSTRACT

The present invention is generally directed to inhibitors of protein tyrosine phosphatase enzymes (PTPN1 and/or PTPN2) useful in the treatment of diseases and disorders modulated by said enzymes and having the Formula (I):

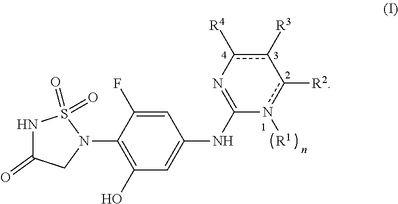

### CROSS-REFERENCE TO RELATED APPLICATIONS

This application claims priority to and benefit of U.S. Provisional Patent Application Ser. No. 63/341,908 filed May 13, 2022, entitled “PTPN2 Inhibitors”, the disclosure of which is incorporated by reference in its entirety for all purposes.

### FIELD OF INVENTION

The present invention is directed to inhibitors of PTPN1/PTPN2. The inhibitors described herein can be useful in the treatment of diseases or disorders associated with Protein Tyrosine Phosphatase Non-Receptor Type 1 (PTPN1) or/and Protein Tyrosine Phosphatase Non-Receptor Type 2 (PTPN2), such as Endocrine diseases, Genetic diseases, Immune diseases, Metabolic diseases, Bone diseases, Eye diseases, Respiratory diseases, Gastrointestinal diseases, Infectious diseases, Blood diseases, Cancer diseases. In particular, the invention is concerned with compounds and pharmaceutical compositions that inhibit the PTPN1/PTPN2, methods of treating diseases or disorders associated with PTPN1/PTPN2, and methods of synthesizing these compounds.

### BACKGROUND

A main immune system function is the protection of the human body against the occurrence of malignancy by eliminating damaged, altered, or aged cells. The DNA in many mutated cancer cells produce abnormal proteins known as tumor antigens, which marks them as altered or damaged. The immune system is capable of surveillance and detection of cancer cells and then attack and destroy them on a regular basis under normal conditions. However, cancer cells seem to develop the ability of evading detection by the immune system and escape its response that ordinarily prevents the development of malignant tumors. There are several mechanisms by which the tumor cells can evade the effects of the immune system including the selection of tumor variants resistant to immune effectors (known as immune editing) and progressive formation of an immune suppressive environment within the tumor.

The tyrosine-protein phosphatase nonreceptor type (PTPN) 1 and 2 (also known as PTP1B and TC-PTP, respectively) are two closely related members of the class I nonreceptor protein tyrosine phosphatase family. Previously, it has been shown that both PTPN1 and PTPN2 are ubiquitously expressed with relatively high levels in immune cells. PTPN1 and PTPN2 are involved in regulation of signaling triggered by certain growth factor and cytokine receptors, such as epidermal growth factor receptor (EGFR), platelet-derived growth factor receptor (PDGFR), and insulin receptor (IR). Despite their similarity, the studies with PTPN1- and PTPN2-deficient mice suggest that their functions are not redundant. Ptpn1−/− mice are more sensitive to insulin and leptin and resistant to diet-induced obesity. However, Ptpn2−/− mice die within 3 to 5 weeks after birth as a result of hematopoietic defects and the development of progressive systemic inflammatory diseases. Furthermore, PTPN1/2 double-deficiency is lethal during embryonic development.

Protein tyrosine phosphatase non-receptor type 2 (PTPN2), also known as T cell protein tyrosine phosphatase (TC-PTP), is an intracellular member of the class 1 subfamily of phospho-tyrosine specific phosphatases that control multiple cellular regulatory processes by removing phosphate groups from tyrosine substrates. PTPN2 is ubiquitously expressed, but expression is highest in hematopoietic and placental cells. In humans, PTPN2 expression is controlled post-transcriptionally by the existence of two splice variants: a 45 kDa form that contains a nuclear localization signal at the C-terminus upstream of the splice junction, and a 48 kDa canonical form which has a C-terminal ER retention motif. The 45 kDa isoform can passively transfuse into the cytosol under certain cellular stress conditions. Both isoforms share an N-terminal phospho-tyrosine phosphatase catalytic domain. PTPN2 negatively regulates signaling of non-receptor tyrosine kinases (e.g. JAK1, JAK3), receptor tyrosine kinases (e.g. INSR, EGFR, CSF1R, PDGFR), transcription factors (e.g. STAT1, STAT3, STAT5a/b), and Src family kinases (e.g. Fyn, Lck). As a critical negative regulator of the JAK-STAT pathway, PTPN2 functions to directly regulate signaling through cytokine receptors, including IFNγ.

These findings suggest that enhancing IFNγ sensing and signaling through the inhibition of PTPN2 is a potential therapeutic strategy to improve the efficacy of cancer immunotherapy regimens. Unlike traditional cancer treatments (chemotherapy and radiation) that attack both cancer and healthy cells, immunotherapy can specifically target cancer cells; therefore, it promises fewer adverse effects. However, immunotherapy is still limited to the treatment of only few cancers and does not work for all patients. There are several promising approaches to treat cancer with immunotherapy.

The PTPN2 catalytic domain shares 74% sequence homology and similar enzymatic kinetics with another family member, the protein tyrosine phosphatase nonreceptor type 1 (PTPN1). Studies have determined a key role for PTPN1 in a primary mechanism for down-regulating both insulin and leptin receptor signaling pathways. Animal studies have determined that deficiency in PTPN1 has improved glucose regulation and lipid profiles. Animals deficient in PTPN1 are also resistant to weight gain even under a high fat diet. Thus, PTPN1 inhibitors are potentially useful for the treatment of type 2 diabetes, obesity, and metabolic syndrome.

There is a need for therapeutic agents that can inhibit PTPN1 and PTPN2. This invention is intended to fill this unmet need associated with current protein tyrosine phosphatase enzyme inhibition therapy.

### SUMMARY

A first aspect of the invention relates to compounds of Formula (I):

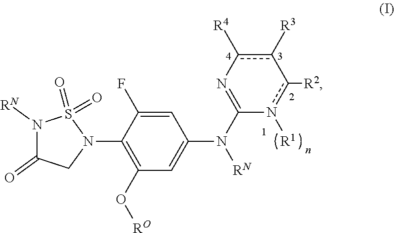

or a pharmaceutically acceptable salt, stereoisomer, solvate, prodrug, or tautomer thereof, wherein:
each bond [symbol] is independently selected from a single or a double bond;
n is 0 or 1;
provided that when bond 1 [symbol] 2 is a single bond, n is 1 and when bond 1 [symbol] 2 is a double bond, n is 0;
R¹ is selected from hydrogen, C₁-C₆ alkyl, C₂-C₆ alkenyl, C₂-C₆ alkynyl, C₁-C₆ alkyl-C(O)—, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
R² is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
R³ is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
or R² and R³ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵;
R⁴ is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
or R³ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵;
each R^{N} is independently selected from hydrogen, C₁-C₆ alkyl, —C(O)C₁-C₆ alkyl, —C(O)OC₁-C₆ alkyl;
R^{O} is selected from hydrogen, C₁-C₆ alkyl, —C(O)C₁-C₆ alkyl, —C(O)OC₁-C₆ alkyl, —CH₂-aryl;
each R⁵ is independently selected from halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁸;
R⁶ and R⁷ are each independently selected from hydrogen, C₁-C₆ alkyl, C₂-C₆ alkenyl, C₂-C₆ alkynyl, C₁-C₆ haloalkyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R⁸;
or R⁶ and R⁷ together with the atoms to which they are attached and any intervening atoms, form a 3-14 membered heterocycle, or a 5-6 membered heteroaryl, wherein the heterocycle or heteroaryl is optionally substituted with one or more R⁸;
each R⁸ is independently selected from halogen, OH, CN, NR⁶R⁷, ═NH, NO₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl;
wherein,
cycloalkyl is mono or polycyclic saturated carbon rings containing 3-18 carbon atoms;
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with PTPN1 and/or PTPN2. The method comprises administering to a patient in need of a treatment for diseases or disorders associated with PTPN1 and/or PTPN2 an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of PTPN1 and/or PTPN2. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting of PTPN1 and/or PTPN2.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder associated with PTPN1 and/or PTPN2.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with PTPN1 and/or PTPN2, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of PTPN1 and/or PTPN2 that are therapeutic agents in the treatment of diseases and disorders.

The present invention further provides compounds and compositions with an improved efficacy and safety profile relative to known inhibitors of PTPN1 and/or PTPN2.

The present invention further provides methods of treating a disease or disorder associated with PTPN1 and/or PTPN2, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of protein tyrosine phosphatase enzymes that are therapeutic agents in the treatment of diseases such as cancer and metabolic diseases.

The present invention provides inhibitors of PTPN1 and/or PTPN2 that are therapeutic agents in the treatment of diseases and disorders.

The present invention further provides compounds and compositions with an improved efficacy and safety profile relative to known protein tyrosine phosphatase enzyme inhibitors. The present disclosure also provides agents with novel mechanisms of action toward protein tyrosine phosphatase enzymes in the treatment of various types of diseases, including cancer and metabolic diseases.

The present invention further provides methods of preventing, treating, or ameliorating a disease, disorder, or condition selected from cancer, rheumatic disease, inflammatory disease, immune disease, metabolic disease or disorder, infectious disease, neurodegenerative disease, genetic disorder, cardiological disease.

The present invention further provides methods of treating a disease, disorder, or condition selected from Cancer; Oligoarticular Juvenile Idiopathic Arthritis; Rheumatoid Factor-Negative Polyarticular Juvenile Idiopathic Arthritis; Inflammatory Bowel Disease 20 (IBD20); Crohn's Disease; Immunodeficiency 31c (IMD31C); T-Cell Acute Lymphoblastic Leukemia; Inflammatory Bowel Disease; Inflammatory Bowel Disease 1 (IBD1); Celiac Disease 1 (CELIAC1); Body Mass Index Quantitative Trait Locus 11 (BMIQ11); Diabetes Mellitus; Type 2 Diabetes Mellitus (T2D); Rasopathy; Ovarian Cancer (OC); Bubonic Plague; Primary Mediastinal B-Cell Lymphoma; Leptin Deficiency or Dysfunction; Alzheimer Disease; Overnutrition; Noonan Syndrome; Noonan Syndrome With Multiple Lentigines; Rasopathy; Hypertension, Essential; Pancreatic Adenocarcinoma comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedures I, II, III or IV).

In some aspects, the present disclosure provides an intermediate as described herein, being suitable for use in a method for preparing a compound as described herein (e.g., the intermediate is selected from the intermediates described in Preparative part—P1-P49).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### DETAILED DESCRIPTION

The present disclosure provides methods of treating, preventing, or ameliorating a disease or disorder in which associated with PTPN1/PTPN2 by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bonded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —NH₂, —NO₂, —COOH, —CH₂CN, —O—(C₁-C₆) alkyl, (C₁-C₆) alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OP(O)(OH)₂, —OC(O)(C₁-C₆) alkyl, —C(O)(C₁-C₆) alkyl, —OC(O)O(C₁-C₆) alkyl, —NH((C₁-C₆) alkyl), —N((C₁-C₆) alkyl)₂, —NHC(O)(C₁-C₆) alkyl, —C(O)NH(C₁-C₆) alkyl, —S(O)₂(C₁-C₆) alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆)alkenyl, —O—(C₂-C₆) alkynyl, (C₂-C₆)alkenyl, (C₂-C₆)alkynyl, —OH, —OP(O)(OH)₂, —OC(O)(C₁-C₆)alkyl, —C(O)(C₁-C₆) alkyl, —OC(O)O(C₁-C₆)alkyl, —NH₂, —NH((C₁-C₆)alkyl), —N((C₁-C₆)alkyl)₂, —S(O)₂—(C₁-C₆) alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo [1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo [1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7(4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “0” in the chain, i.e., —O(alkyl). Examples of alkoxy groups include without limitation, methoxy, ethoxy, propoxy, butoxy, tert-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-1H-indenyl, spiro[3.5]nonyl, spiro [5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “halogenalkyl” as used herein refers to an alkyl group, as defined herein, which is substituted one or more halogen. Examples of halogenalkyl groups include, but are not limited to, trifluoromethyl, difluoromethyl, pentafluoroethyl, trichloromethyl, etc.

The term “halogenalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C≡N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diastereomers.

The present disclosure also contemplates isotopically-labelled compounds of Formula (I) (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula (I) can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents, and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound.

The term “salt’ refers to pharmaceutically acceptable salts.

The term “pharmaceutically acceptable salt” also refers to a salt of the compositions of the present disclosure having an acidic functional group, such as a carboxylic acid functional group, and a base.

“Inhibitors of PTPN1 and/or PTPN2” as used herein refer to compounds of Formula (I) and/or compositions comprising a compound of Formula (I) which inhibit of PTPN1 and/or PTPN2.

As used herein, the term “cancer” refers to all types of cancer, neoplasm or malignant tumors found in mammals, including leukemia, lymphoma, carcinomas, and sarcomas. Exemplary cancers that may be treated with a compound, pharmaceutical composition, or method provided herein include lymphoma, B-cell lymphoma, heavy chain disease, alpha chain disease, gamma chain disease, mu chain disease, Waldenstrom's macroglobulinemia, benign monoclonal gammopathy, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, acoustic neuroma, retinoblastoma, astrocytoma, craniopharyngioma, hemangioblastoma, pinealoma, ependymoma, oligodendroglioma, meningioma, glioma, or melanoma. Additional examples include, cancer of the thyroid, endocrine system, brain, breast, cervix, colon, head & neck, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, immunocytic amyloidosis, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The amount of compound of composition described herein needed for achieving a therapeutic effect may be determined empirically in accordance with conventional procedures for the particular purpose. Generally, for administering therapeutic agents (e.g. compounds or compositions of Formula (I) (and/or additional agents) described herein) for therapeutic purposes, the therapeutic agents are given at a pharmacologically effective dose.

A “pharmacologically effective amount”, “pharmacologically effective dose”, “therapeutically effective amount”, or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In one aspect, the present disclosure provides compounds of Formula (I) and salts, stereoisomers, solvates, prodrugs, isotopic derivatives, and tautomers thereof:

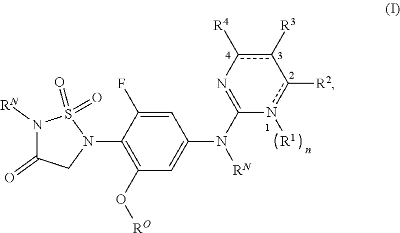

Wherein R¹, R², R³, R⁴, R^{N}, R^{O}, and n as described herein.

It is understood that, for a compound of Formula (I), R¹, R², R³, R⁴, R^{N}, R^{O}, and n can each be, where applicable, selected from the groups described herein, and any group described herein for any of R¹, R², R³, R⁴, R^{N}, R^{O}, and n can be combined, where applicable, with any group described herein for one or more of the remainders of R¹, R², R³, R⁴, R^{N}, R^{O}, and n.

In some embodiments,
each bond [symbol] is independently selected from a single or a double bond;
n is 0 or 1;
provided that when bond 1 [symbol] 2 is a single bond, n is 1 and when bond 1 [symbol] 2 is a double bond, n is 0;
R¹ is selected from hydrogen, C₁-C₆ alkyl, C₂-C₆ alkenyl, C₂-C₆ alkynyl, C₁-C₆ alkyl-C(O)—, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
R² is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
R³ is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
or R² and R³ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵;
R⁴ is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
or R³ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵;
each R^{N} is independently selected from hydrogen, C₁-C₆ alkyl, —C(O)C₁-C₆ alkyl, —C(O)OC₁-C₆ alkyl;
R^{O} is selected from hydrogen, C₁-C₆ alkyl, —C(O)C₁-C₆ alkyl, —C(O)OC₁-C₆ alkyl, —CH₂-aryl;
each R⁵ is independently selected from halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁸;
R⁶ and R⁷ are each independently selected from hydrogen, C₁-C₆ alkyl, C₂-C₆ alkenyl, C₂-C₆ alkynyl, C₁-C₆ haloalkyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R⁸;
or R⁶ and R⁷ together with the atoms to which they are attached and any intervening atoms, form a 3-14 membered heterocycle, or a 5-6 membered heteroaryl, wherein the heterocycle or heteroaryl is optionally substituted with one or more R⁸
each R⁸ is independently selected from halogen, OH, CN, NR⁶R⁷, ═NH, NO₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl.

In some embodiments, each bond [symbol] is independently selected from a single or a double bond.

In some embodiments, bond 1 [symbol] 2 and bond 3 [symbol] 4 are single bonds.

In some embodiments, bond 1 [symbol] 2 and bond 3 [symbol] 4 are double bonds.

In some embodiments, bond 1 [symbol] 2 is a single bond and bond 3 [symbol] 4 is a double bond.

In some embodiments, bond 1 [symbol] 2 is a double bond and bond 3 [symbol] 4 is a single bond.

In some embodiments, bond 1 [symbol] 2 is a single bond and bond 3 [symbol] 4 is a single bond.

In some embodiments, bond 1 [symbol] 2 is a double bond and bond 3 [symbol] 4 is a double bond.

In some embodiments, n is an integer selected from 0 and 1.

In some embodiments, n is 0.

In some embodiments, n is 1.

In some embodiments, when bond 1 [symbol] 2 is a double bond n is 0.

In some embodiments, when bond 1 [symbol] 2 is a single bond n is 1.

It would be understood by a person of ordinary skill in the art that if bond 1 [symbol] 2 is a double bond and bond 3 [symbol] 4 is a double bond the ring comprising these bonds is aromatic system and each bond in pyrimidine ring is an aromatic bond:

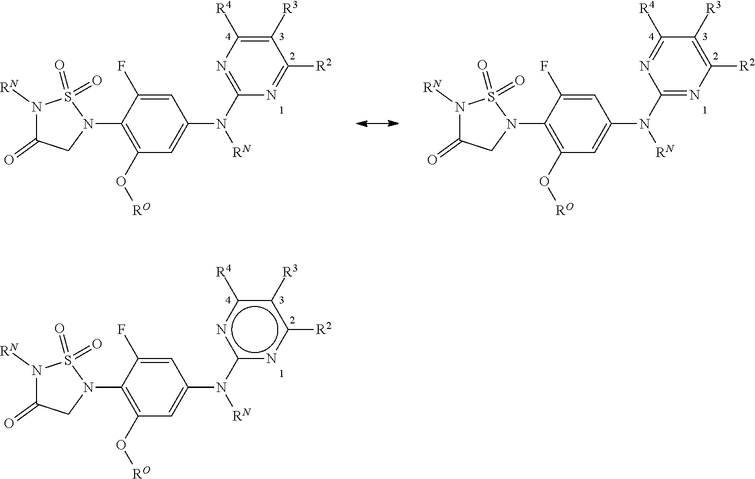

In some embodiments, R¹ is selected from hydrogen, C₁-C₆ alkyl, C₂-C₆ alkenyl, C₂-C₆ alkynyl, C₁-C₆ alkyl-C(O)—, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵.

In some embodiments, R¹ is hydrogen.

In some embodiments, R² is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵.

In some embodiments, R² is H.

In some embodiments, R² is —OH.

In some embodiments, R² is C₁-C₆ alkyl.

In some embodiments, R² is methyl. In some embodiments, R² is ethyl. In some embodiments, R² is propyl. In some embodiments, R² is n-propyl. In some embodiments, R² is iso-propyl. In some embodiments, R² is butyl. In some embodiments, R² is n-butyl. In some embodiments, R² is iso-butyl. In some embodiments, R² is n-butyl. In some embodiments, R² is sec-butyl. In some embodiments, R² is tert-butyl. In some embodiments, R² is pentyl. In some embodiments, R² is n-pentyl. In some embodiments, R² is n-hexyl.

In some embodiments, R² is CH₃.

In some embodiments, R² is C₁-C₆ alkoxy.

In some embodiments, R² is methoxy. In some embodiments, R² is ethoxy. In some embodiments, R² is propoxy. In some embodiments, R² is —OCH₂CH₂CH₃. In some embodiments, R² is —OCH(CH₃)₂. In some embodiments, R² is butoxy. In some embodiments, R² is —OCH₂CH₂CH₂CH₃. In some embodiments, R² is —OCH₂CH(CH₃)₂. In some embodiments, R² is —OC(CH₃)₃. In some embodiments, R² is pentoxy. In some embodiments, R² is hexoxy.

In some embodiments, R² is —OCH₃.

In some embodiments, R² is —OC₂H₅.

In some embodiments, R² is —O—CH₂CH₂CH₃.

In some embodiments, R² is

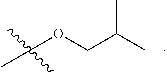 [SMILES: *OCC(C)C]

In some embodiments, R² is

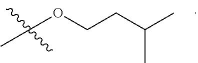 [SMILES: *OCCC(C)C]

In some embodiments, R² is —NR⁶R⁷.

In some embodiments, R² is —NH₂.

In some embodiments, R² is —NHCH₃.

In some embodiments, R² is —N(CH₃)₂.

In some embodiments, R² is

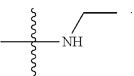 [SMILES: *NCC]

In some embodiments, R² is

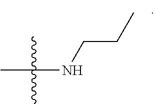 [SMILES: *NCCC]

In some embodiments, R² is

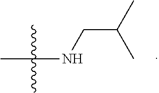 [SMILES: *NCC(C)C]

In some embodiments, R² is

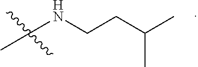 [SMILES: *NCCC(C)C]

In some embodiments, R² is —N(CH₃)₂.

In some embodiments, R² is

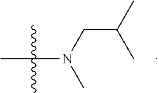 [SMILES: *N(C)CC(C)C]

In some embodiments, R² is

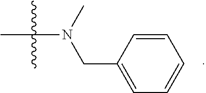 [SMILES: *N(C)Cc1ccccc1]

In some embodiments, R² is heterocyclyl.

In some embodiments, R² is

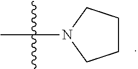 [SMILES: *N1CCCC1]

In some embodiments, R² is

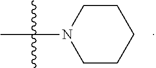 [SMILES: *N1CCCCC1]

In some embodiments, R² is C₁-C₆ alkoxy substituted with one group R⁵.

In some embodiments, R² is C₁-C₆ alkoxy substituted with one cycloalkyl.

In some embodiments, R² is

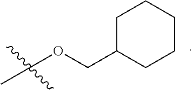 [SMILES: *OCC1CCCCC1]

In some embodiments, R³ is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵.

In some embodiments, R³ is H.

In some embodiments, R³ is halogen.

In some embodiments, R³ is —F.

In some embodiments, R³ is —Cl.

In some embodiments, R³ is —Br.

In some embodiments, R³ is —I.

In some embodiments, R³ is —C₁-C₆ alkyl.

In some embodiments, R³ is methyl. In some embodiments, R³ is ethyl. In some embodiments, R³ is propyl. In some embodiments, R³ is n-propyl. In some embodiments, R³ is iso-propyl. In some embodiments, R³ is butyl. In some embodiments, R³ is n-butyl. In some embodiments, R³ is iso-butyl. In some embodiments, R³ is n-butyl. In some embodiments, R³ is sec-butyl. In some embodiments, R³ is tert-butyl. In some embodiments, R³ is pentyl. In some embodiments, R³ is n-pentyl. In some embodiments, R³ is n-hexyl.

In some embodiments, R³ is —CH₃.

In some embodiments, R³ is —CH₂CH₃.

In some embodiments, R³ is —CH₂CH₂CH₃.

In some embodiments, R³ is

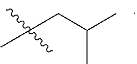 [SMILES: *CC(C)C]

In some embodiments, R³ is

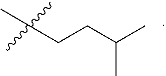 [SMILES: *CCC(C)C]

In some embodiments, R³ is cycloalkyl.

In some embodiments, R³ is

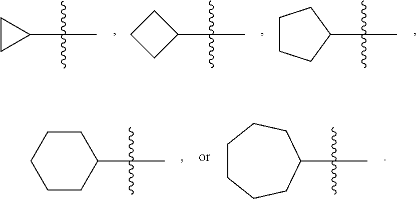

In some embodiments, R³ is

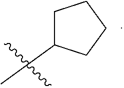 [SMILES: *C1CCCC1]

In some embodiments, R² and R³ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵.

In some embodiments, R² and R³ together with the atoms to which they are attached and any intervening atoms, form a 6-10 membered aryl, wherein the aryl is optionally substituted with one or more R⁵.

In some embodiments, R² and R³ together with the atoms to which they are attached and any intervening atoms, form

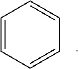

In some embodiments, R² and R³ together with the atoms to which they are attached and any intervening atoms, form a 3-10 membered cycloalkyl, wherein the cycloalkyl is optionally substituted with one or more R⁵.

In some embodiments, R² and R³ together with the atoms to which they are attached and any intervening atoms, form

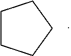

In some embodiments, R² and R³ together with the atoms to which they are attached and any intervening atoms, form

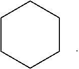

In some embodiments, R is selected from hydrogen, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵.

In some embodiments, R⁴ is H.

In some embodiments, R⁴ is —OH.

In some embodiments, R⁴ is C₁-C₆ alkyl.

In some embodiments, R⁴ is methyl. In some embodiments, R⁴ is ethyl. In some embodiments, R⁴ is propyl. In some embodiments, R⁴ is n-propyl. In some embodiments, R⁴ is iso-propyl. In some embodiments, R⁴ is butyl. In some embodiments, R⁴ is n-butyl. In some embodiments, R⁴ is iso-butyl. In some embodiments, R⁴ is n-butyl. In some embodiments, R⁴ is sec-butyl. In some embodiments, R⁴ is tert-butyl. In some embodiments, R⁴ is pentyl. In some embodiments, R⁴ is n-pentyl. In some embodiments, R⁴ is n-hexyl.

In some embodiments, R⁴ is CH₃.

In some embodiments, R⁴ is C₁-C₆ alkoxy.

In some embodiments, R⁴ is methoxy. In some embodiments, R⁴ is ethoxy. In some embodiments, R⁴ is propoxy. In some embodiments, R⁴ is —OCH₂CH₂CH₃. In some embodiments, R⁴ is —OCH(CH₃)₂. In some embodiments, R⁴ is butoxy. In some embodiments, R⁴ is —OCH₂CH₂CH₂CH₃. In some embodiments, R⁴ is —OCH₂CH(CH₃)₂. In some embodiments, R⁴ is —OC(CH₃)₃. In some embodiments, R⁴ is pentoxy. In some embodiments, R⁴ is hexoxy.

In some embodiments, R⁴ is —OCH₃.

In some embodiments, R⁴ is —OC₂H₅.

In some embodiments, R⁴ is —O—CH₂CH₂CH₃.

In some embodiments, R⁴ is

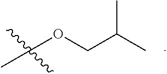 [SMILES: *OCC(C)C]

In some embodiments, R⁴ is

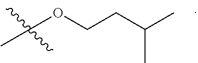 [SMILES: *OCCC(C)C]

In some embodiments, R⁴ is —NR⁶R⁷.

In some embodiments, R⁴ is —NH₂.

In some embodiments, R⁴ is —NHCH₃.

In some embodiments, R⁴ is —N(CH₃)₂.

In some embodiments, R⁴ is

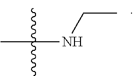 [SMILES: *NCC]

In some embodiments, R⁴ is

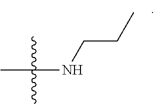 [SMILES: *NCCC]

In some embodiments, R⁴ is

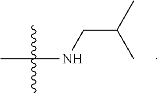 [SMILES: *NCC(C)C]

In some embodiments, R⁴ is

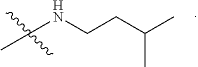 [SMILES: *NCCC(C)C]

In some embodiments, R⁴ is —N(CH₃)₂.

In some embodiments, R⁴ is

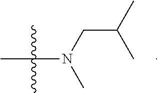 [SMILES: *N(C)CC(C)C]

In some embodiments, R⁴ is

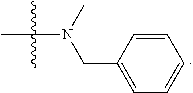 [SMILES: *N(C)Cc1ccccc1]

In some embodiments, R⁴ is heterocyclyl.

In some embodiments, R⁴ is

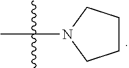 [SMILES: *N1CCCC1]

In some embodiments, R⁴ is

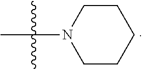 [SMILES: *N1CCCCC1]

In some embodiments, R⁴ is C₁-C₆ alkoxy substituted with one group R⁵.

In some embodiments, R⁴ is C₁-C₆ alkoxy substituted with one cycloalkyl.

In some embodiments, R⁴ is

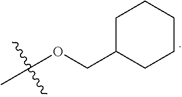 [SMILES: *OCC1CCCCC1]

In some embodiments, R³ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵.

In some embodiments, R³ and R⁴ together with the atoms to which they are attached and any intervening atoms, form a 6-10 membered aryl, wherein the aryl is optionally substituted with one or more R⁵.

In some embodiments, R³ and R⁴ together with the atoms to which they are attached and any intervening atoms, form

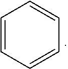

In some embodiments, R³ and R⁴ together with the atoms to which they are attached and any intervening atoms, form a 3-10 membered cycloalkyl, wherein the cycloalkyl is optionally substituted with one or more R⁵.

In some embodiments, R³ and R⁴ together with the atoms to which they are attached and any intervening atoms, form

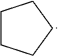

In some embodiments, R³ and R⁴ together with the atoms to which they are attached and any intervening atoms, form

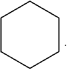

In some embodiments, each R^{N} is independently selected from hydrogen, C₁-C₆ alkyl, —C(O)C₁-C₆ alkyl, and —C(O)OC₁-C₆ alkyl.

In some embodiments, one R^{N} is H.

In some embodiments, each R^{N} is H.

In some embodiments, R^{O} is selected from hydrogen, C₁-C₆ alkyl, —C(O)C₁-C₆ alkyl, —C(O)OC₁-C₆ alkyl, —CH₂-aryl.

In some embodiments, R^{O} is H.

In some embodiments, each R⁵ is independently selected from halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁸.

In some embodiments, R⁵ is halogen.

In some embodiments, R⁵ is F. In some embodiments, R⁵ is Cl. In some embodiments, R⁵ is Br. In some embodiments, R⁵ is I.

In some embodiments, R⁵ is —OH.

In some embodiments, R⁵ is —CN.

In some embodiments, R⁵ is —NO₂.

In some embodiments, R⁵ is —NR⁶R⁷.

In some embodiments, R⁵ is —NH₂, —N(H)CH₃, —N(CH₃)₂, —N(H)CH₂CH₃, or —N(H)CH₂CH₂CH₃.

In some embodiments, R⁵ is C₁-C₆ alkyl optimally substituted optionally substituted with one or more R⁸.

In some embodiments, R⁵ is C₁-C₆ alkyl.

In some embodiments, R⁵ is methyl. In some embodiments, R⁵ is ethyl. In some embodiments, R⁵ is propyl. In some embodiments, R⁵ is n-propyl. In some embodiments, R⁵ is i-propyl. In some embodiments, R⁵ is butyl. In some embodiments, R⁵ is n-butyl. In some embodiments, R⁵ is iso-butyl. In some embodiments, R⁵ is n-butyl. In some embodiments, R⁵ is sec-butyl. In some embodiments, R⁵ is tert-butyl. In some embodiments, R⁵ is pentyl. In some embodiments, R⁵ is n-pentyl. In some embodiments, R⁵ is n-hexyl.

In some embodiments, R⁵ is C₂-C₆ alkenyl optionally substituted with one or more R⁸.

In some embodiments, R⁵ is C₂-C₆ alkynyl optionally substituted with one or more R⁸.

In some embodiments, R⁵ is C₁-C₆ alkoxy optionally substituted with one or more R⁸.

In some embodiments, R⁵ is C₁-C₆ alkoxy.

In some embodiments, R⁵ is methoxy. In some embodiments, R⁵ is ethoxy. In some embodiments, R⁵ is propoxy. In some embodiments, R⁵ is —OCH₂CH₂CH₃. In some embodiments, R⁵ is —OCH(CH₃)₂. In some embodiments, R⁵ is butoxy. In some embodiments, R⁵ is —OCH₂CH₂CH₂CH₃. In some embodiments, R⁵ is —OCH₂CH(CH₃)₂. In some embodiments, R⁵ is —OC(CH₃)₃. In some embodiments, R⁵ is pentoxy. In some embodiments, R⁵ is hexoxy.

In some embodiments, R⁵ is —C(O)OR⁶. In some embodiments, R⁵ is —C(O)OCH₃. In some embodiments, R⁵ is —C(O)OCH₂CH₃. In some embodiments, R⁵ is —C(O)OCH(CH₃)₂. In some embodiments, R⁵ is —C(O)OC(CH₃)₃.

In some embodiments, R⁵ is —C(O)NR⁶R⁷. In some embodiments, R⁵ is —C(O)NH₂. In some embodiments, R⁵ is —C(O)N(H)CH₃. In some embodiments, R⁵ is —C(O)N(CH₃)₂.

In some embodiments, R⁵ is

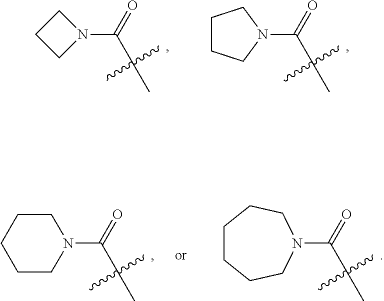 [SMILES: *C(=O)N1CCC1 ; *C(=O)N1CCCC1 ; *C(=O)N1CCCCC1 ; *C(=O)N1CCCCCC1]

In some embodiments, R⁵ is cycloalkyl optimally substituted with one or more R⁸.

In some embodiments, R⁵ is C₃-C₁₈ cycloalkyl.

In some embodiments, R⁵ is C₃-C₁₀ cycloalkyl.

In some embodiments, R⁵ is

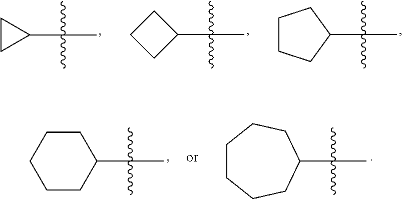

In some embodiments, R⁵ is —O-cycloalkyl.

In some embodiments, R⁵ is

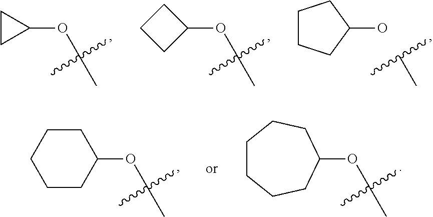

In some embodiments, R⁵ is aryl optionally substituted with one or more R⁸.

In some embodiments, R⁵ is

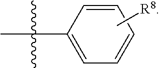

In some embodiments, R⁵ is

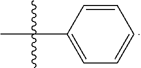 [SMILES: *c1ccccc1]

In some embodiments, R⁵ is heterocyclyl optionally substituted with one or more R⁸.

In some embodiments, R⁵ is heterocyclyl.

In some embodiments, R⁵ is selected from

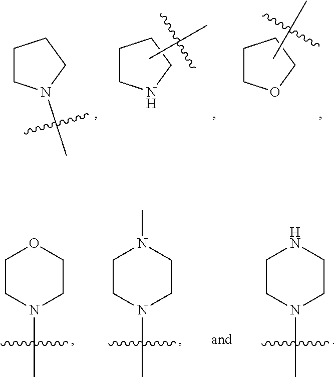

In some embodiments, R⁵ is heteroaryl optionally substituted with one or more R⁸.

In some embodiments, R⁵ is heteroaryl.

In some embodiments, R⁵ is selected from

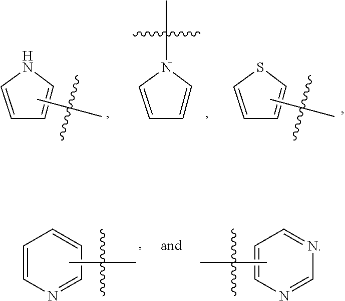

In some embodiments, R⁶ is selected from hydrogen, C₁-C₆ alkyl, C₂-C₆ alkenyl, C₂-C₆ alkynyl, C₁-C₆ haloalkyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R⁸.

In some embodiments, R⁷ is selected from hydrogen, C₁-C₆ alkyl, C₂-C₆ alkenyl, C₂-C₆ alkynyl, C₁-C₆ haloalkyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R⁸.

In some embodiments, R⁶ and R⁷ together with the atoms to which they are attached and any intervening atoms, form a 3-14 membered heterocycle, or a 5-6 membered heteroaryl, wherein the heterocycle or heteroaryl is optionally substituted with one or more R⁸.

In some embodiments, R⁸ is independently selected from halogen, OH, CN, NR⁶R⁷, ═NH, NO₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl.

In some embodiments, R⁸ is halogen.

In some embodiments, R⁸ is F.

In some embodiments, R⁸ is Cl.

In some embodiments, R⁸ is Br.

In some embodiments, R⁸ is I.

In some embodiments, R⁸ is —OH.

In some embodiments, R⁸ is —CN.

In some embodiments, R⁸ is —NR⁶R⁷.

In some embodiments, R⁸ is ═NH.

In some embodiments, R⁸ is NO₂.

In some embodiments, R⁸ is C₁-C₆ alkyl.

In some embodiments, R⁸ is methyl. In some embodiments, R⁸ is ethyl. In some embodiments, R⁸ is propyl. In some embodiments, R⁸ is n-propyl. In some embodiments, R⁸ is i-propyl. In some embodiments, R⁸ is butyl. In some embodiments, R⁸ is n-butyl. In some embodiments, R⁸ is iso-butyl. In some embodiments, R⁸ is sec-butyl. In some embodiments, R⁸ is tert-butyl. In some embodiments, R⁸ is pentyl. In some embodiments, R⁸ is n-pentyl. In some embodiments, R⁸ is hexyl. In some embodiments, R⁸ is n-hexyl.

In some embodiments, R⁸ is C₁-C₆ alkoxy.

In some embodiments, R⁸ is methoxy. In some embodiments, R⁸ is ethoxy. In some embodiments, R⁸ is propoxy. In some embodiments, R⁸ is —OCH₂CH₂CH₃. In some embodiments, R⁸ is —OCH(CH₃)₂. In some embodiments, R⁸ is butoxy. In some embodiments, R⁸ is —OCH₂CH₂CH₂CH₃. In some embodiments, R⁸ is —OCH₂CH(CH₃)₂. In some embodiments, R⁸ is —OC(CH₃)₃. In some embodiments, R⁸ is pentoxy. In some embodiments, R⁸ is hexoxy.

In some embodiments, R⁸ is C₁-C₆ alkyl-C₁-C₆ alkoxy.

In some embodiments, R⁸ is —CH₂—O—CH₃. In some embodiments, R⁸ is —CH₂—O—CH₂CH₃. In some embodiments, R⁸ is —CH₂CH₂—O—CH₃. In some embodiments, R⁸ is —CH₂CH₂—O—CH₂CH₃.

In some embodiments, R⁸ is C₁-C₆ alkyl-NHC₁-C₆ alkyl.

In some embodiments, R⁸ is —CH₂—NH—CH₃. In some embodiments, R⁸ is —CH₂—NH—CH₂CH₃. In some embodiments, R⁸ is —CH₂CH₂—NH—CH₃. In some embodiments, R⁸ is —CH₂CH₂—NH—CH₂CH₃.

In some embodiments, R⁸ is cycloalkyl.

In some embodiments, R⁸ is

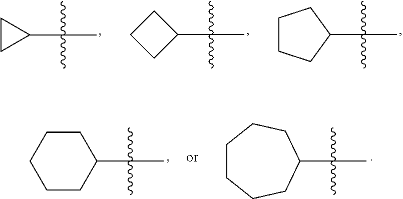

In some embodiments, R⁸ is heterocyclyl.

In some embodiments, R⁸ is selected from

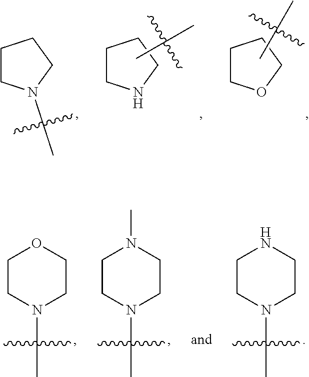

In some embodiments, R⁸ is aryl.

In some embodiments, R⁸ is

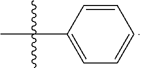 [SMILES: *c1ccccc1]

In some embodiments, R⁸ is heteroaryl.

In some embodiments, R⁸ is selected from

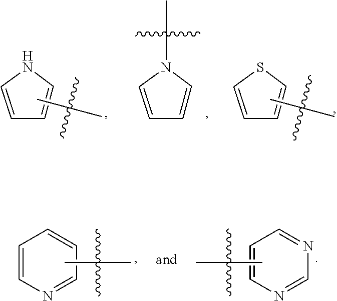

In some embodiments, the compound is of Formula (I-I):

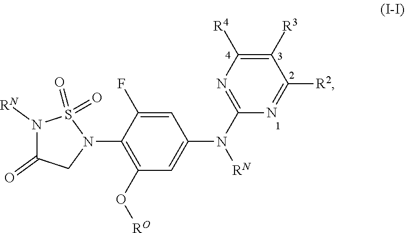

or a pharmaceutically acceptable salt, stereoisomer, solvate, prodrug, isotopic derivative, or tautomer thereof wherein all variables are as defined herein.

In some embodiments, the compound is of Formula (I-II):

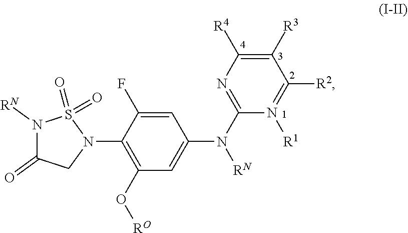

or a pharmaceutically acceptable salt, stereoisomer, solvate, prodrug, isotopic derivative, or tautomer thereof wherein all variables are as defined herein.

In some embodiments, the compound is of Formula (I-I-H):

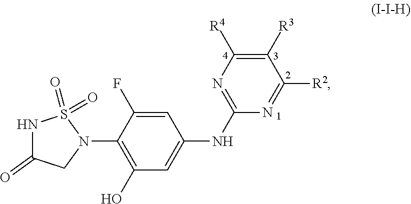

or a pharmaceutically acceptable salt, stereoisomer, solvate, prodrug, isotopic derivative, or tautomer thereof wherein all variables are as defined herein.

In some embodiments, the compound is of Formula (I-II-H):

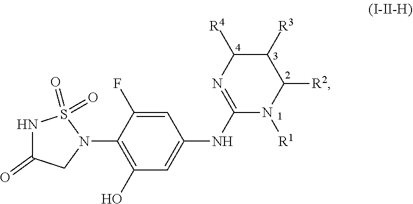

or a pharmaceutically acceptable salt, stereoisomer, solvate, prodrug, isotopic derivative, or tautomer thereof wherein all variables are as defined herein.

In some embodiments, the compound is of Formula (I-I-A):

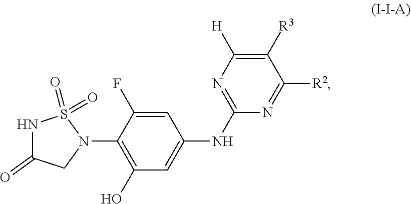

or a pharmaceutically acceptable salt, stereoisomer, solvate, prodrug, isotopic derivative, or tautomer thereof wherein all variables are as defined herein.

In some embodiments, the compound is of Formula (I-I-A-1):

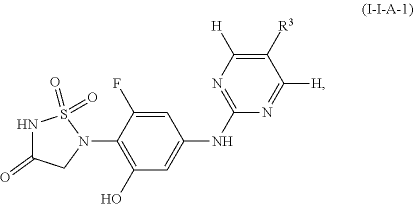

or a pharmaceutically acceptable salt, stereoisomer, solvate, prodrug, isotopic derivative, or tautomer thereof wherein R³ is as defined herein.

In some embodiments, the compound is of Formula (I-I-A-2):

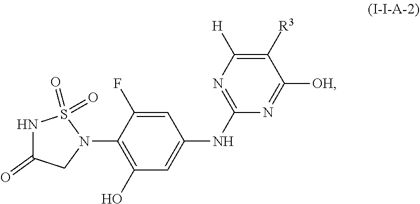

or a pharmaceutically acceptable salt, stereoisomer, solvate, prodrug, isotopic derivative, or tautomer thereof wherein R³ is as defined herein.

In some embodiments, the compound is of Formula (I-I-A-3):

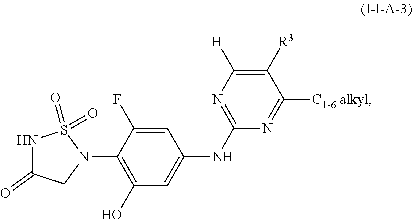

or a pharmaceutically acceptable salt, stereoisomer, solvate, prodrug, isotopic derivative, or tautomer thereof wherein R³ is as defined herein.

In some embodiments, the compound is of Formula (I-I-A-3-1):

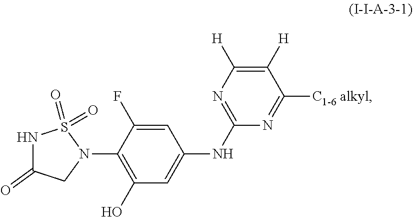

or a pharmaceutically acceptable salt, stereoisomer, solvate, prodrug, isotopic derivative, or tautomer thereof.

In some embodiments, the compound is of Formula (I-I-A-4):

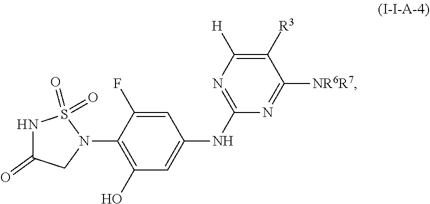

or a pharmaceutically acceptable salt, stereoisomer, solvate, prodrug, isotopic derivative, or tautomer thereof wherein all variables are as defined herein.

In some embodiments, the compound is of Formula (I-I-A-4-1):

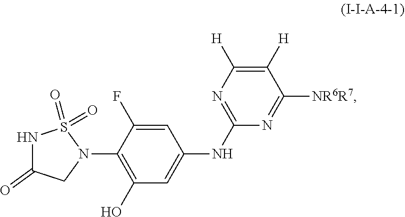

or a pharmaceutically acceptable salt, stereoisomer, solvate, prodrug, isotopic derivative, or tautomer thereof wherein all variables are as defined herein.

In some embodiments, the compound is of Formula (I-I-A-4-1-1):

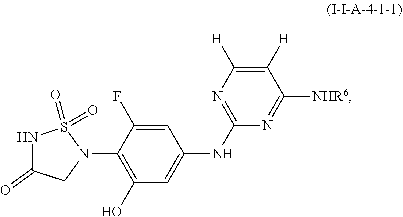

or a pharmaceutically acceptable salt, stereoisomer, solvate, prodrug, isotopic derivative, or tautomer thereof wherein R⁶ is as defined herein.

In some embodiments, the compound is of Formula (I-I-B):

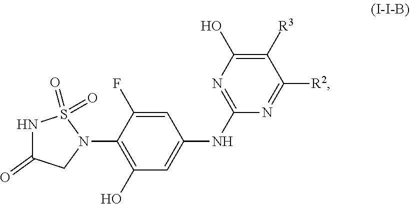

or a pharmaceutically acceptable salt, stereoisomer, solvate, pro rug, isotopic derivative, or tautomer thereof wherein all variables are as defined herein.

In some embodiments, the compound is of Formula (Q-I-A-2):

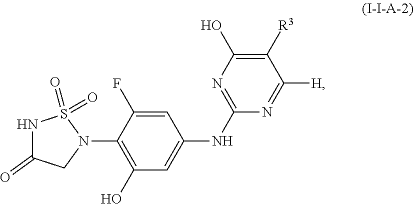

or a pharmaceutically acceptable salt, stereoisomer, solvate, prodrug, isotopic derivative, or tautomer thereof wherein R³ is as defined herein.

In some embodiments, the compound is of Formula (I-I-B-1):

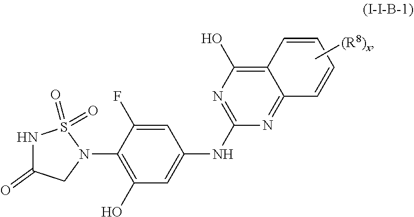

or a pharmaceutically acceptable salt, stereoisomer, solvate, prodrug, isotopic derivative, or tautomer thereof wherein x is an integer selected from 0, 1, 2, 3, and 4 and R⁸ is as defined herein.

In some embodiments, the compound is of Formula (I-I-B-2):

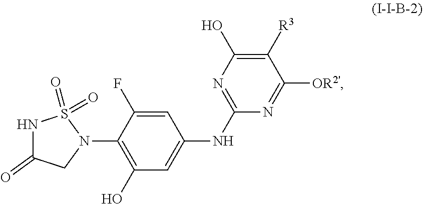

or a pharmaceutically acceptable salt, stereoisomer, solvate, prodrug, isotopic derivative, or tautomer thereof, wherein R^{2′} is selected from H, C₁-C₆ alkyl, —C₁-C₆ alkyl-C₃-C₁₀ cycloalkyl and R³ is as defined herein.

In some embodiments, the compound is of Formula (I-I-B-2-1):

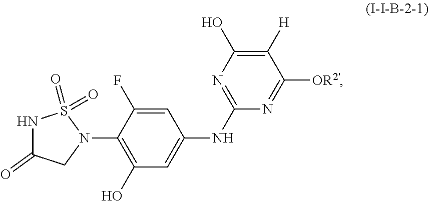

or a pharmaceutically acceptable salt, stereoisomer, solvate, prodrug, isotopic derivative, or tautomer thereof, wherein R^{2′} is selected from H, C₁-C₆ alkyl, —C₁-C₆ alkyl-C₃-C₁₀ cycloalkyl.

In some embodiments, the compound is of Formula (I-I-B-2-2):

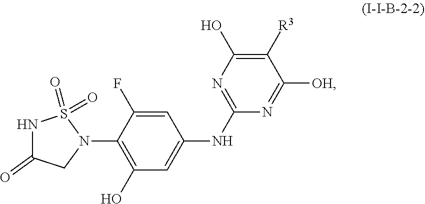

or a pharmaceutically acceptable salt, stereoisomer, solvate, prodrug, isotopic derivative, or tautomer thereof wherein R³ is as defined herein.

In some embodiments, the compound is of Formula (I-I-B-3):

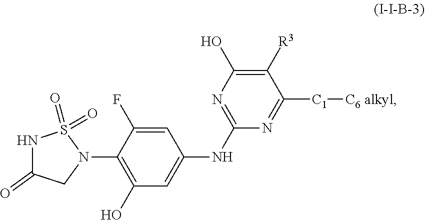

or a pharmaceutically acceptable salt, stereoisomer, solvate, prodrug, isotopic derivative, or tautomer thereof wherein R³ is as defined herein.

In some embodiments, the compound is of Formula (I-I-B-3-1):

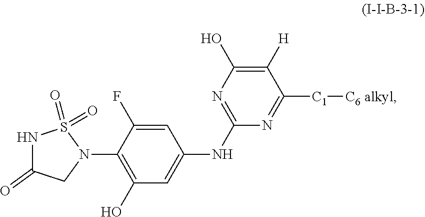

or a pharmaceutically acceptable salt, stereoisomer, solvate, prodrug, isotopic derivative, or tautomer thereof.

In some embodiments, the compound is of Formula (I-I-B-3-2):

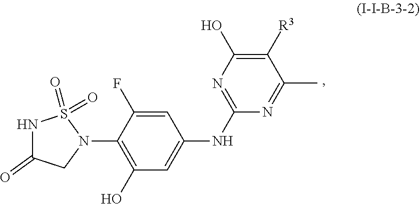

or a pharmaceutically acceptable salt, stereoisomer, solvate, prodrug, isotopic derivative, or tautomer thereof wherein R³ is as defined herein.

In some embodiments, the compound is of Formula (I-I-B-4):

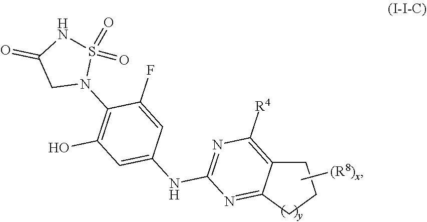

or a pharmaceutically acceptable salt, stereoisomer, solvate, prodrug, isotopic derivative, or tautomer thereof wherein y is an integer selected from 0, 1, 2, 3, 4; x is an integer selected from 0, 1, 2, 3, 4, 5, 6; and R⁸ is as defined herein.

In some embodiments, the compound is of Formula (I-I-B-4′):

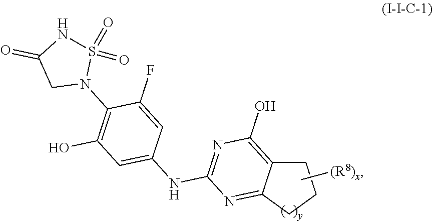

or a pharmaceutically acceptable salt, stereoisomer, solvate, prodrug, isotopic derivative, or tautomer thereof wherein y is an integer selected from 0, 1, 2, 3, 4; x is an integer selected from 0, 1, 2, 3, 4, 5, 6; R⁴ and R⁸ are as defined herein.

In some embodiments, the compound is of Formula (I-I-D):

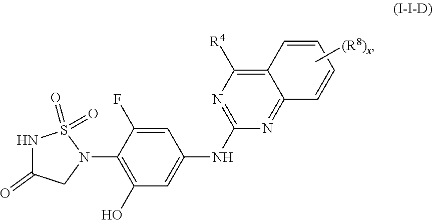

or a pharmaceutically acceptable salt, stereoisomer, solvate, prodrug, isotopic derivative, or tautomer thereof wherein x is an integer selected from 0, 1, 2, 3, and 4 and R⁴ and R⁸ are as defined herein.

In some embodiments, the compound is of Formula (I-II-A):

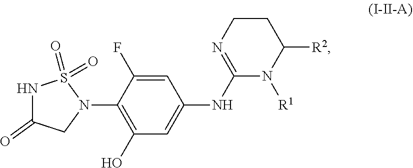

or a pharmaceutically acceptable salt, stereoisomer, solvate, prodrug, isotopic derivative, or tautomer thereof, wherein all variables are as defined herein.

In some embodiments, the compound is of Formula (I-II-A′):

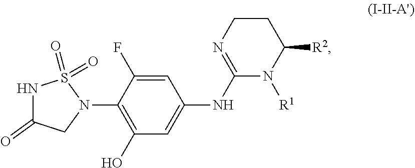

or a pharmaceutically acceptable salt, stereoisomer, solvate, prodrug, isotopic derivative, or tautomer thereof, wherein all variables are as defined herein.

In some embodiments, the compound is of Formula (I-II-A″):

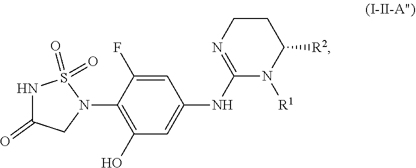

or a pharmaceutically acceptable salt, stereoisomer, solvate, prodrug, isotopic derivative, or tautomer thereof, wherein all variables are as defined herein.

In some embodiments, the compound is of Formula (II-A-H):

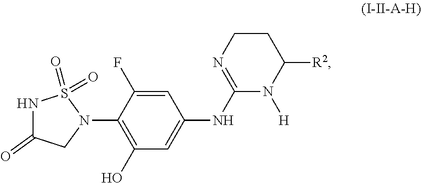

or a pharmaceutically acceptable salt, stereoisomer, solvate, prodrug, isotopic derivative, or tautomer thereof, wherein R² is as defined herein.

In some embodiments, the compound is of Formula (I-II-A-H′):

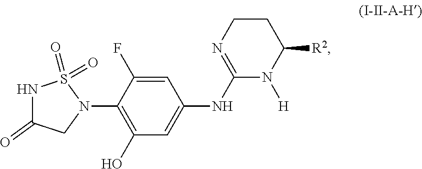

or a pharmaceutically acceptable salt, stereoisomer, solvate, prodrug, isotopic derivative, or tautomer thereof, wherein R² is as defined herein.

In some erphodiments, the compound is of Formula (I-IL-A-H″)

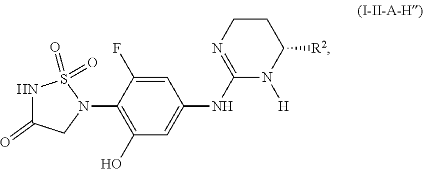

or a pharmaceutically acceptable salt, stereoisomer, solvate, prodrug, isotopic derivative, or tautomer thereof, wherein R² is as defined herein.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl)amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

In some embodiments, the compound is selected from the compounds described in Table 1 and pharmaceutically acceptable salts, stereoisomers, solvates, prodrugs, isotopic derivatives, or tautomers thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of the compounds described in Table 1 and pharmaceutically acceptable salts thereof.

In some embodiments, the compound is selected from the compounds described in Table 1.

TABLE 1
Certain examples of the compound of Formula (I)

| # | IUPAC Name |
|---|---|
| 1 | 5-[2-fluoro-6-hydroxy-4-(pyrimidin-2-ylamino)phenyl]-1,1-dioxo-1,2,5- |
| | thiadiazolidin-3-one |
| 2 | 5-[2-fluoro-6-hydroxy-4-(1,4,5,6-tetrahydropyrimidin-2-ylamino)phenyl]-1,1- |
| | dioxo-1,2,5-thiadiazolidin-3-one |
| 3 | 5-[2-fluoro-6-hydroxy-4-[(6-methyl-1,4,5,6-tetrahydropyrimidin-2- |
| | yl)amino]phenyl]-1,1-dioxo-1,2,5-thiadiazolidin-3-one |
| 4 | 5-[2-fluoro-6-hydroxy-4-[(4-methylpyrimidin-2-yl)amino]phenyl]-1,1-dioxo- |
| | 1,2,5-thiadiazolidin-3-one |
| 5 | 5-[4-[(5-chloropyrimidin-2-yl)amino]-2-fluoro-6-hydroxy-phenyl]-1,1-dioxo- |
| | 1,2,5-thiadiazolidin-3-one |
| 6 | 5-[2-fluoro-6-hydroxy-4-[(4-hydroxy-5-methyl-pyrimidin-2-yl)amino]phenyl]- |
| | 1,1-dioxo-1,2,5-thiadiazolidin-3-one |
| 7 | 5-[2-fluoro-6-hydroxy-4-[(4-hydroxypyrimidin-2-yl)amino]phenyl]-1,1-dioxo- |
| | 1,2,5-thiadiazolidin-3-one |
| 8 | 5-[2-fluoro-6-hydroxy-4-[(4-hydroxyquinazolin-2-yl)amino]phenyl]-1,1-dioxo- |
| | 1,2,5-thiadiazolidin-3-one |
| 9 | 5-[2-fluoro-6-hydroxy-4-[(4-hydroxy-6-methoxy-pyrimidin-2- |
| | yl)amino]phenyl]-1,1-dioxo-1,2,5-thiadiazolidin-3-one |
| 10 | 5-[2-fluoro-6-hydroxy-4-[(4-hydroxy-6-methyl-pyrimidin-2-yl)amino]phenyl]- |
| | 1,1-dioxo-1,2,5-thiadiazolidin-3-one |
| 11 | 5-[4-[[4-[benzyl(methyl)amino]pyrimidin-2-yl]amino]-2-fluoro-6-hydroxy- |
| | phenyl]-1,1-dioxo-1,2,5-thiadiazolidin-3-one |
| 12 | 5-[2-fluoro-6-hydroxy-4-[(4-hydroxy-5-isobutyl-pyrimidin-2-yl)amino]phenyl]- |
| | 1,1-dioxo-1,2,5-thiadiazolidin-3-one |
| 13 | 5-[4-[(4,6-dihydroxy-5-isobutyl-pyrimidin-2-yl)amino]-2-fluoro-6-hydroxy- |
| | phenyl]-1,1-dioxo-1,2,5-thiadiazolidin-3-one |
| 14 | 5-[2-fluoro-6-hydroxy-4-[[4-(isopentylamino)pyrimidin-2-yl]amino]phenyl]- |
| | 1,1-dioxo-1,2,5-thiadiazolidin-3-one |
| 15 | 5-[2-fluoro-6-hydroxy-4-[[4-(methylamino)pyrimidin-2-yl]amino]phenyl]-1,1- |
| | dioxo-1,2,5-thiadiazolidin-3-one |
| 16 | 5-[2-fluoro-6-hydroxy-4-[(4-hydroxy-6-isopentyloxy-pyrimidin-2- |
| | yl)amino]phenyl]-1,1-dioxo-1,2,5-thiadiazolidin-3-one |
| 17 | 5-[2-fluoro-6-hydroxy-4-[[4-hydroxy-6-(isopentylamino)pyrimidin-2- |
| | yl]amino]phenyl]-1,1-dioxo-1,2,5-thiadiazolidin-3-one |
| 18 | 5-[2-fluoro-6-hydroxy-4-[(4-hydroxy-5-isopentyl-pyrimidin-2- |
| | yl)amino]phenyl]-1,1-dioxo-1,2,5-thiadiazolidin-3-one |
| 19 | 5-[2-fluoro-6-hydroxy-4-[(4-hydroxy-6-pyrrolidin-1-yl-pyrimidin-2- |
| | yl)amino]phenyl]-1,1-dioxo-1,2,5-thiadiazolidin-3-one |
| 20 | 5-[2-fluoro-6-hydroxy-4-[(4-hydroxy-5-isobutyl-6-methyl-pyrimidin-2- |
| | yl)amino]phenyl]-1,1-dioxo-1,2,5-thiadiazolidin-3-one |
| 21 | 5-[2-fluoro-6-hydroxy-4-[(4-hydroxy-6-methyl-5-propyl-pyrimidin-2- |
| | yl)amino]phenyl]-1,1-dioxo-1,2,5-thiadiazolidin-3-one |
| 22 | 5-[4-[(4,6-dihydroxy-5-propyl-pyrimidin-2-yl)amino]-2-fluoro-6-hydroxy- |
| | phenyl]-1,1-dioxo-1,2,5-thiadiazolidin-3-one |
| 23 | 5-[4-[(5-ethyl-4,6-dihydroxy-pyrimidin-2-yl)amino]-2-fluoro-6-hydroxy- |
| | phenyl]-1,1-dioxo-1,2,5-thiadiazolidin-3-one |
| 24 | 5-[4-[(5-cyclopentyl-4,6-dihydroxy-pyrimidin-2-yl)amino]-2-fluoro-6-hydroxy- |
| | phenyl]-1,1-dioxo-1,2,5-thiadiazolidin-3-one |
| 25 | 5-[2-fluoro-6-hydroxy-4-[[4-(propylamino)pyrimidin-2-yl]amino]phenyl]-1,1- |
| | dioxo-1,2,5-thiadiazolidin-3-one |
| 26 | 5-[4-[[4-(ethylamino)pyrimidin-2-yl]amino]-2-fluoro-6-hydroxy-phenyl]-1,1- |
| | dioxo-1,2,5-thiadiazolidin-3-one |
| 27 | 5-[2-fluoro-6-hydroxy-4-[[4-(isobutylamino)pyrimidin-2-yl]amino]phenyl]-1,1- |
| | dioxo-1,2,5-thiadiazolidin-3-one |
| 28 | 5-[2-fluoro-6-hydroxy-4-[[4-hydroxy-6-(isobutylamino)pyrimidin-2- |
| | yl]amino]phenyl]-1,1-dioxo-1,2,5-thiadiazolidin-3-one |
| 29 | 5-[2-fluoro-6-hydroxy-4-[[4-hydroxy-6-(propylamino)pyrimidin-2- |
| | yl]amino]phenyl]-1,1-dioxo-1,2,5-thiadiazolidin-3-one |
| 30 | 5-[4-[[4-(dimethylamino)-6-hydroxy-pyrimidin-2-yl]amino]-2-fluoro-6- |
| | hydroxy-phenyl]-1,1-dioxo-1,2,5-thiadiazolidin-3-one |
| 31 | 5-[2-fluoro-6-hydroxy-4-[[4-hydroxy-6-[isobutyl(methyl)amino]pyrimidin-2- |
| | yl]amino]phenyl]-1,1-dioxo-1,2,5-thiadiazolidin-3-one |
| 32 | 5-[2-fluoro-6-hydroxy-4-[[4-hydroxy-6-(1-piperidyl)pyrimidin-2- |
| | yl]amino]phenyl]-1,1-dioxo-1,2,5-thiadiazolidin-3-one |
| 33 | 5-[4-[[4-(cyclohexylmethoxy)-6-hydroxy-pyrimidin-2-yl]amino]-2-fluoro-6- |
| | hydroxy-phenyl]-1,1-dioxo-1,2,5-thiadiazolidin-3-one |
| 34 | 5-[4-[(4-ethoxy-6-hydroxy-pyrimidin-2-yl)amino]-2-fluoro-6-hydroxy-phenyl]- |
| | 1,1-dioxo-1,2,5-thiadiazolidin-3-one |
| 35 | 5-[2-fluoro-6-hydroxy-4-[(4-hydroxy-6-propoxy-pyrimidin-2-yl)amino]phenyl]- |
| | 1,1-dioxo-1,2,5-thiadiazolidin-3-one |
| 36 | 5-[2-fluoro-6-hydroxy-4-[(4-hydroxy-6-isobutoxy-pyrimidin-2- |
| | yl)amino]phenyl]-1,1-dioxo-1,2,5-thiadiazolidin-3-one |
| 37 | 5-[4-[(5-ethyl-4-hydroxy-6-methyl-pyrimidin-2-yl)amino]-2-fluoro-6-hydroxy- |
| | phenyl]-1,1-dioxo-1,2,5-thiadiazolidin-3-one |
| 38 | 5-[2-fluoro-6-hydroxy-4-[(4-hydroxy-5,6-dimethyl-pyrimidin-2- |
| | yl)amino]phenyl]-1,1-dioxo-1,2,5-thiadiazolidin-3-one |
| 39 | 5-[2-fluoro-6-hydroxy-4-[(4-hydroxy-5,6,7,8-tetrahydroquinazolin-2- |
| | yl)amino]phenyl]-1,1-dioxo-1,2,5-thiadiazolidin-3-one |
| 40 | 5-[2-fluoro-6-hydroxy-4-[(4-hydroxy-6,7-dihydro-5H-cyclopenta[d]pyrimidin- |
| | 2-yl)amino]phenyl]-1,1-dioxo-1,2,5-thiadiazolidin-3-one |
| 41 | 5-[2-fluoro-6-hydroxy-4-[(4-hydroxy-5-isopentyl-6-methyl-pyrimidin-2- |
| | yl)amino]phenyl]-1,1-dioxo-1,2,5-thiadiazolidin-3-one |
| 42 | 5-[4-[(4-chloro-6-hydroxy-5-isobutyl-pyrimidin-2-yl)amino]-2-fluoro-6- |
| | hydroxy-phenyl]-1,1-dioxo-1,2,5-thiadiazolidin-3-one |

In some embodiments, the compound is a pharmaceutically acceptable salt of any one of the compounds described in Table 1.

In some embodiments, the compound is a lithium salt, sodium salt, potassium salt, calcium salt, or magnesium salt of any one of the compounds described in Table 1.

In some embodiments, the compound is a sodium salt or potassium salt of any one of the compounds described in Table 1.

In some embodiments, the compound is a salt of any acid described in the Table 2 and any one of the compounds described in Table 1.

TABLE 2
Pharmaceutical acceptable acid forming salts with the Compound of Formula (I).

| | | |
|---|---|---|
| 1-hydroxy-2-naphthoic acid | ethanesulfonic acid | methanesulfonic acid |
| 2,2-dichloroacetic acid | formic acid | naphthalene-1,5-disulfonic acid |
| 2-hydroxyethanesulfonic acid | fumaric acid | naphthalene-2-sulfonic acid |
| 2-oxoglutaric acid | galactaric acid | nicotinic acid |
| 4-acetamidobenzoic acid | gentisic acid | nitric acid |
| 4-aminosalicylic acid | glucoheptonic acid (D) | oleic acid |
| acetic acid | gluconic acid (D) | oxalic acid |
| adipic acid | glucuronic acid (D) | palmitic acid |
| ascorbic acid (L) | glutamic acid | pamoic acid |
| aspartic acid (L) | glutaric acid | phosphoric acid |
| benzenesulfonic acid | glycerophosphoric acid | proprionic acid |
| benzoic acid | glycolic acid | pyroglutamic acid (−L) |
| camphoric acid (+) | hippuric acid | salicylic acid |
| camphor-10-sulfonic acid (+) | hydrobromic acid | sebacic acid |
| capric acid (decanoic acid) | hydrochloric acid | stearic acid |
| caproic acid (hexanoic acid) | isobutyric acid | succinic acid |
| caprylic acid (octanoic acid) | lactic acid (DL) | sulfuric acid |
| carbonic acid | lactobionic acid | tartaric acid (+L) |
| cinnamic acid | lauric acid | thiocyanic acid |
| citric acid | maleic acid | toluenesulfonic acid (p) |
| cyclamic acid | malic acid (−L) | undecylenic acid |
| dodecylsulfuric acid | malonic acid | |
| ethane-1,2-disulfonic acid | mandelic acid (DL) | |

In some embodiments, the compound is a salt of acetic acid and any one of the compounds described in Table 1.

In some embodiments, the compound is a salt of adipic acid and any one of the compounds described in Table 1.

In some embodiments, the compound is a salt of ascorbic acid (L) and any one of the compounds described in Table 1.

In some embodiments, the compound is a salt of hydrobromic acid and any one of the compounds described in Table 1.

In some embodiments, the compound is a salt of hydrochloric acid and any one of the compounds described in Table 1.

In some embodiments, the compound is a salt of citric acid and any one of the compounds described in Table 1.

In some embodiments, the compound is a salt of glutamic acid and any one of the compounds described in Table 1.

In some embodiments, the compound is a salt of oxalic acid and any one of the compounds described in Table 1.

In some embodiments, the compound is a salt of formic acid and any one of the compounds described in Table 1.

In some embodiments, the compound is a salt of sulfuric acid and any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H ¹³C, ¹⁴C, ¹⁵N, ¹⁸O, ²⁹SI, ³¹P, and ³⁴S. In some embodiments, the isotopic derivative is a deuterium labeled compound (i.e., being enriched with ²H with regard to one or more atoms thereof).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵I, ¹²⁹I, ¹³¹I, ¹³⁵I, ³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³S, ³S and/or ³⁶S labeled reagent for anon-isotope labeled reagent.

A compound of the disclosure or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³S, ³⁴S, ³⁵S, and ³⁶S atom(s) is within the scope of the disclosure. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1100 Daltons. More usually, the molecular weight of the compound will be less than 1000, for example less than 900, or less than 800, or less than 700, or less than 600, or less than 500.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemi-hydrate, a mono-hydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning Calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compound of Formula (I) include all such forms. For the avoidance of doubt, where a compound can exist in one of several tautomeric forms, and only one is specifically described or shown, all others are nevertheless embraced by Formula (I). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

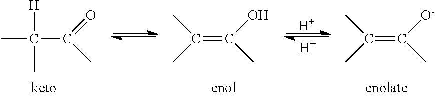

Compounds of any one of the Formulae disclosed herein containing an amine function may also form N-oxides. A reference herein to a compound of Formula (I) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically-produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl)piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures (General schemes I, II, III, IV) which comprise different sequences of assembling intermediates or compounds. Starting materials are either commercially available or made by known procedures in the reported literature or as illustrated below.

### General Procedure

In general, the compound of the Formula (I) can be prepared using sequences of reaction well known to those skilled in the art of organic synthesis. General scheme I showed possible synthetic sequences for the preparation of compound of the Formula (I) presented below:

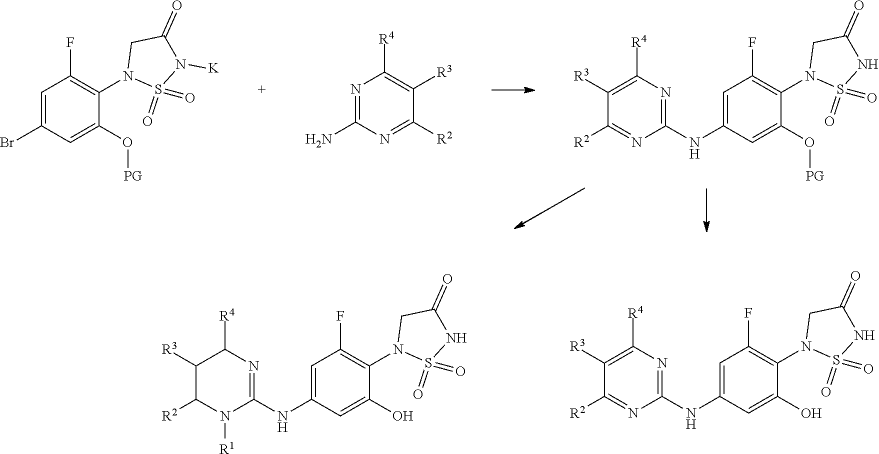

All reagents may be commercially available compounds itself or products of synthesis from commercially available reagents. For each compound in preparation may be used one step or multistep synthetic procedures, including but not limited procedures described herein in preparative part.

Another useful General scheme II, showed possible synthetic sequences for the preparation of compound of the Formula (I) presented below:

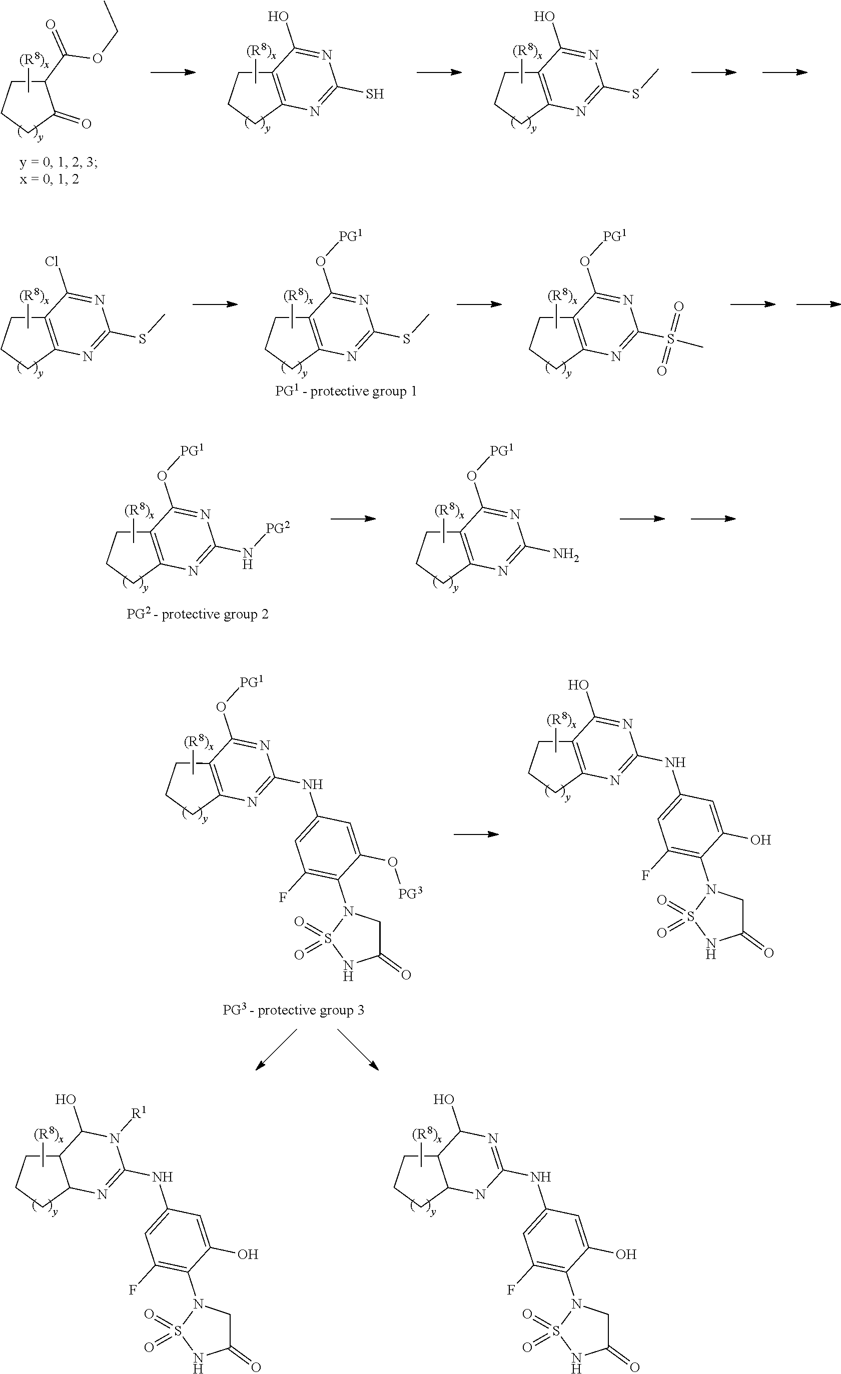

Another useful General scheme III, showed possible synthetic sequences for the preparation of compound of the Formula (I) presented below:

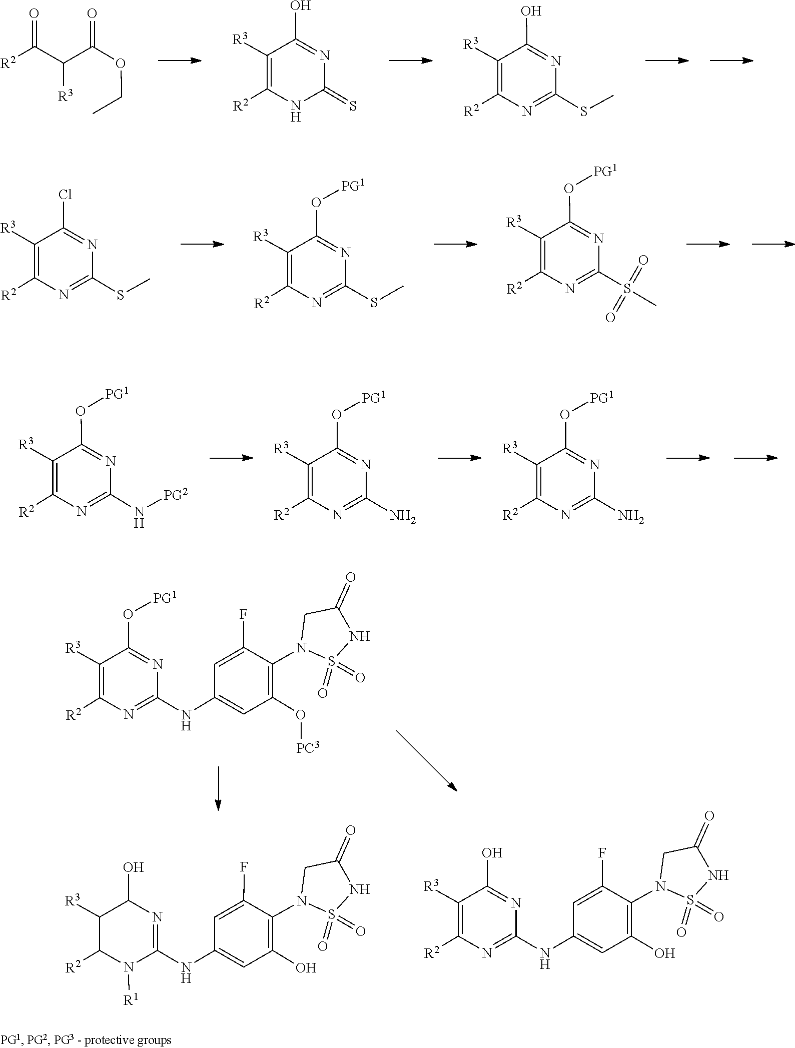

Another useful General scheme IV, showed possible synthetic sequences for the preparation of compound of the Formula (I) presented below:

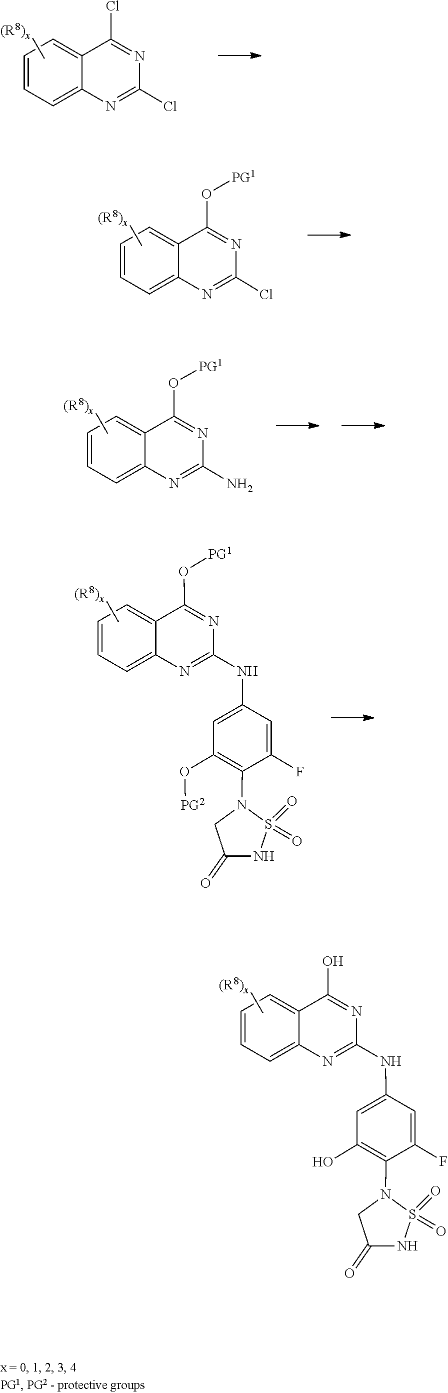

It should be obvious for specialist in this field that any of compound of Formula (I) obtained according to the procedures described above may be a subject for further transformation and modification that will be led to other compound of Formula (I).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from the Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio)propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols—such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base—depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and ε-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a PTPN1/PTPN2 related condition referred to herein, slow its progression and/or reduce the symptoms associated with the condition.

A therapeutically effective amount of a compound of the present disclosure for use in therapy is an amount sufficient to treat a PTPN1/PTPN2 related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or subject and the route of administration, according to well-known principles of medicine.

### Methods of Use

In some aspects, the present disclosure provides a method of inhibition of PTPN1/PTPN2 (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with PTPN1/PTPN2. In some embodiments, the disease or disorder is a disease or disorder in which PTPN1/PTPN2 is implicated.

The compounds of the invention are inhibitors of PTPN1/PTPN2.

In some embodiments, the compounds, compositions, and methods disclosed herein are used in the prevention or treatment of a disease, disorder, or condition. Exemplary diseases, disorders, or conditions include, but are not limited to cancer, type-2 diabetes, metabolic syndrome, obesity, or a metabolic disease.

The compounds of the invention are also useful in treating diseases associated with the PTPN1/PTPN2. For example, diseases and conditions treatable according to the methods of the invention include Cancer; Oligoarticular Juvenile Idiopathic Arthritis; Rheumatoid Factor-Negative Polyarticular Juvenile Idiopathic Arthritis; Inflammatory Bowel Disease 20 (IBD20); Crohn's Disease; Immunodeficiency 31c (IMD31C); T-Cell Acute Lymphoblastic Leukemia; Inflammatory Bowel Disease; Inflammatory Bowel Disease 1 (IBD1); Celiac Disease 1 (CELIAC1); Body Mass Index Quantitative Trait Locus 11 (BMIQ11); Diabetes Mellitus; Type 2 Diabetes Mellitus (T2D); Rasopathy; Ovarian Cancer (OC); Bubonic Plague; Primary Mediastinal B-Cell Lymphoma; Leptin Deficiency or Dysfunction; Alzheimer Disease; Overnutrition; Noonan Syndrome; Noonan Syndrome With Multiple Lentigines; Rasopathy; Hypertension, Essential.

In some embodiments, the disease or disorder is Cancer.

As used herein, “cancer” refers to human cancers and carcinomas, sarcomas, adenocarcinomas (e.g., papillary adenocarcinomas), lymphomas, leukemias, melanomas, etc., including solid and lymphoid cancers, kidney, breast, lung, bladder, colon, ovarian, prostate, pancreas, stomach, brain, head and neck, skin, uterine, testicular, glioma, esophagus, liver cancer, including hepatocarcinoma, lymphoma, including Bacute lymphoblastic lymphoma, non-Hodgkin's lymphomas (e.g., Burkitt's, Small Cell, and Large Cell lymphomas), Hodgkin's lymphoma, leukemia (including AML, ALL, and CML), and/or multiple myeloma. In some further instances, “cancer” refers to lung cancer, breast cancer, ovarian cancer, epithelial ovarian cancer, leukemia, lymphoma, melanoma, pancreatic cancer, sarcoma, bladder cancer, bone cancer, biliary tract cancer, adrenal gland cancer, salivary gland cancer, bronchus cancer, oral cancer, cancer of the oral cavity or pharynx, laryngeal cancer, renal cancer, gynecologic cancers, brain cancer, central nervous system cancer, peripheral nervous system cancer, cancer of the hematological tissues, small bowel or appendix cancer, cervical cancer, colon cancer, esophageal cancer, gastric cancer, liver cancer, head and neck cancer, kidney cancer, myeloma, thyroid cancer, prostate cancer, metastatic cancer, or carcinoma.

As used herein, the term “cancer” refers to all types of cancer, neoplasm or malignant tumors found in mammals, including leukemia, lymphoma, carcinomas and sarcomas. Exemplary cancers that may be treated with a compound, pharmaceutical composition, or method provided herein include lymphoma, B-cell lymphoma, heavy chain disease, alpha chain disease, gamma chain disease, mu chain disease, Waldenstrom's macroglobulinemia, benign monoclonal gammopathy, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g., ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g., non-small cell lung carcinoma, squamous cell lung carcinoma, adenocarcinoma, large cell lung carcinoma, small cell lung carcinoma, carcinoid, sarcoma), glioblastoma multiforme, acoustic neuroma, retinoblastoma, astrocytoma, craniopharyngioma, hemangioblastoma, pinealoma, ependymoma, oligodendroglioma, meningioma, glioma, or melanoma. Additional examples include, cancer of the thyroid, endocrine system, brain, breast, cervix, colon, head & neck, liver, kidney, lung, nonsmall cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, immunocytic amyloidosis, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

In some embodiments, the diseases or disorder is Ovarian Cancer (OC).

In some embodiments, the disease or disorder is Leukemia.

In some embodiments, the disease or disorder is T-Cell Acute Lymphoblastic Leukemia.

The term “leukemia” refers broadly to progressive, malignant diseases of the bloodforming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). Exemplary leukemias that may be treated with a compound, pharmaceutical composition, or method provided herein include, for example, chronic leukemia, acute nonlymphocytic leukemia, acute lymphocytic leukemia, 8-cell chronic byniphoeytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, acute myelocytic leukemia, chronic myelocytic leukemia, leukemia cutis, embryonal leukemia, eosinophilic leukemia, erythroleukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocyte leukemia, micromyeloblastic leukemia, monocytic leukemia, myeloblasts leukemia, myelocytic leukemia, myeloid granulocytic leukemia, myelomonocytic leukemia, Naegeli leukemia, plasma cell leukemia, multiple myeloma, plasmacytic leukemia, polycythemia vera, promyelocytic leukemia, Rieder cell leukemia, Schilling's leukemia, stem cell leukemia, subleukemic leukemia, or undifferentiated cell leukemia.

In some embodiments, the disease or disorder is Oligoarticular Juvenile Idiopathic Arthritis.

In some embodiments, the disease or disorder is Rheumatoid Factor-Negative Polyarticular Juvenile Idiopathic Arthritis.

In some embodiments, the diseases or disorder is Inflammatory Bowel Disease 20 (IBD20).

In some embodiments, the diseases or disorder is Crohn's Disease.

In some embodiments, the diseases or disorder is Immunodeficiency 31c (IMD31C).

In some embodiments, the diseases or disorder is Inflammatory Bowel Disease.

In some embodiments, the diseases or disorder is Inflammatory Bowel Disease 1 (IBD1).

In some embodiments, the diseases or disorder is Celiac Disease 1 (CELIAC1).

In some embodiments, the diseases or disorder is Body Mass Index Quantitative Trait Locus 11 (BMIQ11).

In some embodiments, the diseases or disorder is Diabetes Mellitus.

In some embodiments, the diseases or disorder is Type 2 Diabetes Mellitus (T2D).

In some embodiments, the diseases or disorder is Rasopathy.

In some aspects, the present disclosure provides a method of treating or preventing a Cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Leukemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a T-Cell Acute Lymphoblastic Leukemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Oligoarticular Juvenile Idiopathic Arthritis in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Rheumatoid Factor-Negative Polyarticular Juvenile Idiopathic Arthritis in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Inflammatory Bowel Disease 20 (IBD20) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Crohn's Disease in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Immunodeficiency 31c (IMD31C) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Celiac Disease 1 (CELIAC1) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Body Mass Index Quantitative Trait Locus 11 (BMIQ11) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Diabetes Mellitus in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Type 2 Diabetes Mellitus (T2D) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Rasopathy in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating or preventing a Ovarian Cancer (OC) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Leukemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a T-Cell Acute Lymphoblastic Leukemia in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Oligoarticular Juvenile Idiopathic Arthritis in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Rheumatoid Factor-Negative Polyarticular Juvenile Idiopathic Arthritis in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an Inflammatory Bowel Disease 20 (IBD20) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Crohn's Disease in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an Immunodeficiency 31c (IMD31C) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an Inflammatory Bowel Disease in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an Inflammatory Bowel Disease 1 (IBD1) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Celiac Disease 1 (CELIAC1) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Body Mass Index Quantitative Trait Locus 11 (BMIQ11) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Diabetes Mellitus in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Type 2 Diabetes Mellitus (T2D) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a Rasopathy in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating an Ovarian Cancer (OC) in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting of PTPN1/PTPN2 (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a Cancer in a subject in need thereof.

In some aspects, the present disclosure provides a compound of the present disclosure or a pharmaceutically acceptable salt thereof for use in treating or preventing a Leukemia in a subject in need thereof.

In some aspects, the present disclosure provides a compound of the present disclosure or a pharmaceutically acceptable salt thereof for use in treating or preventing a T-Cell Acute Lymphoblastic Leukemia in a subject in need thereof.

In some aspects, the present disclosure provides a compound of the present disclosure or a pharmaceutically acceptable salt thereof for use in treating or preventing an Oligoarticular Juvenile Idiopathic Arthritis in a subject in need thereof.

In some aspects, the present disclosure provides a compound of the present disclosure or a pharmaceutically acceptable salt thereof for use in treating or preventing an Inflammatory Bowel Disease 20 (IBD20) in a subject in need thereof.

In some aspects, the present disclosure provides a compound of the present disclosure or a pharmaceutically acceptable salt thereof for use in treating or preventing a Crohn's Disease in a subject in need thereof.

In some aspects, the present disclosure provides a compound of the present disclosure or a pharmaceutically acceptable salt thereof for use in treating an Immunodeficiency 31c (IMD3IC) in a subject in need thereof.

In some aspects, the present disclosure provides a compound of the present disclosure or a pharmaceutically acceptable salt thereof for use in treating an Inflammatory Bowel Disease in a subject in need thereof.

In some aspects, the present disclosure provides a compound of the present disclosure or a pharmaceutically acceptable salt thereof for use in treating an Inflammatory Bowel Disease 1 (IBD1) in a subject in need thereof.

In some aspects, the present disclosure provides a compound of the present disclosure or a pharmaceutically acceptable salt thereof for use in treating a Celiac Disease 1 (CELIAC1) in a subject in need thereof.

In some aspects, the present disclosure provides a compound of the present disclosure or a pharmaceutically acceptable salt thereof for use in treating a Body Mass Index Quantitative Trait Locus 11 (BMIQ11) in a subject in need thereof.

In some aspects, the present disclosure provides a compound of the present disclosure or a pharmaceutically acceptable salt thereof for use in treating a Diabetes Mellitus in a subject in need thereof.

In some aspects, the present disclosure provides a compound of the present disclosure or a pharmaceutically acceptable salt thereof for use in treating a Type 2 Diabetes Mellitus (T2D) in a subject in need thereof.

In some aspects, the present disclosure provides a compound of the present disclosure or a pharmaceutically acceptable salt thereof for use in treating a Rasopathy in a subject in need thereof.

In some aspects, the present disclosure provides a compound of the present disclosure or a pharmaceutically acceptable salt thereof for use in treating an Ovarian Cancer (OC) in a subject in need thereof.

In some aspects, the present disclosure provides use of a compound of the present disclosure or a pharmaceutically acceptable salt thereof in the manufacture of a medicament for inhibiting of PTPN1/PTPN2 (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a Leukemia in a subject in need thereof.

In some aspects, the present disclosure provides use of a compound of the present disclosure or a pharmaceutically acceptable salt thereof in the manufacture of a medicament for treating or preventing a T-Cell Acute Lymphoblastic Leukemia in a subject in need thereof.

In some aspects, the present disclosure provides use of a compound of the present disclosure or a pharmaceutically acceptable salt thereof in the manufacture of a medicament for treating or preventing a Cancer in a subject in need thereof.

In some aspects, the present disclosure provides use of a compound of the present disclosure or a pharmaceutically acceptable salt thereof in the manufacture of a medicament for treating or preventing an Oligoarticular Juvenile Idiopathic Arthritis in a subject in need thereof.

In some aspects, the present disclosure provides use of a compound of the present disclosure or a pharmaceutically acceptable salt thereof in the manufacture of a medicament for treating or preventing a Rheumatoid Factor-Negative Polyarticular Juvenile Idiopathic Arthritis in a subject in need thereof.

In some aspects, the present disclosure provides use of a compound of the present disclosure or a pharmaceutically acceptable salt thereof in the manufacture of a medicament for treating or preventing an Inflammatory Bowel Disease 20 (IBD20) in a subject in need thereof.

In some aspects, the present disclosure provides use of a compound of the present disclosure or a pharmaceutically acceptable salt thereof in the manufacture of a medicament for treating a Crohn's Disease in a subject in need thereof.

In some aspects, the present disclosure provides use of a compound of the present disclosure or a pharmaceutically acceptable salt thereof in the manufacture of a medicament for treating an Immunodeficiency 31c (IMD31C) in a subject in need thereof.

In some aspects, the present disclosure provides use of a compound of the present disclosure or a pharmaceutically acceptable salt thereof in the manufacture of a medicament for treating an Inflammatory Bowel Disease in a subject in need thereof.

In some aspects, the present disclosure provides use of a compound of the present disclosure or a pharmaceutically acceptable salt thereof in the manufacture of a medicament for treating an Inflammatory Bowel Disease 1 (IBD1) in a subject in need thereof.

In some aspects, the present disclosure provides use of a compound of the present disclosure or a pharmaceutically acceptable salt thereof in the manufacture of a medicament for treating a Celiac Disease 1 (CELIAC1) in a subject in need thereof.

In some aspects, the present disclosure provides use of a compound of the present disclosure or a pharmaceutically acceptable salt thereof in the manufacture of a medicament for treating a Body Mass Index Quantitative Trait Locus 11 (BMIQ11) in a subject in need thereof.

In some aspects, the present disclosure provides use of a compound of the present disclosure or a pharmaceutically acceptable salt thereof in the manufacture of a medicament for treating a Diabetes Mellitus in a subject in need thereof.

In some aspects, the present disclosure provides use of a compound of the present disclosure or a pharmaceutically acceptable salt thereof in the manufacture of a medicament for treating a Type 2 Diabetes Mellitus (T2D) in a subject in need thereof.

In some aspects, the present disclosure provides use of a compound of the present disclosure or a pharmaceutically acceptable salt thereof in the manufacture of a medicament for treating a Rasopathy in a subject in need thereof.

In some aspects, the present disclosure provides use of a compound of the present disclosure or a pharmaceutically acceptable salt thereof in the manufacture of a medicament for treating a Ovarian Cancer (OC) in a subject in need thereof.

The present disclosure provides compounds that function as inhibitors of PTPN1/PTPN2 (e.g., in vitro or in vivo). The present disclosure therefore provides a method of inhibiting of PTPN1/PTPN2 in vitro or in vivo, said method comprising contacting a cell with a therapeutically effective amount of a compound, or a pharmaceutically acceptable salt thereof, as defined herein.

In some embodiments, the inhibitors of PTPN1 or/and PTPN2 is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which PTPN1 or/and PTPN2 are implicated in a subject in need of such treatment, said method comprising administering to said subject a therapeutically effective amount of a compound, or a pharmaceutically acceptable salt thereof, or a pharmaceutical composition as defined herein.

In sore embodiments, the compounds described herein and compositions (e.g., compositions comprising a compound described herein) are used with a cancer immunotherapy (e.g., a checkpoint blocking antibody) to treat a subject (e.g., a human subject), e.g., suffering from a disease or disorder described herein (e.g., abnormal cell growth, e.g., cancer (e.g., a cancer described herein)). The methods described herein comprise administering a compound described herein and an immunotherapy to a subject having abnormal cell growth such as cancer.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:

| | |
|---|---|
| AcOH | acetic acid |
| aq. | aqueous |
| BnOH | benzyl alcohol |
| br. | broad |
| BSA | bovine serum albumin |
| conc. | concentrated |
| d | duplet |
| DCM | dichloromethane |
| DIPEA | N,N-diisopropylethylamine |
| DMEM | Dulbecco's modified eagle medium |
| DMSO | dimethyl sulfoxide |
| DTT | dithiothreitol |
| EDTA | ethylenediaminetetraacetic acid |
| ESI | electrospray ionization |
| FBS | fetal bovine serum |
| h | hour(s) |
| HEPES | 2-[4-(2-Hydroxyethyl)piperazin-1-yl]ethane-1-sulfonic acid |
| HPLC | high pressure (or performance) liquid chromatography |
| LCMS | liquid chromatography mass spectrometry |
| LC/MS/MS | liquid chromatography with tandem mass spectrometry |
| m | multiplet |
| M | molar |
| MCPBA | meta-Chloroperoxybenzoic acid |
| MHz | megahertz |
| min | minutes |
| NMR | nuclear magnetic resonance |
| Pd(cin)Cl- IPent^{An} | 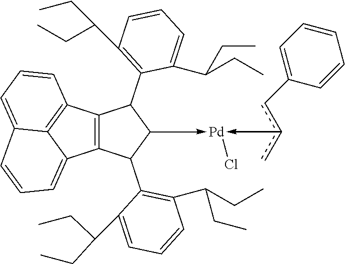 |
| q | quadruplet |
| rt | room temperature |
| s | singlet |
| sat. | saturated |
| t | temperature, triplet |
| TFA | trifluoroacetic acid |
| THF | tetrahydrofuran |
| TLC | thin layer chromatography |

### EXAMPLES

### General Synthetical Procedures and Examples of the Compound's Preparation

### Synthesis of Building Blocks

### Synthesis of 5-[2-benzyloxy-6-fluoro-4-(pyrimidin-2-ylamino)phenyl]-1,1-dioxo-1,2,5-thiadiazolidin-3-one (P1)

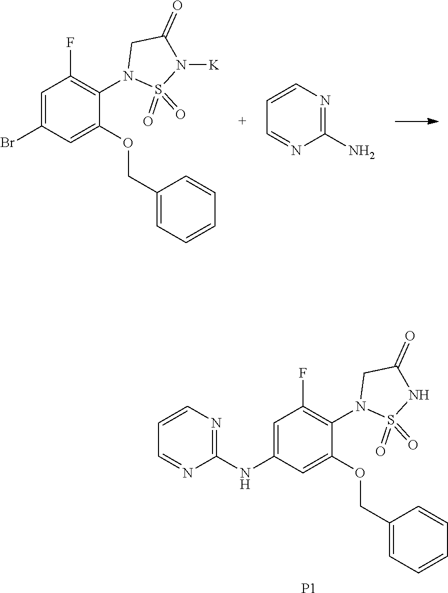

### Preparation 1. 5-[2-Benzyloxy-6-fluoro-4-(pyrimidin-2-ylamino)phenyl]-1,1-dioxo-1,2,5-thiadiazolidin-3-one (P1)

A suspension of pyrimidin-2-amine (53 mg, 0.55 mmol, 1 eq), [5-(2-benzyloxy-4-bromo-6-fluoro-phenyl)-1,1,3-trioxo-1,2,5-thiadiazolidin-2-yl]potassium (250 mg, 0.55 mmol, 1 eq), Cs₂CO₃ (360 mg, 1.1 mmol, 2 eq), XantPhos (32 mg, 0.055 mmol, 10% mol) and Pd₂dba₃ (25 mg, 0.027 mmol, 5% mol) was stirred under N₂ at 75° C. in dioxane (7 mL) for 12 h. A resulting solution was cooled to rt, acidified with 10% aqueous H₃PO₄ and extracted with EtOAc (3×10 mL). The combined organic phases were washed with brine, dried with Na₂SO₄ and then all volatiles were concentrated under reduced pressure. The residue was dissolved in MeOH and filtered through a small pad of Celite. The solvent was removed in vacuum, and the residue was used on the next step without further purification (100 mg of crude, 40%). LCMS (ESI⁺) m/z: 431 [M+H]⁺.

### Synthesis of 5-[2-benzyloxy-6-fluoro-4-[(4-methylpyrimidin-2-yl)amino]phenyl]-1,1-dioxo-1,2,5-thiadiazolidin-3-one (P2)

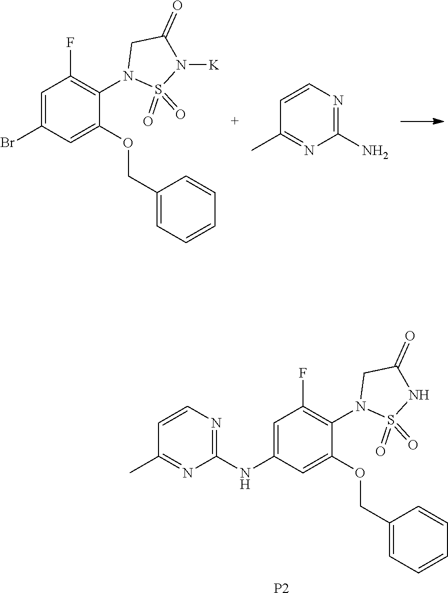

### Preparation 2. 5-[2-Benzyloxy-6-fluoro-4-[(4-methylpyrimidin-2-yl)amino]phenyl]-1,1-dioxo-1,2,5-thiadiazolidin-3-one (P2)

A suspension of 4-methylpyrimidin-2-amine (120 mg, 1.1 mmol, 1 eq), [5-(2-benzyloxy-4-bromo-6-fluoro-phenyl)-1,1,3-trioxo-1,2,5-thiadiazolidin-2-yl]potassium (500 mg, 1.1 mmol, 1 eq), Cs₂CO₃ (720 mg, 2.2 mmol, 2 eq), XantPhos (64 mg, 0.11 mmol, 10% mol) and Pd₂dba₃ (50 mg, 0.055 mmol, 5% mol) was stirred under N₂ at 75° C. in dioxane (20 mL) for 12 h. A resulting solution was cooled to rt, acidified with 10% aqueous H₃PO₄ and extracted with EtOAc (3×10 mL). The combined organic phases were washed with brine, dried with Na₂SO₄ and then all volatiles were concentrated under reduced pressure. The residue was dissolved in MeOH and filtered through a small pad of Celite. The solvent was removed in vacuum, and the residue was used on the next step without further purification (140 mg of crude, 26%). LCMS (ESI⁺) m/z: 444 [M+H]⁺.

### Synthesis of 5-[2-benzyloxy-4-[(5-chloropyrimidin-2-yl)amino]-6-fluoro-phenyl]-1,1-dioxo-1,2,5-thiadiazolidin-3-one (P3)

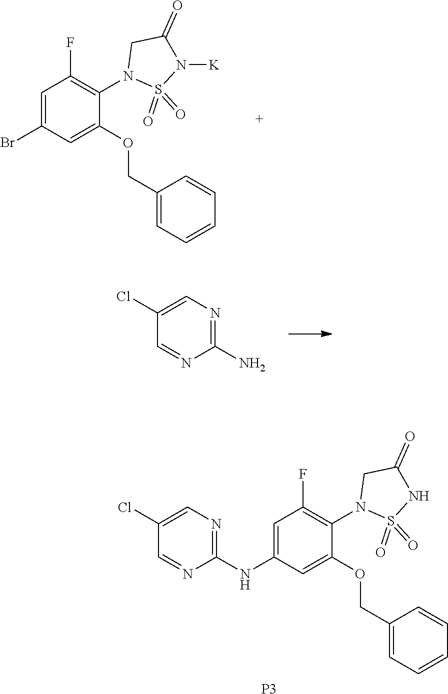

### Preparation 3. 5-[2-Benzyloxy-4-[(5-chloropyrimidin-2-yl)amino]-6-fluoro-phenyl]-1,1-dioxo-1,2,5-thiadiazolidin-3-one (P3)

A suspension of 5-chloropyrimidin-2-amine (90 mg, 0.7 mmol, 1 eq), [5-(2-benzyloxy-4-bromo-6-fluoro-phenyl)-1,1,3-trioxo-1,2,5-thiadiazolidin-2-yl]potassium (285 mg, 0.63 mmol, 1 eq), tert-BuONa (180 mg, 1.88 mmol, 3 eq), XantPhos (35 mg, 0.06 mmol, 10% mol) and Pd₂dba₃ (30 mg, 0.033 mmol, 5% mol) was stirred under N₂ at 75° C. in dioxane (20 mL) for 4 h. A resulting solution was cooled to rt, acidified with 10% aqueous H₃PO₄ and extracted with EtOAc (3×10 mL). The combined organic phases were washed with brine, dried with Na₂SO₄ and then all volatiles were concentrated under reduced pressure. The residue was dissolved in mixture of EtOAc/MeOH=5/1 and filtered through a small pad of silica. The solvent was removed in vacuum, and the residue was used on the next step without further purification (170 mg of crude P3, 61%). LCMS (ESI⁺) m/z: 464 [M+H]⁺.

### Synthesis of 5-[2-benzyloxy-4-[(4-benzyloxy-5-methyl-pyrimidin-2-yl)amino]-6-fluoro-phenyl]-1,1-dioxo-1,2,5-thiadiazolidin-3-one (P6)

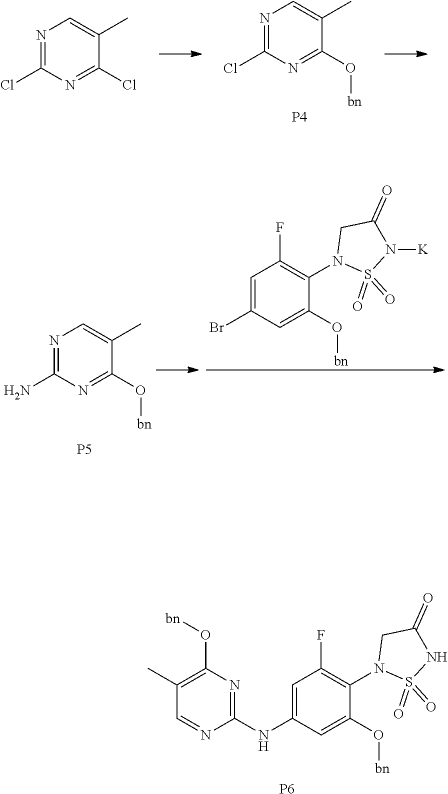

### Preparation 4. 4-Benzyloxy-2-chloro-5-methyl-pyrimidine (P4)

A suspension of 2,4-dichloro-5-methyl-pyrimidine (326 mg, 2 mmol, 1 eq), BnOH (216 mg, 2 mmol, 1 eq) and Cs₂CO₃ (1.4 g, 4.4 mmol, 2.2 eq) was stirred in a dry MeCN (20 mL) at rt for 12 h. Precipitate was filtered off, the solvent was removed in vacuum and the residue was purified on silica (Hex/DCM=1/5) to give 4-benzyloxy-2-chloro-5-methyl-pyrimidine (P4, 440 mg, 93%). ¹H NMR (400 MHz, CDCl₃), δ: 8.14 (s, 1H), 7.65-7.24 (m, 5H), 5.45 (d, J=15.7 Hz, 2H), 2.16 (s, 3H). LCMS (ESI⁺) m/z: 236 [M+H]⁺.

### Preparation 5. 4-Benzyloxy-2-amino-5-methyl-pyrimidine (P5)

A suspension of 4-benzyloxy-2-chloro-5-methyl-pyrimidine (P4, 440 mg, 1.87 mmol, 1 eq), benzophenone imine (340 mg, 1.87 mmol, 1 eq), tert-BuONa (270 mg, 2.81 mmol, 1.5 eq) and Pd(cin)Cl-IPent^{An} (50 mg, 0.06 mmol, 3% mol) was stirred under N₂ at 75° C. in dioxane (7 mL) for 12 h. A resulting solution was cooled to rt, and conc. aqueous HCl (1 mL) was added. The mixture was stirred for 30 min, and then all volatiles were concentrated under reduced pressure. The residue was diluted with 10% aqueous HCl (5 mL) and washed with EtOAc (2×10 mL). Aqueous acidic layer was basified to pH 9 with Na₂CO₃ and extracted with EtOAc (3×10 mL). The solvent was removed in vacuum, and the residue was purified on silica (Hex/EtOAc=1/5) to obtain 4-benzyloxy-2-amino-5-methyl-pyrimidine (P5, 290 mg, 72%). ¹H NMR (400 MHz, DMSO-d₆), δ: 7.82 (s, 1H), 7.32-7.35 (m, 5H), 6.27 (s, 2H), 5.34 (s, 2H), 1.92 (s, 3H). LCMS (ESI⁺) m/z: 216 [M+H]⁺.

### Preparation 6. 5-[2-Benzyloxy-4-[(4-benzyloxy-5-methyl-pyrimidin-2-yl)amino]-6-fluoro-phenyl]-1,1-dioxo-1,2,5-thiadiazolidin-3-one (P6)

A suspension of 4-benzyloxy-2-amino-5-methyl-pyrimidine (P5, 142 mg, 0.66 mmol, 1 eq), [5-(2-benzyloxy-4-bromo-6-fluoro-phenyl)-1,1,3-trioxo-1,2,5-thiadiazolidin-2-yl]potassium (250 mg, 0.55 mmol, 1 eq), Cs₂CO₃ (360 mg, 1.1 mmol, 2 eq), XantPhos (32 mg, 0.055 mmol, 10% mol) and Pd₂dba₃ (25 mg, 0.027 mmol, 5% mol) was stirred under N₂ at 75° C. in dioxane (7 mL) for 12 h. A resulting solution was cooled to rt, acidified with 10% aq. H₃PO₄ and extracted with EtOAc (3×10 mL). The combined organic phases were washed with brine, dried with Na₂SO₄ and then all volatiles were concentrated under reduced pressure. The residue was dissolved in MeOH and filtered through a small pad of Celite. The solvent was removed in vacuum, and the residue (P6) was used on the next step without further purification (300 mg of crude, 98%). LCMS (ESI⁺) m/z: 550 [M+H]⁺.

### Synthesis of 5-[2-benzyloxy-4-[(4-benzyloxypyrimidin-2-yl)amino]-6-fluoro-phenyl]-1,1-dioxo-1,2,5-thiadiazolidin-3-one (P9)

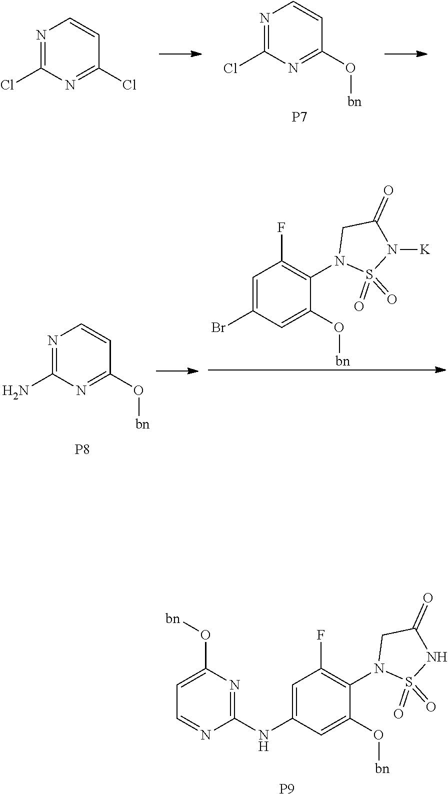

### Preparation 7. 4-Benzyloxy-2-chloropyrimidine (P7)

A suspension of 2,4-dichloropyrimidine (1000 mg, 6.7 mmol, 1 eq), BnOH (725 mg, 6.7 mmol, 1 eq) and Cs₂CO₃ (4.8 g, 14.7 mmol, 2.2 eq) was stirred in a dry MeCN (100 mL) at rt for 12 h. Precipitate was filtered off, the solvent was removed in vacuum and the residue was purified on silica using DCM as eluent to give 4-benzyloxy-2-chloropyrimidine (P7, 1150 mg, 78%). ¹H NMR (400 MHz, DMSO-d₆), δ: 8.48 (d, J=5.7 Hz, 1H), 7.63-7.23 (m, 5H), 7.04 (d, J=5.7 Hz, 1H), 5.41 (s, 2H). LCMS (ESI⁺) m/z 221 [M+H]⁺.

### Preparation 8. 4-Benzyloxypyrimidin-2-amine (P8)

A suspension of 4-benzyloxy-2-chloropyrimidine (P7, 600 mg, 2.73 mmol, 1 eq), benzophenone imine (495 mg, 2.73 mmol, 1 eq), NaOt-Bu (525 mg, 5.47 mmol, 2 eq) and Pd(cin)Cl-IPent^{An} (72 mg, 0.08 mmol, 3% mol) was stirred under N₂ at 75° C. in dioxane (14 mL) for 12 h. A resulting solution was cooled to rt, and conc. aq. HCl (2 mL) was added. The mixture was stirred for 30 min, and then all volatiles were concentrated under reduced pressure. The residue was diluted with 10% aq. HCl (10 mL) and washed with EtOAc (2×15 mL). Aqueous acidic layer was basified to pH 9 with Na₂CO₃ and extracted with EtOAc (3×15 mL). The solvent was removed in vacuum, and the residue was purified on silica (Hex/EtOAc=1/5) to obtain 4-benzyloxypyrimidin-2-amine (P8, 260 mg, 47%). LCMS (ESI⁺) m/z: 202 [M+H]⁺.

### Preparation 9. 5-[2-Benzyloxy-4-[(4-benzyloxypyrimidin-2-yl)amino]-6-fluoro-phenyl]-1,1-dioxo-1,2,5-thiadiazolidin-3-one (P9)

A suspension of 4-benzyloxypyrimidin-2-amine (P8, 88 mg, 0.44 mmol, 1 eq), [5-(2-benzyloxy-4-bromo-6-fluoro-phenyl)-1,1,3-trioxo-1,2,5-thiadiazolidin-2-yl]potassium (200 mg, 0.44 mmol, 1 eq), Cs₂CO₃ (290 mg, 0.89 mmol, 2 eq), XantPhos (25 mg, 0.044 mmol, 10% mol) and Pd₂dba₃ (20 mg, 0.022 mmol, 5% mol) was stirred under N₂ at 75° C. in dioxane (7 mL) for 12 h. A resulting solution was cooled to rt, acidified with 10% aqueous H₃PO₄ and extracted with EtOAc (3×10 mL) The combined organic phases were washed with brine, dried with Na₂SO₄ and then all volatiles were concentrated under reduced pressure. The residue was dissolved in mixture of EtOAc/MeOH=5/1 and filtered through a small pad of silica. The solvent was removed in vacuum, and the residue was used on the next step without further purification (100 mg of crude P9, 43%). LCMS (ESI⁺) m/z: 537 [M+H]⁺.

### Synthesis of 5-[2-benzyloxy-4-[(4-benzyloxyquinazolin-2-yl)amino]-6-fluoro-phenyl]-1,1-dioxo-1,2,5-thiadiazolidin-3-one (P12)

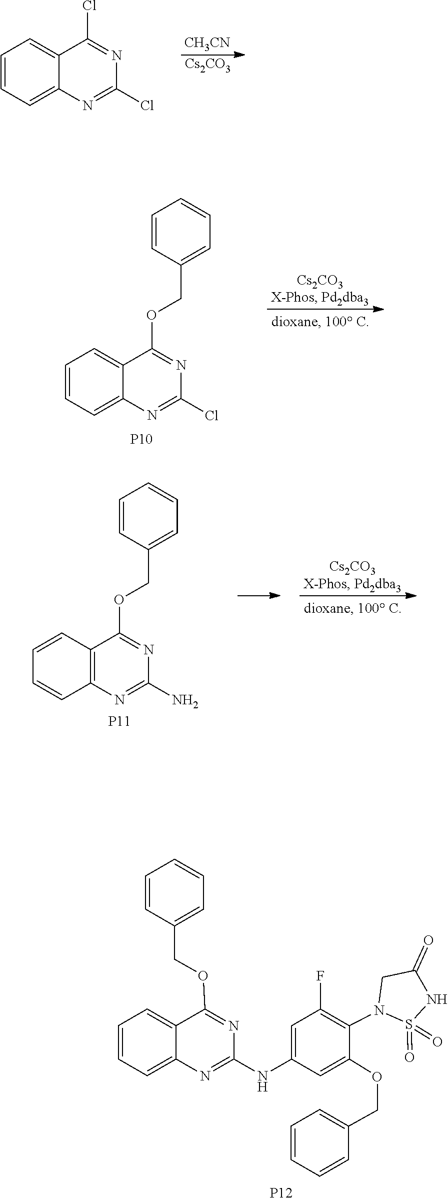

### Preparation 10. 4-Benzyloxy-2-chloro-quinazoline (P10)

1 g (5 mmol) of 2,4-dichloroquinazoline, 0.59 g (5.5 mmol) benzyl alcohol and 3.2 g (10 mmol) of Cs₂CO₃ was dissolved in 10 ml acetonitrile and stirred at rt for 72 h. Water (20 mL) was added to the reaction mixture and stirred for 1 h. The precipitate was filtered, washed with water, hexane and dried to yield 1.2 g (82%) of title compound—P10. ¹H NMR (400 MHz, DMSO-d₆), δ: 8.18 (d, 1H), 8.0 (d, 1H), 7.89 (d, 1H), 7.7 (t, 1H), 7.58 (d, 2H), 7.39-7.46 (m, 3H), 5.64 (s, 2H).

### Preparation 11. 4-Benzyloxyquinazolin-2-amine (P11)

4-Benzyloxy-2-chloro-quinazoline (P10, 0.8 g, 2.9 mmol), tert-butylamine (0.69 g, 5.9 mmol) and Cs₂CO₃ (1.9 g, 5.9 mmol) were mixed with 25 ml of dioxane and stirred in the steam of N₂ for 15 min. X-Phos (0.12 g, 0.3 mmol) and Pd₂dba₃ (0.27 g, 0.3 mmol) were added to the reaction mixture and stirred at 100° C. under atmosphere of N₂ overnight. 25 ml EtOAc was added to the reaction mixture with cooling and formed precipitate was removed by filtration and the solution was evaporated to dryness. Residue was purified by column chromatography on silica gel eluting with CH₂Cl₂/methanol (30:1) to give the title compound P11 (0.4 g, 54%). ¹H NMR (400 MHz, DMSO-d₆), δ: 7.86 (d, 1H), 7.61 (t, 1H), 7.54 (d, 2H), 7.33-7.43 (m, 5H), 6.67 (br. s, 2H), 5.54 (s, 2H). LCMS (ESI⁺) m/z: 252 [M+H]⁺.

### Preparation 12. 5-[2-Benzyloxy-4-[(4-benzyloxyquinazolin-2-yl)amino]-6-fluoro-phenyl]-1,1-dioxo-1,2,5-thiadiazolidin-3-one (P12)

4-Benzyloxyquinazolin-2-amine (P11, 0.4 g, 1.6 mmol), 5-[2-(benzyloxy)-4-bromo-6-fluorophenyl]-1,2,5-thiadiazolidin-3-one 1,1-dioxide (0.6 g, 1.3 mmol) and Cs₂CO₃ (0.86 g, 2.6 mmol) were dissolved in 15 ml of dioxane and stirred in the steam N₂ for 15 min. X-Phos (0.054 g, 0.1 mmol) and Pd₂dba₃ (0.12 g, 0.13 mmol) was added to the reaction mixture and stirred at 100° C. in the steam of N₂ overnight. 25 ml of EtOAc was added to the reaction mixture after it was cooled, and the precipitate was filtered off Filtrate was evaporated to dryness; residue was purified be column chromatography on silica gel eluting with ethyl acetate/methanol (4:1) to give P12 (0.5 g, 64%). ¹H NMR (400 MHz, DMSO-d₆), δ: 9.78 (br. s, 1H), 7.99 (d, 1H), 7.77 (t, 1H), 7.7 (br. s., 1H), 7.53-7.63 (m, 4H), 7.29-7.43 (m, 10H), 5.87 (s, 2H), 5.18 (s, 2H), 3.96 (s, 2H). LCMS (ESI⁺) m/z: 586 [M+H]⁺.

### Synthesis of 5-[2-benzyloxy-4-[(4-benzyloxy-6-methoxy-pyrimidin-2-yl)amino]-6-fluoro-phenyl]-1,1-dioxo-1,2,5-thiadiazolidin-3-one (P14)

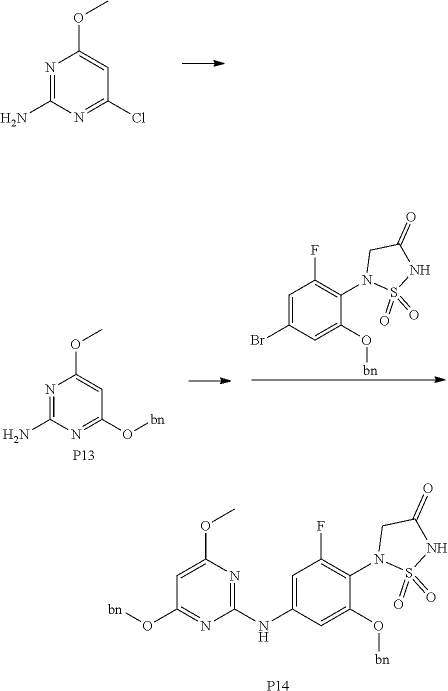

### Preparation 13. 4-Benzyloxy-6-methoxy-pyrimidin-2-amine (P13)

To a stirred solution of BnOH (380 mg, 3.52 mmol, 1.1 eq) in a dry THF (20 mL) NaH (60% in mineral oil, 300 mg, 7.5 mmol, 2.5 eq) was added at rt. This mixture was stirred for 30 min, then 4-chloro-6-methoxy-pyrimidin-2-amine (480 mg, 3 mmol, 1 eq) was added in one portion. The resulting suspension was stirred at reflux for 12 h, cooled to rt, and the excess of NaH was quenched with H₂O (1 mL). All volatiles were removed under reduced pressure, and the residue was purified on silica (Hex/EtOAc=3/1) to give 4-benzyloxy-6-methoxy-pyrimidin-2-amine (P13, 412 mg, 60%). ¹H NMR (400 MHz, DMSO-d₆), δ: 7.36 (dq, J=21.5, 6.9 Hz, 5H), 6.58 (s, 2H), 5.27 (s, 2H), 3.76 (s, 3H). LCMS (ESI⁺) m/z: 232 [M+H]⁺.

### Preparation 14. 5-[2-Benzyloxy-4-[(4-benzyloxy-6-methoxy-pyrimidin-2-yl)amino]-6-fluoro-phenyl]-1,1-dioxo-1,2,5-thiadiazolidin-3-one (P14)

A suspension of 4-benzyloxy-6-methoxy-pyrimidin-2-amine (P13, 125 mg, 0.54 mmol, 1 eq), [5-(2-benzyloxy-4-bromo-6-fluoro-phenyl)-1,1,3-trioxo-1,2,5-thiadiazolidin-2-yl]potassium (250 mg, 0.55 mmol, 1 eq), Cs₂CO₃ (360 mg, 1.1 mmol, 2 eq), XantPhos (32 mg, 0.055 mmol, 10% mol) and Pd₂dba₃ (25 mg, 0.027 mmol, 5% mol) was stirred under N₂ at 75° C. in dioxane (7 mL) for 12 h. A resulting solution was cooled to rt, acidified with 10% aqueous H₃PO₄ and extracted with EtOAc (3×10 mL) The combined organic phases were washed with brine, dried with Na₂SO₄ and then all volatiles were concentrated under reduced pressure. The residue was dissolved in MeOH and filtered through a small pad of Celite. The solvent was removed in vacuum, and the residue was used on the next step without further purification (P14, 210 mg of crude, 65%). LCMS (ESI⁺) m/z: 566 [M+H]⁺.

### Synthesis of 5-[2-benzyloxy-4-[(4-benzyloxy-6-methyl-pyrimidin-2-yl)amino]-6-fluoro-phenyl]-1,1-dioxo-1,2,5-thiadiazolidin-3-one (P21)

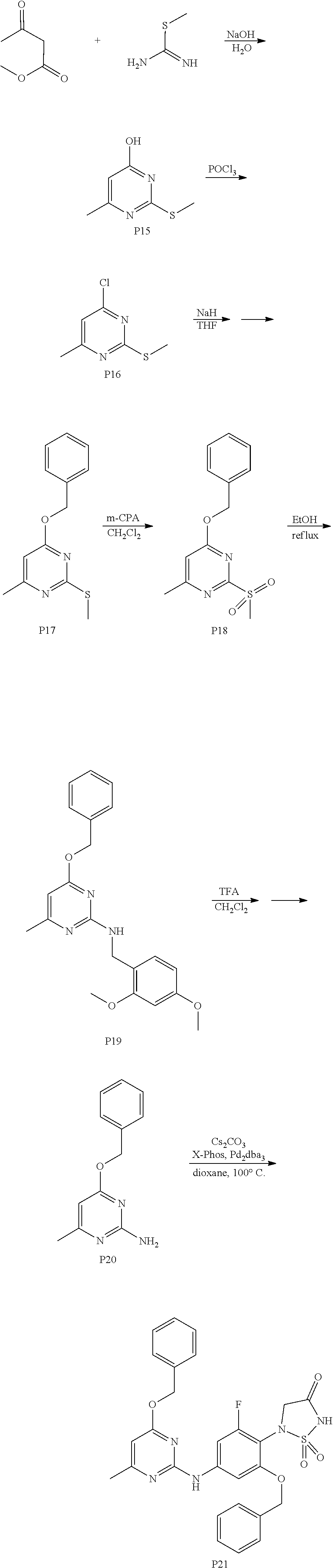

### Preparation 15. 6-Methyl-2-methylsulfanyl-pyrimidin-4-ol (P15)

A solution of 6.1 g (0.15 mol) of sodium hydroxide in 50 ml of water was added dropwise at room temperature to a mixture of 10 g (0.077 moles) of acetoacetic acid ethyl ester, 11.7 g (0.042 mol) of S-methyl-iso-thiourea sulfate and 30 ml of water and the batch was allowed to react for a further 18 h at rt. The reaction mixture was then acidified with acetic acid and the product which had crystallized out was filtered off and rinsed thoroughly with water to yield 8 g (66%) of title compound P15. ¹H NMR (400 MHz, DMSO-d₆), δ: 5.94 (s, 1H), 2.46 (s, 3H(SCH₃)), 2.16 (s, 3H(CH₃)).

### Preparation 16. 4-Chloro-6-methyl-2-methylsulfanyl-pyrimidine (P16)

6-Methyl-2-methylsulfanyl-pyrimidin-4-ol (P15, 3.5 g, 0.022 mol) and phosphorous oxychloride (20 mL) were combined and refluxed for 3 h. The reaction mixture was cooled to rt and poured onto crushed ice. The resultant aqueous mixture was extracted with ethyl acetate and organic layer was washed with saturated aq. sodium bicarbonate followed by a water wash, dried over magnesium sulfate, and dried in vacuum to give of title compound P16 (2.27 g, 69%). ¹H NMR (400 MHz, CDCl₃), δ: 6.85 (s, 1H), 2.55 (s, 3H (SCH₃)), 2.43 (s, 3H (CH₃)). LCMS (ESI⁺) m/z: 175 [M+H]⁺.

### Preparation 17. 4-Benzyloxy-6-methyl-2-methylsulfanyl-pyrimidine (P17)

To a stirred suspension of sodium hydride (0.74 g, 60% dispersion in mineral oil) in tetrahydrofuran (5 ml) at 0° C. was added benzyl alcohol (1.77 g, 0.016 mol). The mixture was stirred for 15 min then a solution of 4-chloro-6-methyl-2-methylsulfanyl-pyrimidine (P16, 2.6 g, 0.015 mol) in THF (10 ml) was added dropwise. The reaction was allowed to warm to rt and stirred for 8 h. Solvent was evaporated under reduced pressure and the remaining residue was dissolved in DCM (50 ml) and washed with water (2×50 ml). Organics were separated, dried over sodium sulfate and the solvent was removed under reduced pressure and residue was purified by flash chromatography on silica gel eluting with hexane/DCM (2:1) to yield 3.5 g (95%) of title compound—P17. ¹H NMR (400 MHz, DMSO-d₆), δ: 7.44-7.32 (m, 5H), 6.53 (s, 1H), 5.39 (s, 2H), 2.48 (s, 3H), 2.31 (m, 2H). LCMS (ESI⁺) m/z: 247 [M+H]⁺.

### Preparation 18. 4-Benzyloxy-6-methyl-2-methylsulfonyl-pyrimidine (P18)

4-Benzyloxy-6-methyl-2-methylsulfanyl-pyrimidine (P17, 0.6 g, 2.4 mmol) was initially charged in 25 ml of DCM, and 3-chloroperbenzoic acid (1.4 g, 6 mmol) was added at rt. After 10 h, the reaction was diluted with DCM and washed twice with 1M aq. sodium hydroxide solution, then with saturated aq. sodium sulfite solution and finally with water. The organic phase was dried over sodium sulfate, filtered and the solvent was removed under reduced pressure. The residue was purified by flash chromatography on silica gel eluting with DCM to yield 0.65 g (95%) of title compound—P18. ¹H NMR (400 MHz, DMSO-d₆), δ: 7.50-7.36 (m, 5H), 7.15 (s, 1H), 2.55 (s, 3H), 2.41 (s, 3H), 1.81-1.70 (m, 2H). LCMS (ESI⁺) m/z: 279 [M+H]⁺.

### Preparation 19. 4-benzyloxy-N-[(2,4-dimethoxyphenyl)methyl]-6-methyl-pyrimidin-2-amine (P19)

0.25 g (0.8 mmol) of 4-benzyloxy-6-methyl-2-methylsulfonyl-pyrimidine (P18) and 0.22 g (1.3 mmol) of 2,4-dimethoxybenzyl amine was dissolved in 10 ml of ethanol and refluxed for 72 h, the reaction mixture was concentrated under reduced pressure. Residue was purified by flash chromatography on silica gel eluting with DCM/ethyl acetate (7:1) to yield 0.3 g (91%) of P19. ¹H NMR (400 MHz, DMSO-d₆), δ: 2.13 (s, 3H), 1.40 (s, 9H), 1.7-1.81 (m, 2H), 2.86-2.93 (m, 2H), 3.72 (s, 3H), 3.78 (s, 3H), 3.89 (d, 2H), 5.28 (s, 2H), 5.91 (s, 1H), 6.43 (d, 1H), 6.53 (s, 1H), 7.08 (d, 1H), 7.25-7.55 (m, 5H). LCMS (ESI⁺) m/z: 366 [M+H]⁺.

### Preparation 20. 4-Benzyloxy-6-methyl-pyrimidin-2-amine (P20)

4-Benzyloxy-N-[(2,4-dimethoxyphenyl)methyl]-6-methyl-pyrimidin-2-amine (P19, 0.3 g, 0.8 mmol) was dissolved in 10 ml of DCM and 1 g (8.77 mmol) TFA was added to the solution. The reaction mixture was stirred at rt for 10 h. After completing the reaction to the reaction mixture was added 5.6 g (41 mmol) of potassium carbonate and 1 ml of water, the organic phase was separated and concentrated under reduced pressure. The residue was used in the next step without additional purification. Yield of P20—0.15 g (85%). ¹H NMR (400 MHz, DMSO-d₆), δ: 2.3 (s, 3H), 5.42 (s, 2H), 7.37-7.48 (m, 5H), 8.1-8.5 (br. s, 2H). LCMS (ESI⁺) m/z: 216 [M+H]⁺.

### Preparation 21. 5-[2-Benzyloxy-4-[(4-benzyloxy-6-methyl-pyrimidin-2-yl)amino]-6-fluoro-phenyl]-1,1-dioxo-1,2,5-thiadiazolidin-3-one (P21)

4-Benzyloxy-6-methyl-pyrimidin-2-amine (P20, 0.15 g, 0.66 mmol), 5-[2-(benzyloxy)-4-bromo-6-fluorophenyl]-1,2,5-thiadiazolidin-3-one-1,1-dioxide (0.26 g, 0.55 mmol), and Cs₂CO₃ (0.375 g, 1.2 mmol) were dissolved in 10 ml of dioxane and stirred in the steam of N₂ for 15 min. X-Phos (0.034 g, 0.06 mmol) and Pd₂dba₃ (0.06 g, 0.06 mol) were added to the reaction mixture and stirred at 100° C. in the steam of N₂ for overnight. 15 mL of EtOAc was added to the reaction mixture after it was cooled, and the precipitate was removed by filtration and the solution was evaporated to dryness. Residue was purified by column chromatography on silica gel eluting with ethyl acetate/methanol (4:1) to give 0.15 g (47%) of the title compound—P21. ¹H NMR (400 MHz, DMSO-d₆), δ: 2.29 (s, 3H), 3.94 (s, 2H), 5.10 (s, 2H), 5.45 (s, 2H), 6.28 (s, 1H), 7.23-7.42 (m, 8H), 7.44-7.49 (m, 4H), 7.53 (br. s, 1H), 9.71 (s, 1H). LCMS (ESI⁺) m/z: 550 [M+H]⁺.

### Synthesis of 5-[2-benzyloxy-4-[(4-benzyloxy-6-chloro-5-isobutyl-pyrimidin-2-yl)amino]-6-fluoro-phenyl]-1,1-dioxo-1,2,5-thiadiazolidin-3-one (P28)

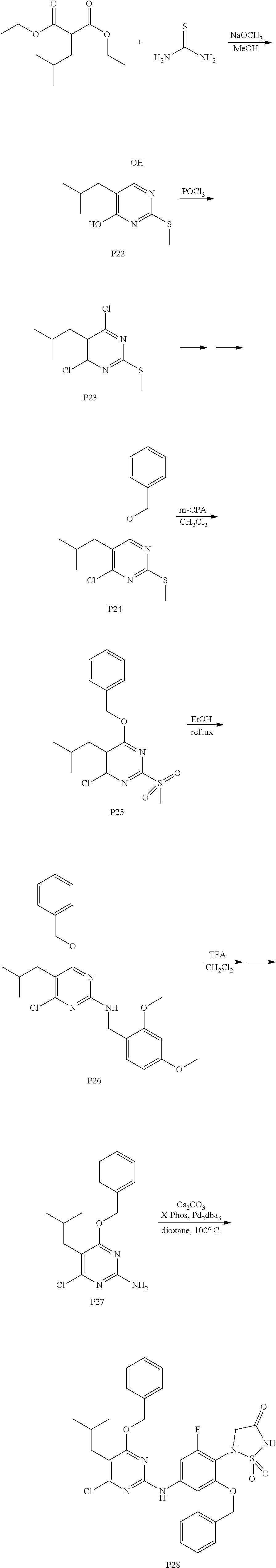

### Preparation 22. 5-iso-Butyl-2-methylsulfanyl-pyrimidine-4,6-diol (P22)

To a solution of thiourea (3.2 g, 42 mmol) in 50 ml of MeOH was slowly added NaOMe (2.27 g, 42 mmol) and the mixture was stirred for 10 min. Subsequently, a solution of diethyl isopropylmalonate (9 g, 42 mmol) in 50 ml of MeOH was added dropwise and stirred continued overnight. Additional amount of NaOMe (4.6 g, 84 mmol) was added and the reaction mixture was heated at reflux for 6 h. Upon cooling down to rt, iodomethane (1.3 ml, 42 mmol) was added and stirring was continued overnight. Solvent was removed under reduced pressure to afford a solid residue. The residue was dissolved in water (100 ml) and the solution was acidified with conc. HCl, as a result a precipitate was formed. The precipitate was collected and washed with water (3×25 ml), dried to afford the desired product (P22) as a yellow solid (3 g, 95%). ¹H NMR (400 MHz, DMSO-d₆), δ: 0.8 (s, 3H), 0.82 (s, 3H), 1.75-1.87 (m, 1H), 2.12 (d, 2H), 2.47 (s, 3H), 6.19 (br. s, 2H).

### Preparation 23. 4,6-Dichloro-5-isobutyl-2-methylsulfanyl-pyrimidine (P23)

A mixture of 5-isobuthyl-2-methylsulfanyl-pyrimidin-4,6-diol (P22, 3 g, 0.014 mol), phosphorous oxychloride (25 mL) and DIPEA (2.35 g, 0.018 mol) was refluxed for 3 h. The reaction mixture was cooled to rt and poured onto crushed ice. Then the mixture was extracted with ethyl acetate and the organic layer was washed with sat. aq. sodium bicarbonate followed by a water wash, dried over magnesium sulfate, and dried in vacuum to give the title compound—P23 (3.1 g, 88%). ¹H NMR (400 MHz, CDCl₃), δ: 0.99 (s, 3H), 1.00 (s, 3H), 2.04-2.14 (m, 1H), 2.57 (s, 3H), 2.71 (d, 2H). LCMS (ESI⁺) m/z: 252 [M+H]⁺.

### Preparation 24. 4-Benzyloxy-6-chloro-5-isobutyl-2-methylsulfanyl-pyrimidine (P24)

To a stirred suspension of sodium hydride (0.32 g, 60% dispersion in mineral oil) in THF (5 ml) at 0° C. was added benzyl alcohol (0.73 g, 6.77 mmol). The mixture was stirred for 15 minutes then a solution of 4.6-dichloro-5-isobuthyl-2-methylsulfanyl-pyrimidine (P23, 1.7 g, 6.77 mmol) in THF (10 ml) was added dropwise. The reaction was allowed to warm to rt and stirred for additional 3 h. Solvent was evaporated under reduced pressure and the remaining residue dissolved in DCM (50 ml) and washed with water (2×50 ml). Organics were separated, dried over sodium sulfate and the solvent was removed under reduced pressure. Residue was purified by flash chromatography on silica gel eluting with hexane/DCM (2:1) to give 1.4 g (64%) of the title compound P24. ¹H NMR (400 MHz, DMSO-d₆), δ: 0.85 (s, 3H), 0.87 (s, 3H), 1.87-1.97 (m, 1H), 2.43-2.49 (m, 5H), 5.45 (s, 2H), 7.32-7.46 (m, 5H). LCMS (ESI⁺) m/z: 323 [M+H]⁺.

### Preparation 25. 4-Benzyloxy-6-chloro-5-isobutyl-2-methylsulfonyl-pyrimidine (P25)

4-Benzyloxy-6-chloro-5-isobutyl-2-methylsulfanyl-pyrimidine (P24, 1.4 g, 4.3 mmol) was initially charged in 25 ml of DCM, and 3-chloroperbenzoic acid (2.49 g, 108 mmol) was added at rt. After 10 h, the reaction was diluted with DCM and washed twice with 1M aq. sodium hydroxide solution, then with saturated aqueous sodium sulfite solution and finally with water. The organic phase was dried over sodium sulfate, filtered and the solvent was removed under reduced pressure. The residue was purified by flash chromatography on silica gel eluting with DCM to give 1.3 g (84%) of the title compound—P25. ¹H NMR (400 MHz, CDCl₃), δ: 0.94 (s, 3H), 0.95 (s, 3H), 1.98-2.10 (m, 1H), 2.67 (d, 2H), 3.33 (s, 3H) 5.55 (s, 2H) 7.39-7.47 (m, 5H). LCMS (ESI⁺) m/z: 355 [M+H]⁺.

### Preparation 26. 4-Benzyloxy-6-chloro-N-[(2,4-dimethoxyphenyl)methyl]-5-isobutyl-pyrimidin-2-amine (P26)

4-Benzyloxy-6-chloro-5-isobuthyl-2-methylsulfonyl-pyrimidine (P25, 1.2 g, 3.3 mmol) and 2,4-dimethoxybenzyl amine (0.84 g, 5 mmol) were dissolved in 10 ml of ethanol and refluxed for 72 h, the reaction mixture was concentrated under reduced pressure. The residue was purified by flash chromatography on silica gel eluting with DCM/ethyl acetate (7:1) to yield the compound P26 (1.1 g, 73.6%). ¹H NMR (400 MHz, DMSO-d₆), δ: 0.82 (s, 3H), 0.83 (s, 3H), 1.83-1.90 (m, 1H), 2.33 (d, 2H), 3.73 (s, 3H), 3.78 (s, 3H), 4.34 (s, 2H), 5.33 (s, 2H), 6.44 (s, 1H), 6.54 (d, 1H), 7.08 (d, 1H) 7.25-7.47 (m, 5H). LCMS (ESI⁺) m/z: 443 [M+H]⁺.

### Preparation 27. 4-Benzyloxy-6-chloro-5-isobutyl-pyrimidin-2-amine (P28)

4-Benzyloxy-6-chloro-N-[(2,4-dimethoxyphenyl)methyl]-5-isobuthyl-pyrimidine-2-amine (P26, 1.1 g, 2.4 mmol) was dissolved in 10 ml of DCM and 2.8 g (24.8 mmol) of trifluoroacetic acid was added to the solution. The reaction mixture was stirred at rt for 3 h. After completing of the reaction 25 g (0.18 mol) potassium carbonate and 2 ml of water were added to reaction mixture, the organic phase was separated and concentrated under reduced pressure. Obtained residue was used in the next step without additional purification. The yield of P27—0.7 g (96%). ¹H NMR (400 MHz, DMSO-d₆), δ: 0.82 (s, 3H), 0.84 (s, 3H), 1.77-1.89 (m, 1H), 2.35 (d, 2H), 5.33 (s, 2H), 6.82 (br. s, 2H), 7.30-7.46 (m, 5H). LCMS (ESI⁺) m/z: 292 [M+H]⁺.

### Preparation 28. 5-[2-Benzyloxy-4-[(4-benzyloxy-6-chloro-5-isobutyl-pyrimidin-2-yl)amino]-6-fluoro-phenyl]-1,1-dioxo-1,2,5-thiadiazolidin-3-one (P28)

4-Benzyloxy-6-chloro-5-isobutyl-pyrimidin-2-amine (P27, 0.46 g, 1.5 mmol), 5-[2-(benzyloxy)-4-bromo-6-fluorophenyl]-1,2,5-thiadiazolidin-3-one-1,1-dioxide (0.6 g, 1.3 mmol), and Cs₂CO₃ (0.86 g, 2.6 mmol) were dissolved in 15 ml of dioxane and stirred in the steam of N₂ for 15 min. X-Phos (0.076 g, 0.13 mmol) and Pd₂dba₃ (0.12 g, 0.13 mol) were added to the reaction mixture and stirred at 100° C. in the steam of N₂ overnight. 15 mL of EtOAc was added to the reaction mixture after it was cooled and formed precipitate was filtered off and filtrate was evaporated to dryness. Residue was purified by column chromatography on silica gel eluting with ethyl acetate/methanol (4:1) to give the title compound P28 (0.65 g, 78%). ¹H NMR (400 MHz, DMSO-d₆), δ: 0.87 (s, 3H), 0.88 (s, 3H), 1.86-1.95 (m, 1H), 2.45 (d, 2H), 4.04 (s, 2H), 5.11 (s, 2H), 5.51 (s, 2H), 7.15-7.55 (m, 13H) 10.05 (s, 1H). LCMS (ESI⁺) m/z: 627 [M+H]⁺.

### Synthesis of 5-[2-benzyloxy-4-[(4,6-dibenzyloxy-5-isobutyl-pyrimidin-2-yl)amino]-6-fluoro-phenyl]-1,1-dioxo-1,2,5-thiadiazolidin-3-one (P33)

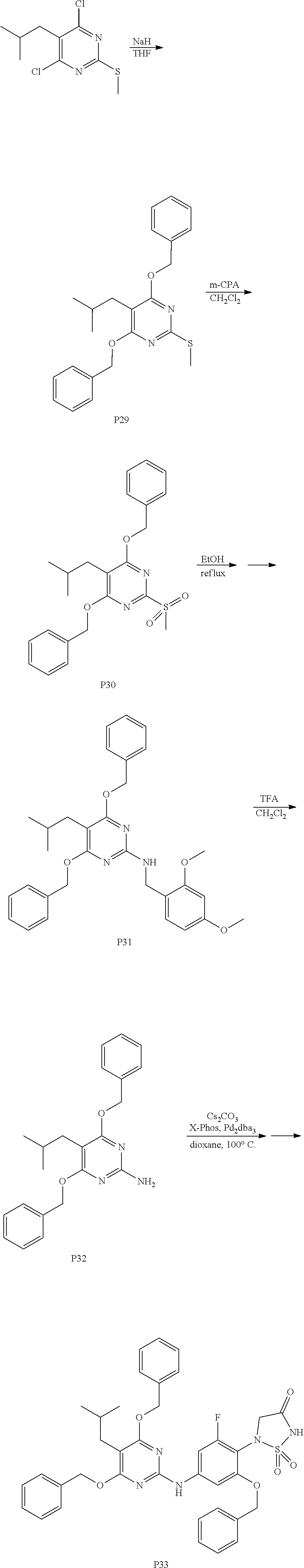

### Preparation 29. 4,6-Dibenzyloxy-5-isobutyl-2-methylsulfanyl-pyrimidine (P29)

To a stirred mixture of 4,6-dichloro-5-isobutyl-2-methylsulfanyl-pyrimidine (1.2 g, 4.7 mmol) in THF (5 mL) at 20° C. was added benzyl alcohol (1.2 g, 11 mmol) followed by NaH (60% in oil, 0.53 g, 13 mmol). The mixture was stirred at 50° C. until complete consumption of the starting material (TLC, 48 h). The solvent was evaporated in vacuum, DCM (10 mL) was then added, the mixture adsorbed onto silica and after column chromatography (n-hexane/DCM 70:30) the title compound P29 was obtained with yield 1.85 g (95%). ¹H NMR (400 MHz, DMSO-d₆), δ: 0.79 (s, 3H), 0.81 (s, 3H), 1.75-1.89 (m, 1H), 2.31 (d, 2H), 2.49 (s, 3H), 5.38 (s, 4H), 7.28-7.46 (m, 10H). LCMS (ESI⁺) m/z: 395 [M+H]⁺.

### Preparation 30. 4,6-Dibenzyloxy-5-isobutyl-2-methylsulfonyl-pyrimidine (P30)

1 g (2 mmol) of 4,6-dibenzyloxy-5-isobuthyl-2-methylsulfanyl-pyrimidine (P29) was initially charged in 35 ml of DCM, and 0.86 g (5 mmol) of 3-chloroperbenzoic acid was added at rt. After 10 h, the reaction was diluted with DCM and washed twice with 1M aq. sodium hydroxide solution, then with saturated aq. sodium sulfite solution and finally with water. The organic phase was dried over sodium sulfate, filtered and the solvent was removed under reduced pressure. The residue was purified by flash chromatography on silica gel eluting with DCM to give 0.4 g (47%) of title compound—P30. ¹H NMR (400 MHz, CDCl₃), δ: 0.89 (s, 3H), 0.90 (s, 3H), 1.92-2.10 (m, 1H), 2.52 (d, 2H), 3.24 (s, 3H), 5.49 (s, 4H), 7.32-7.46 (m, 10H). LCMS (ESI⁺) m/z: 426 [M+H]⁺.

### Preparation 31. 4,6-Dibenzyloxy-N-[(2,4-dimethoxyphenyl)methyl]-5-isobutyl-pyrimidin-2-amine (P31)

0.4 g (0.9 mmol) of 4,6-dibenzyloxy-5-isobutyl-2-methylsulfonyl-pyrimidine (P30) and 0.23 g (1.4 mmol) 2,4-dimethoxybenzyl amine were dissolved in 10 ml of ethanol and refluxed for 5 days, the reaction mixture was concentrated under reduced pressure. The residue was purified by flash chromatography on silica gel eluting with DCM/ethyl acetate (7:1) to give 0.3 g (62%) of the target product P31. ¹H NMR (400 MHz, DMSO-d₆), δ: 0.76 (s, 3H), 0.78 (s, 3H), 1.66-1.84 (m, 1H), 2.2 (d, 2H), 3.72 (s, 3H), 3.78 (s, 3H), 4.34 (s, 2H), 5.29 (s, 2H), 6.40-6.44 (m, 1H), 6.53 (d, 1H), 7.05-7.12 (m, 3H) 7.25-7.47 (m, 10H). LCMS (ESI⁺) m/z: 514 [M+H]⁺.

### Preparation 32. 4,6-Dibenzyloxy-5-isobutyl-pyrimidin-2-amine (P32)

0.3 g (2.4 mmol) of 4,6-dibenzyloxy-N-[(2,4-dimethoxyphenyl)methyl]-5-isobutyl-pyrimidin-2-amine (P31) was dissolved in 10 ml of DCM and 2.8 g (24.8 mmol) of TFA was added to the solution. The reaction mixture was stirred at rt for 3 h. After completing of the reaction 25 g (0.18 mol) of potassium carbonate and 2 ml of water was added to reaction mixture, the organic phase was separated and concentrated under reduced pressure. The residue was used in next step without purification. Yield 0.2 g (94%) of title compound P32. ¹H NMR (400 MHz, DMSO-d₆), δ: 0.78 (s, 3H), 0.8 (s, 3H), 1.72-1.82 (m, 1H), 2.23 (d, 2H), 5.30 (s, 4H), 6.35 (br. s, 2H), 7.28-7.42 (m, 5H). LCMS (ESI⁺) m/z: 364 [M+H]⁺.

### Preparation 33. 5-[2-Benzyloxy-4-[(4,6-dibenzyloxy-5-isobutyl-pyrimidin-2-yl)amino]-6-fluoro-phenyl]-1,1-dioxo-1,2,5-thiadiazolidin-3-one (P33)

0.2 g (0.55 mmol) of 4,6-dibenzyloxy-5-isobutyl-pyrimidin-2-amine (P32), 0.2 g (0.45 mmol) of 5-[2-(benzyloxy)-4-bromo-6-fluorophenyl]-1,2,5-thiadiazolidin-3-one-1,1-dioxide, and 0.3 g (0.9 mmol) of Cs₂CO₃ were dissolved in 10 ml of dioxane and stirred in the steam of N₂ for 15 min. 0.026 g (0.045 mmol) of X-Phos and 0.041 g (0.045 mol) of Pd₂dba₃ were added to the reaction mixture and stirred at 100° C. in the steam of N₂ overnight. 15 mL of EtOAc was added to the reaction mixture after it was cooled and formed precipitate was removed by filtration and filtrate was evaporated to dryness. The residue was purified by column chromatography on silica gel eluting with ethyl acetate/methanol (4:1) to yield 0.16 g (50%) of the title compound P33. ¹H NMR (400 MHz, DMSO-d₆), δ: 0.81 (s, 3H), 0.83 (s, 3H), 1.79-1.92 (m, 1H), 2.32 (d, 2H), 3.93 (s, 2H), 5.1 (s, 2H), 5.47 (s, 4H), 7.24-7.45 (m, 17H), 7.55 (s, 1H), 9.58 (s, 1H). LCMS (ESI⁺) m/z: 698 [M+H]⁺.

### Synthesis of 5-[2-benzyloxy-4-[(4-benzyloxy-6-methyl-5-propyl-pyrimidin-2-yl)amino]-6-fluoro-phenyl]-1,1-dioxo-1,2,5-thiadiazolidin-3-one (P41)

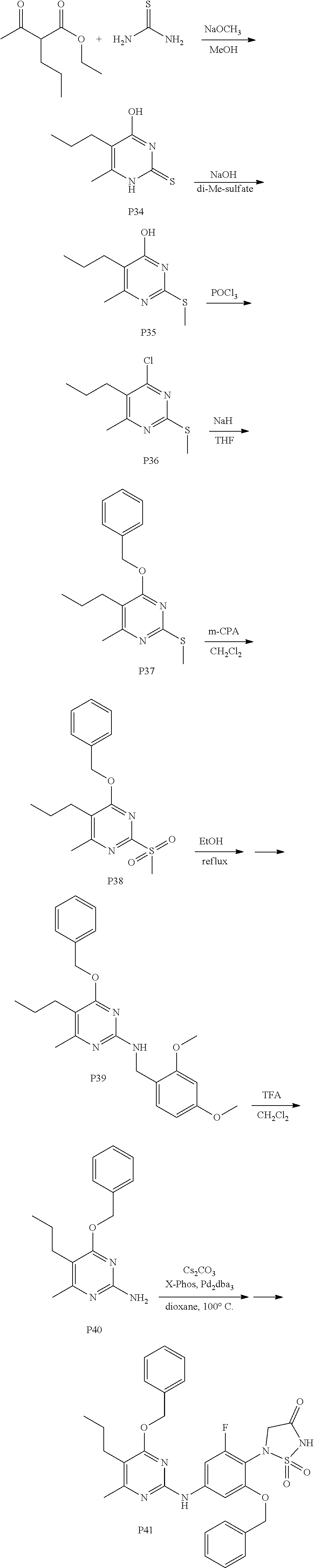

### Preparation 34. 4-Hydroxy-6-methyl-5-propyl-1H-pyrimidine-2-thione (P34)

To a solution of sodium methoxide (1.1 g, 0.02 mol) in 20 mL of absolute methanol thiourea (1.5 g, 0.02 mol) and ethyl 2-propylacetoacetate (3.44 g, 0.02 mol) were added. The mixture was boiled for 18 h with stirring. After cooling, the mixture was poured into ice water and acidified with conc. hydrochloric acid until acidic. The precipitate was filtered off, washed with ethanol, and dried to give 3.2 g (88%) of P34. ¹H NMR (400 MHz, DMSO-d₆), δ: 0.85 (t, 3H), 1.35 (m, 2H), 2.10 (s, 3H), 2.20 (t, 2H), 12.04 (s, 1H), 12.25 (s, 1H).

### Preparation 35. 6-Methyl-2-methylsulfanyl-5-propyl-pyrimidin-4-ol (P35)

2.4 g (0.019 mol) of dimethylsulphate was added slowly to a solution of 3.2 g (0.017 mol) of 4-hydroxy-6-methyl-5-propyl-1H-pyrimidine-2-thione (P34) in water (100 ml), containing 0.9 g (0.023 mol) of sodium hydroxide. The mixture was vigorously stirred at rt for 1 h and precipitated methylthio compound was collected. Yield of P35: 2.5 g (73%). ¹H NMR (400 MHz, DMSO-d₆), δ: 0.85 (m, 3H), 1.40 (m, 3H), 2.20 (s, 3H), 2.32 (m, 2H), 2.44 (s, 3H), 12.5 (br. s, 1H).

### Preparation 36. 4-Chloro-6-methyl-2-methylsulfanyl-5-propyl-pyrimidine (P36)

2.5 g (0.012 mol) of 6-methyl-2-methylsulfanyl-5-propyl-pyrimidin-4-ol (P35) and phosphorous oxychloride (20 mL) were combined and refluxed for 3 h. The reaction mixture was cooled to rt and poured onto crushed ice. The resultant aqueous mixture was extracted with ethyl acetate and the organic layer washed with sat. aq. sodium bicarbonate followed by a water wash, dried over magnesium sulfate, and dried in vacuo to give 4-chloro-5-propyl-6-methyl-2-(methylthio)pyrimidine P36 (2.67 g, 98%). ¹H NMR (400 MHz, DMSO-d₆), δ: 0.95 (t, 3H), 1.48-1.54 (m, 2H), 2.48 (br. s, 6H), 2.61-2.65 (m, 2H).

### Preparation 37. 4-Benzyloxy-6-methyl-2-methylsulfanyl-5-propyl-pyrimidine (P37)

To a stirred suspension of sodium hydride (0.6 g, 60% dispersion in mineral oil) in THF (10 ml) at 0° C. was added benzyl alcohol (1.43 g, 0.013 mol). The mixture was stirred for 15 min then a solution of 2.6 g (0.012 mol) 4-chloro-6-methyl-2-methylsulfanyl-5-propyl-pyrimidine (P36) in THF (5 ml) was added dropwise. The reaction was allowed to warm to rt and stirred for 8 h. Solvent was evaporated under reduced pressure and the remaining residue dissolved in DCM (50 ml) and washed with water (2×50 ml). Organics were separated, dried over sodium sulfate and the solvent was removed under reduced pressure and the residue was purified by flash chromatography on silica gel eluting with hexane/DCM (2:1) to give 3.3 g (98%) of P37. ¹H NMR (400 MHz, DMSO-d₆), δ: 0.88 (t, 3H), 1.44-1.49 (m, 2H), 2.34 (s, 3H), 2.46 (s, 3H), 2.47-2.49 (m, 2H), 2.41 (s, 2H), 7.30-7.45 (m, 5H). LCMS (ESI⁺) m/z: 289 [M+H]⁺.

### Preparation 38. 4-Benzyloxy-6-methyl-2-methylsulfonyl-5-propyl-pyrimidine (P38)

3.3 g (0.011 mol) of 4-benzyloxy-6-methyl-2-methylsulfanyl-5-propyl-pyrimidine (P37) was initially charged in 25 ml of DCM, and 4.9 g (0.28 mol) of 3-chloroperbenzoic acid was added at rt. After 2 h, the reaction was diluted with DCM and washed twice with 1M aq. sodium hydroxide solution, then with sat. aq. sodium sulfite solution and finally with water. The organic phase was dried over sodium sulfate, filtered and the solvent was removed under reduced pressure. The residue was purified by flash chromatography on silica gel eluting with DCM to yield 1.8 g (53%) of P38. ¹H NMR (400 MHz, DMSO-d₆), δ: 0.90 (t, 3H), 1.48-1.54 (m, 2H), 2.51 (s, 3H), 2.62 (t, 2H), 3.34 (s, 3H), 5.5 (s, 2H), 7.32-7.49 (m, 5H). LCMS (ESI⁺) m/z: 321 [M+H]⁺.

### Preparation 39. 4-Benzyloxy-N-[(2,4-dimethoxyphenyl)methyl]-6-methyl-5-propyl-pyrimidin-2-amine (P39)

1.5 g (4.6 mmol) of 4-benzyloxy-6-methyl-2-methylsulfonyl-5-propyl-pyrimidine (P38) and 1.17 g (7 mmol) of 2,4-dimethoxybenzyl amine were dissolved in 10 ml of ethanol and refluxed for 72 h. Then the reaction mixture was concentrated under reduced pressure. Residue was purified by flash chromatography on silica gel eluting with DCM/ethyl acetate (7:1) to give 0.5 g (26%) of P39. ¹H NMR (400 MHz, DMSO-d₆), δ: 0.85 (t, 3H), 1.36-1.42 (m, 2H), 2.18 (s, 3H), 2.33-2.38 (m, 2H), 3.72 (s, 3H), 3.78 (s, 3H), 3.34 (d, 2H), 5.29 (s, 2H), 6.40-6.42 (m, 1H), 6.52 (d, 1H), 7.07 (d, 1H), 7.25-7.45 (m, 5H). LCMS (ESI⁺) m/z: 408 [M+H]⁺.

### Preparation 40. 4-Benzyloxy-6-methyl-5-propyl-pyrimidin-2-amine (P40)

0.3 g (1.2 mmol) of 4-benzyloxy-N-[(2,4-dimethoxyphenyl)methyl]-6-methyl-5-propyl-pyrimidin-2-amine was dissolved in 10 mL of DCM and 1.4 g (12 mmol) of TFA was added to the solution. The reaction mixture was stirred at rt for 1 h. After completing of the reaction 1.6 g (12 mmol) of potassium carbonate and 0.5 ml of water were added to the reaction mixture, organic phase was separated and concentrated under reduced pressure. The residue was used in next step without purification. Yield of P41: 0.28 g (88%). ¹H NMR (400 MHz, DMSO-d₆), δ: 0.85 (t, 3H), 1.38-1.44 (m, 2H), 2.18 (s, 3H), 2.36-2.40 (m, 2H), 5.31 (s, 2H), 6.31 (br. s, 2H), 7.31-7.42 (m, 5H). LCMS (ESI⁺) m/z: 258 [M+H]⁺.

### Preparation 41. 5-[2-Benzyloxy-4-[(4-benzyloxy-6-methyl-5-propyl-pyrimidin-2-yl)amino]-6-fluoro-phenyl]-1,1-dioxo-1,2,5-thiadiazolidin-3-one (P41)

0.28 g (1.1 mmol) of 4-benzyloxy-6-methyl-5-propyl-pyrimidin-2-amine (P40), 0.44 g (0.9 mmol) of 5-[2-(benzyloxy)-4-bromo-6-fluorophenyl]-1,2,5-thiadiazolidin-3-one-1,1-dioxide, and 0.63 g (1.9 mmol) Cs₂CO₃ were dissolved in 10 ml dioxane and stirred in the steam of N₂ for 15 min. X-Phos (0.058 g, 0.09 mmol) and Pd₂dba₃ (0.088 g, 0.09 mol) were added to the reaction mixture and stirred at 100° C. in the steam of N₂ overnight. 15 ml of EtOAc was added to the reaction mixture after it was cooled, and the precipitate was filtered off and filtrate was evaporated to dryness. Residue was purified by column chromatography on silica gel eluting with ethyl acetate/methanol (4:1) to give 0.28 g (48%) of P41. ¹H NMR (400 MHz, DMSO-d₆), δ: 0.89 (t, 3H), 1.46-1.49 (m, 2H), 2.34 (s, 3H), 2.44-2.49 (m, 2H), 4.32 (s, 2H), 5.13 (s, 2H), 5.48 (s, 2H), 7.31-7.45 (m, 11H), 7.62 (s, 1H), 9.7 (s, 1H). LCMS (ESI⁺) m/z: 592 [M+H]⁺.

### Synthesis of 5-[2-benzyloxy-4-[(4-benzyloxy-5,6,7,8-tetrahydroquinazolin-2-yl)amino]-6-fluoro-phenyl]-1,1-dioxo-1,2,5-thiadiazolidin-3-one (P49)

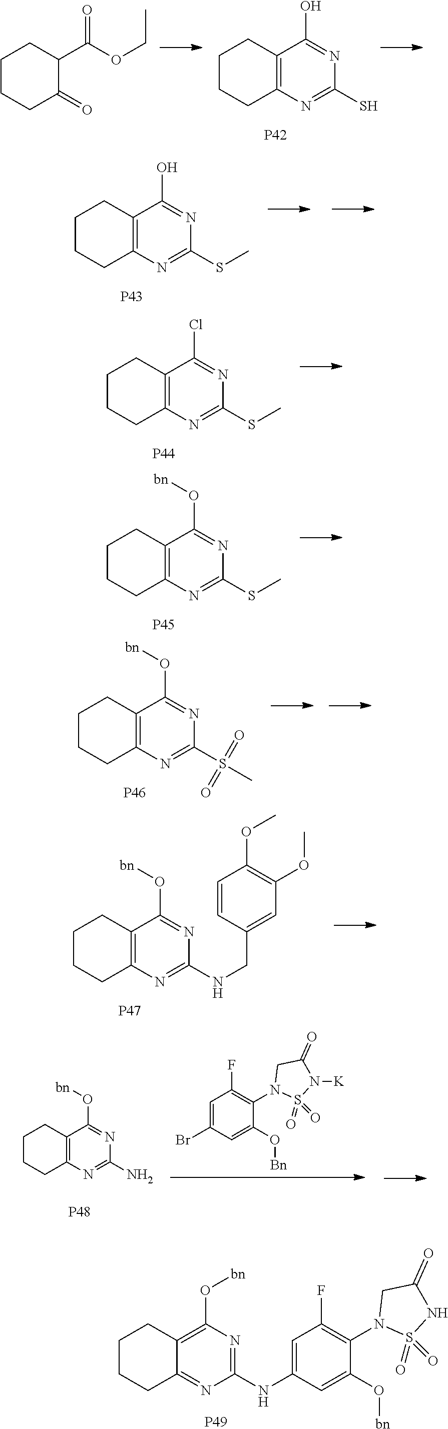

### Preparation 42. 2-Sulfanyl-5,6,7,8-tetrahydroquinazolin-4-ol (P42)

Ethyl 2-oxocyclohexanecarboxylate (8.5 g, 50 mmol, 1 eq) and thiourea (7.6 g, 100 mmol, 2 eq) were added at rt to a stirred solution of NaOMe in MeOH, which was obtained by adding sodium (3 g, 125 mmol, 2.5 eq) to 200 mL of MeOH. Resulting solution was stirred at reflux for 12 h, then all volatiles were removed under reduced pressure, and the residue was dissolved in H₂O (150 mL). Glacial AcOH was added dropwise to make the solution acidic while white precipitate was formed. It was collected and washed successively with sat. aq. NaHCO₃ solution (100 mL) and H₂O (100 mL) to give 2-sulfanyl-5,6,7,8-tetrahydroquinazolin-4-ol (P42, 7.55 g, 83%). LCMS (ESI⁺) m/z: 183 [M+H]⁺. ¹H NMR (400 MHz, DMSO-d₆), δ: 12.30 (s, 1H), 12.11 (s, 1H), 2.36 (t, J=5.5 Hz, 2H), 2.16 (t, J=5.5 Hz, 2H), 1.76-1.45 (m, 4H).

### Preparation 43. 2-Methylsulfanyl-5,6,7,8-tetrahydroquinazolin-4-ol (P43)

2-Sulfanyl-5,6,7,8-tetrahydroquinazolin-4-ol (P42, 7.5 g, 41 mmol, 1 eq) was dissolved in a NaOH solution, which was obtained by dissolving of solid NaOH (1.65 g, 41 mmol, 1 eq) in 50 mL of H₂O. The mixture was then treated with Mel (5.85 g, 41 mmol, 1 eq), and the resulting reaction mixture was allowed to stir for 16 h at rt. The solution was then acidified with glacial acetic acid until the white precipitate was formed. It was collected by suction filtration, and the solid was washed several times with cold water and dried to afford 2-methylsulfanyl-5,6,7,8-tetrahydroquinazolin-4-ol (P43, 5.24 g, 65%). ¹H NMR (400 MHz, DMSO-d₆), a: 12.22 (s, 1H), 2.44 (s, 3H), 2.47 (t, J=6.27 Hz, 2H), 2.27 (t, J=6.27 Hz, 2H), 1.66 (m, 4H). LCMS (ESI⁺) m/z: 197 [M+H]⁺.

### Preparation 44. 4-Chloro-2-methylsulfanyl-5,6,7,8-tetrahydroquinazoline (P44)

2-Methylsulfanyl-5,6,7,8-tetrahydroquinazolin-4-ol (P43, 5.24 g, 26.7 mmol, 1 eq) was refluxed for 5 h in POCl₃ (40 mL, 430 mmol, 16 eq). The solution was cooled to rt and then was poured over cracked ice and H₂O. The mixture was vigorously stirred at 0° C. for 1 h and extracted with EtOAc (3×50 mL). Combined organic phases were washed with sat. aq. NaHCO₃ solution (150 mL) and brine (100 mL), dried over Na₂SO₄ and concentrated under reduced pressure to provide 4-chloro-2-methylsulfanyl-5,6,7,8-tetrahydroquinazoline (P44, 5.7 g, 99%), which was used in the next step without further purification. ¹H NMR (400 MHz, CDCl₃), a: 2.81 (m, 2H), 2.71 (m, 2H), 2.56 (s, 3H), 1.85 (m, 4H). LCMS (ESI⁺) m/z: 215 [M+H]⁺.

### Preparation 45. 4-Benzyloxy-2-methylsulfanyl-5,6,7,8-tetrahydroquinazoline (P45)

A suspension of 4-chloro-2-methylsulfanyl-5,6,7,8-tetrahydroquinazoline (P44, 5.7 g, 26.5 mmol, 1 eq), BnOH (2.88 g, 27 mmol, 1 eq) and NaH (2.14 g, 54 mmol, 2 eq) was stirred in a dry THF (100 mL) at rt for 12 h. The excess of NaH was quenched by addition of saturated aqueous NH₄Cl solution (100 mL). The resulting solution was extracted with EtOAc (3×70 mL), combined organic phases were washed with brine (100 mL), dried over Na₂SO₄, and concentrated under reduced pressure to provide 4-benzyloxy-2-methylsulfanyl-5,6,7,8-tetrahydroquinazoline (P45, 7.6 g, 99%), which was used in the next step without further purification.

### Preparation 46. 4-benzyloxy-2-methylsulfonyl-5,6,7,8-tetrahydroquinazoline (P46)

MCPBA (70%, 11.5 g, 53 mmol, 2 eq) was added portionwise at 0° C. to a stirred solution of 4-benzyloxy-2-methylsulfanyl-5,6,7,8-tetrahydroquinazoline (P45, 7.6 g, 26.6 mmol, 1 eq) in DCM (200 mL). The reaction solution was stirred at rt for 16 h, formed precipitate was filtered off, and the filtrate was concentrated under reduced pressure. Residue was purified by column chromatography on SiO₂ (EtOAc/DCM=5/2) to give 4-benzyloxy-2-methylsulfonyl-5,6,7,8-tetrahydroquinazoline (P46, 8.4 g, 99%). ¹H NMR (400 MHz, CDCl₃), a: 7.54-7.29 (m, 5H), 5.52 (s, 2H), 3.28 (s, 3H), 2.89 (t, J=6.0 Hz, 2H), 2.68 (t, J=6.0 Hz, 2H), 2.00-1.71 (m, 4H). LCMS (ESI⁺) m/z: 319 [M+H]⁺.

### Preparation 47. 4-benzyloxy-N-[(3,4-dimethoxyphenyl)methyl]-5,6,7,8-tetrahydroquinazolin-2-amine (P47)

4-Benzyloxy-2-methylsulfonyl-5,6,7,8-tetrahydroquinazoline (P46, 3.18 g, 10 mmol) and (3,4-dimethoxyphenyl)methanamine (2.5 g, 15 mmol, 1.5 eq) in EtOH (50 mL) were stirred at reflux for 12 h. All volatiles were removed under reduced pressure, and the residue was purified by column chromatography on SiO₂ (Hex/EtOAc=5/2) to give 4-benzyloxy-N-[(3,4-dimethoxyphenyl)methyl]-5,6,7,8-tetrahydroquinazolin-2-amine (P47, 1 g, 25%). ¹H NMR (400 MHz, CDCl₃), δ: 7.45-7.30 (m, 5H), 7.18 (d, J=8.2 Hz, 1H), 6.46 (d, J=2.3 Hz, 1H), 6.38 (dd, J=8.2, 2.3 Hz, 1H), 5.42 (s, 2H), 4.53 (d, J=6.2 Hz, 2H), 3.83 (s, 3H), 3.80 (s, 3H), 2.60 (t, J=6.1 Hz, 2H), 2.48 (t, J=6.1 Hz, 2H), 1.84-1.67 (m, 4H). LCMS (ESI⁺) m/z: 406 [M+H]⁺.

### Preparation 48. 4-Benzyloxy-5,6,7,8-tetrahydroquinazolin-2-amine (P48)

To a stirred solution of 4-benzyloxy-N-[(3,4-dimethoxyphenyl)methyl]-5,6,7,8-tetrahydroquinazolin-2-amine (P47, 1 g, 2.46 mmol, 1 eq) in DCM (30 mL) TFA (3.8 mL, 50 mmol, 20 eq) was added. The reaction solution was stirred at rt for 3 h, all volatiles were removed under reduced pressure, and the residue was purified by column chromatography on SiO₂ (from EtOAc/DCM=5/2 to 100% of EtOAc) to obtain 4-benzyloxy-5,6,7,8-tetrahydroquinazolin-2-amine (P48, 285 mg, 45%). ¹H NMR (400 MHz, DMSO-d₆), δ: 7.36 (m, 5H), 6.17 (s, 2H), 5.31 (s, 2H), 3.32 (s, 2H), 2.46 (t, J=5.8 Hz, 2H), 2.37 (t, J=5.8 Hz, 2H), 1.67 (m, 4H). LCMS (ESI⁺) m/z: 256 [M+H]⁺.

### Preparation 49. 5-[2-benzyloxy-4-[(4-benzyloxy-5,6,7,8-tetrahydroquinazolin-2-yl)amino]-6-fluoro-phenyl]-1,1-dioxo-1,2,5-thiadiazolidin-3-one (P49)

A suspension of 4-benzyloxy-5,6,7,8-tetrahydroquinazolin-2-amine (P48, 275 mg, 1.08 mmol, 1 eq), [5-(2-benzyloxy-4-bromo-6-fluoro-phenyl)-1,1,3-trioxo-1,2,5-thiadiazolidin-2-yl]potassium (488 mg, 1.08 mmol, 1 eq), Cs₂CO₃ (880 mg, 2.7 mmol, 2.5 eq), XantPhos (60 mg, 0.1 mmol, 10% mol) and Pd₂dba₃ (50 mg, 0.05 mmol, 5% mol) was stirred under N₂ at 75° C. in dioxane (15 mL) for 12 h. A resulting solution was cooled to rt, acidified with 10% aqueous H₃PO₄ and extracted with EtOAc (3×10 mL). The combined organic phases were washed with brine, dried with Na₂SO₄ and then all volatiles were evaporated under reduced pressure. Residue was purified by column chromatography on SiO₂ (from EtOAc/MeOH=10/1 to EtOAc/MeOH=5/1) to obtain 5-[2-benzyloxy-4-[(4-benzyloxy-5,6,7,8-tetrahydroquinazolin-2-yl)amino]-6-fluoro-phenyl]-1,1-dioxo-1,2,5-thiadiazolidin-3-one (P49, 680 mg, 87%). LCMS (ESI⁺) m/z: 591 [M+H]⁺.

### Examples of the Final Compound

In the Table 3 presented certain non-limiting examples of the compound of Formula (I).

TABLE 3
Selected examples of the compound of Formula (I)

| | | | [MH]⁺ | [MH]⁺ |
|---|---|---|---|---|
| # | Structure | IUPAC Name | Calc. | Found |
| 1 | 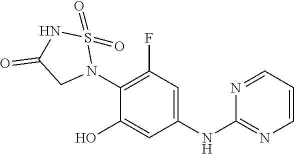 [SMILES: O=C1CN(c2c(O)cc(Nc3ncccn3)cc2F)S(=O)(=O)N1] | 5-[2-fluoro-6-hydroxy- 4-(pyrimidin-2- ylamino)phenyl]-1,1- dioxo-1,2,5- thiadiazolidin-3-one | 340.052 | 340 |
| 2 | 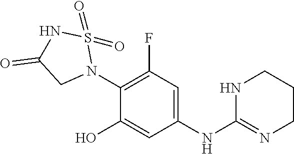 [SMILES: O=C1CN(c2c(O)cc(NC3=NCCCN3)cc2F)S(=O)(=O)N1] | 5-[2-fluoro-6-hydroxy- 4-(1,4,5,6- tetrahydropyrimidin-2- ylamino)phenyl]-1,1- dioxo-1,2,5- thiadiazolidin-3-one | 344.083 | 344 |
| 3 | 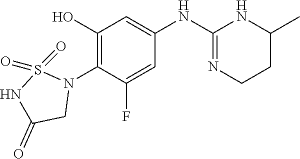 [SMILES: CC1CCN=C(Nc2cc(O)c(N3CC(=O)NS3(=O)=O)c(F)c2)N1] | 5-[2-fluoro-6-hydroxy- 4-[(6-methyl-1,4,5,6- tetrahydropyrimidin-2- yl)amino]phenyl]-1,1- dioxo-1,2,5- thiadiazolidin-3-one | 358.099 | 358 |
| 4 | 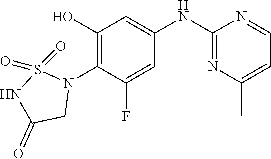 [SMILES: Cc1ccnc(Nc2cc(O)c(N3CC(=O)NS3(=O)=O)c(F)c2)n1] | 5-[2-fluoro-6-hydroxy- 4-[(4-methylpyrimidin- 2-yl)amino]phenyl]-1,1- dioxo-1,2,5- thiadiazolidin-3-one | 354.067 | 354 |
| 5 | 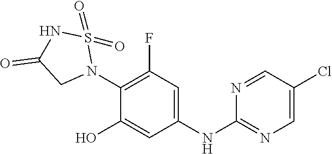 [SMILES: O=C1CN(c2c(O)cc(Nc3ncc(Cl)cn3)cc2F)S(=O)(=O)N1] | 5-[4-[(5- chloropyrimidin-2- yl)amino]-2-fluoro-6- hydroxy-phenyl]-1,1- dioxo-1,2,5- thiadiazolidin-3-one | 374.013 | 374 |
| 6 | 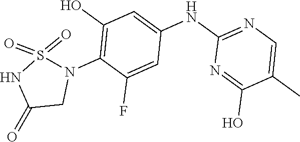 [SMILES: Cc1cnc(Nc2cc(O)c(N3CC(=O)NS3(=O)=O)c(F)c2)nc1O] | 5-[2-fluoro-6-hydroxy- 4-[(4-hydroxy-5-methyl- pyrimidin-2- yl)amino]phenyl]-1,1- dioxo-1,2,5- thiadiazolidin-3-one | 370.062 | 370 |
| 7 | 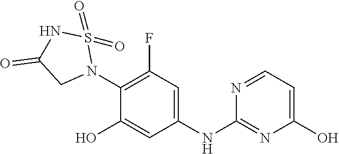 [SMILES: O=C1CN(c2c(O)cc(Nc3nccc(O)n3)cc2F)S(=O)(=O)N1] | 5-[2-fluoro-6-hydroxy- 4-[(4-hydroxypyrimidin- 2-yl)amino]phenyl]-1,1- dioxo-1,2,5- thiadiazolidin-3-one | 356.046 | 356 |
| 8 | 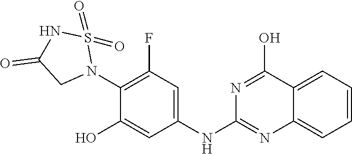 [SMILES: O=C1CN(c2c(O)cc(Nc3nc(O)c4ccccc4n3)cc2F)S(=O)(=O)N1] | 5-[2-fluoro-6-hydroxy- 4-[(4- hydroxyquinazolin-2- yl)amino]phenyl]-1,1- dioxo-1,2,5- thiadiazolidin-3-one | 406.062 | 406 |
| 9 | 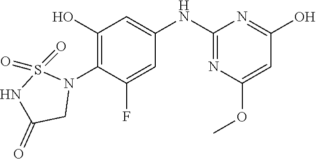 [SMILES: COc1cc(O)nc(Nc2cc(O)c(N3CC(=O)NS3(=O)=O)c(F)c2)n1] | 5-[2-fluoro-6-hydroxy- 4-[(4-hydroxy-6- methoxy-pyrimidin-2- yl)amino]phenyl]-1,1- dioxo-1,2,5- thiadiazolidin-3-one | 386.057 | 386 |
| 10 | 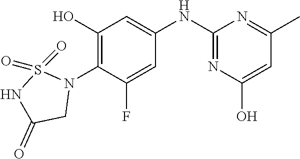 [SMILES: Cc1cc(O)nc(Nc2cc(O)c(N3CC(=O)NS3(=O)=O)c(F)c2)n1] | 5-[2-fluoro-6-hydroxy- 4-[(4-hydroxy-6-methyl- pyrimidin-2- yl)amino]phenyl]-1,1- dioxo-1,2,5- thiadiazolidin-3-one | 370.062 | 370 |
| 11 | 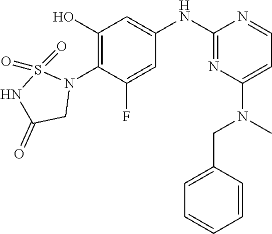 [SMILES: CN(Cc1ccccc1)c1ccnc(Nc2cc(O)c(N3CC(=O)NS3(=O)=O)c(F)c2)n1] | 5-[4-[[4- [benzyl(methyl)amino] pyrimidin-2-yl]amino]-2- fluoro-6-hydroxy- phenyl]-1,1-dioxo-1,2,5- thiadiazolidin-3-one | 459.125 | 459 |
| 12 | 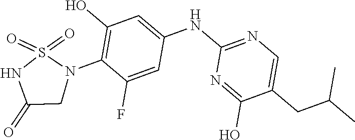 [SMILES: CC(C)Cc1cnc(Nc2cc(O)c(N3CC(=O)NS3(=O)=O)c(F)c2)nc1O] | 5-[2-fluoro-6-hydroxy- 4-[(4-hydroxy-5- isobutyl-pyrimidin-2- yl)amino]phenyl]-1,1- dioxo-1,2,5- thiadiazolidin-3-one | 412.109 | 412 |
| 13 | 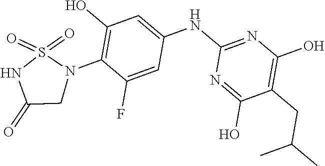 [SMILES: CC(C)Cc1c(O)nc(Nc2cc(O)c(N3CC(=O)NS3(=O)=O)c(F)c2)nc1O] | 5-[4-[(4,6-dihydroxy-5- isobutyl-pyrimidin-2- yl)amino]-2-fluoro-6- hydroxy-phenyl]-1,1- dioxo-1,2,5- thiadiazolidin-3-one | 428.104 | 428 |
| 14 | 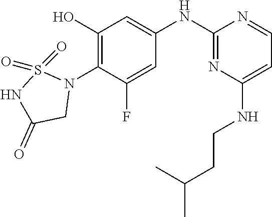 [SMILES: CC(C)CCNc1ccnc(Nc2cc(O)c(N3CC(=O)NS3(=O)=O)c(F)c2)n1] | 5-[2-fluoro-6-hydroxy- 4-[[4- (isopentylamino) pyrimidin- 2-yl]amino]phenyl]- 1,1-dioxo-1,2,5- thiadiazolidin-3-one | 425.141 | 425 |
| 15 | 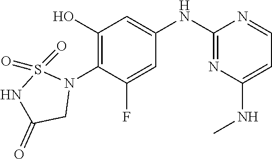 [SMILES: CNc1ccnc(Nc2cc(O)c(N3CC(=O)NS3(=O)=O)c(F)c2)n1] | 5-[2-fluoro-6-hydroxy- 4-[[4- (methylamino)pyrimidin- 2-yl]amino]phenyl]- 1,1-dioxo-1,2,5- thiadiazolidin-3-one | 369.078 | 369 |
| 16 | 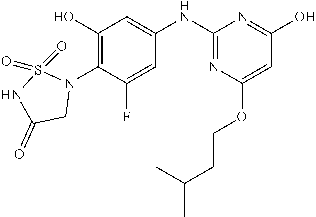 [SMILES: CC(C)CCOc1cc(O)nc(Nc2cc(O)c(N3CC(=O)NS3(=O)=O)c(F)c2)n1] | 5-[2-fluoro-6-hydroxy- 4-[(4-hydroxy-6- isopentyloxy-pyrimidin- 2-yl)amino]phenyl]-1,1- dioxo-1,2,5- thiadiazolidin-3-one | 442.12 | 442 |
| 17 | 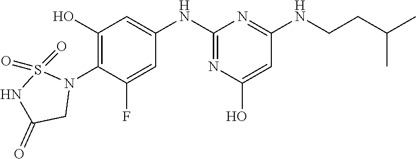 [SMILES: CC(C)CCNc1cc(O)nc(Nc2cc(O)c(N3CC(=O)NS3(=O)=O)c(F)c2)n1] | 5-[2-fluoro-6-hydroxy- 4-[[4-hydroxy-6- (isopentylamino) pyrimidin- 2-yl]amino]phenyl]- 1,1-dioxo-1,2,5- thiadiazolidin-3-one | 441.136 | 441 |
| 18 | 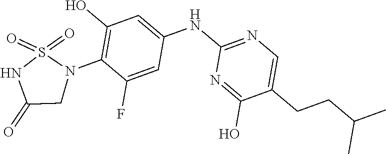 [SMILES: CC(C)CCc1cnc(Nc2cc(O)c(N3CC(=O)NS3(=O)=O)c(F)c2)nc1O] | 5-[2-fluoro-6-hydroxy- 4-[(4-hydroxy-5- isopentyl-pyrimidin-2- yl)amino]phenyl]-1,1- dioxo-1,2,5- thiadiazolidin-3-one | 426.125 | 426 |
| 19 | 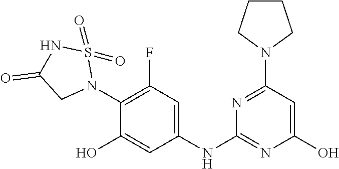 [SMILES: O=C1CN(c2c(O)cc(Nc3nc(O)cc(N4CCCC4)n3)cc2F)S(=O)(=O)N1] | 5-[2-fluoro-6-hydroxy- 4-[(4-hydroxy-6- pyrrolidin-1-yl- pyrimidin-2- yl)amino]phenyl]-1,1- dioxo-1,2,5- thiadiazolidin-3-one | 425.104 | 425 |
| 20 | 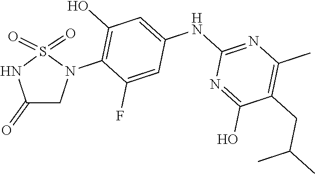 [SMILES: Cc1nc(Nc2cc(O)c(N3CC(=O)NS3(=O)=O)c(F)c2)nc(O)c1CC(C)C] | 5-[2-fluoro-6-hydroxy- 4-[(4-hydroxy-5- isobutyl-6-methyl- pyrimidin-2- yl)amino]phenyl]-1,1- dioxo-1,2,5- thiadiazolidin-3-one | 426.125 | 426 |
| 21 | 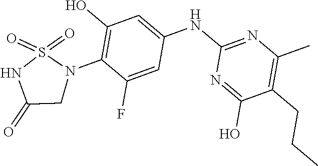 [SMILES: CCCc1c(C)nc(Nc2cc(O)c(N3CC(=O)NS3(=O)=O)c(F)c2)nc1O] | 5-[2-fluoro-6-hydroxy- 4-[(4-hydroxy-6-methyl- 5-propyl-pyrimidin-2- yl)amino]phenyl]-1,1- dioxo-1,2,5- thiadiazolidin-3-one | 412.109 | 412 |
| 22 | 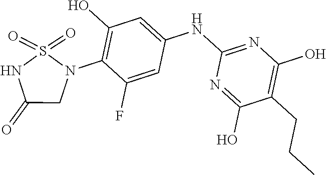 [SMILES: CCCc1c(O)nc(Nc2cc(O)c(N3CC(=O)NS3(=O)=O)c(F)c2)nc1O] | 5-[4-[(4,6-dihydroxy-5- propyl-pyrimidin-2- yl)amino]-2-fluoro-6- hydroxy-phenyl]-1,1- dioxo-1,2,5- thiadiazolidin-3-one | 414.088 | 414 |
| 23 | 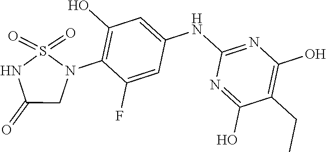 [SMILES: CCc1c(O)nc(Nc2cc(O)c(N3CC(=O)NS3(=O)=O)c(F)c2)nc1O] | 5-[4-[(5-ethyl-4,6- dihydroxy-pyrimidin-2- yl)amino]-2-fluoro-6- hydroxy-phenyl]-1,1- dioxo-1,2,5- thiadiazolidin-3-one | 400.073 | 400 |
| 24 | 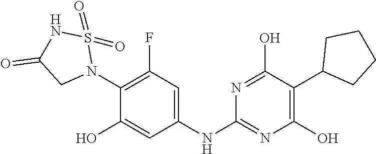 [SMILES: O=C1CN(c2c(O)cc(Nc3nc(O)c(C4CCCC4)c(O)n3)cc2F)S(=O)(=O)N1] | 5-[4-[(5-cyclopentyl- 4,6-dihydroxy- pyrimidin-2-yl)amino]- 2-fluoro-6-hydroxy- phenyl]-1,1-dioxo-1,2,5- thiadiazolidin-3-one | 440.104 | 440 |
| 25 | 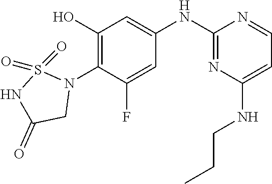 [SMILES: CCCNc1ccnc(Nc2cc(O)c(N3CC(=O)NS3(=O)=O)c(F)c2)n1] | 5-[2-fluoro-6-hydroxy- 4-[[4- (propylamino)pyrimidin- 2-yl]amino]phenyl]- 1,1-dioxo-1,2,5- thiadiazolidin-3-one | 397.109 | 397 |
| 26 | 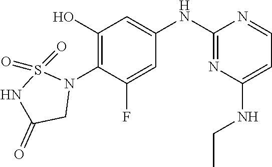 [SMILES: CCNc1ccnc(Nc2cc(O)c(N3CC(=O)NS3(=O)=O)c(F)c2)n1] | 5-[4-[[4- (ethylamino)pyrimidin- 2-yl]amino]-2-fluoro-6- hydroxy-phenyl]-1,1- dioxo-1,2,5- thiadiazolidin-3-one | 383.094 | 383 |
| 27 | 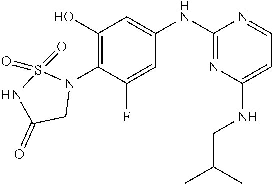 [SMILES: CC(C)CNc1ccnc(Nc2cc(O)c(N3CC(=O)NS3(=O)=O)c(F)c2)n1] | 5-[2-fluoro-6-hydroxy- 4-[[4- (isobutylamino) pyrimidin- 2-yl]amino]phenyl]- 1,1-dioxo-1,2,5- thiadiazolidin-3-one | 411.125 | 411 |
| 28 | 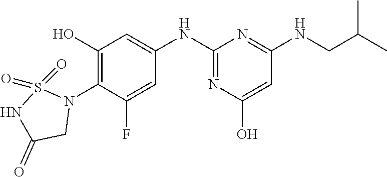 [SMILES: CC(C)CNc1cc(O)nc(Nc2cc(O)c(N3CC(=O)NS3(=O)=O)c(F)c2)n1] | 5-[2-fluoro-6-hydroxy- 4-[[4-hydroxy-6- (isobutylamino) pyrimidin- 2-yl]amino]phenyl]- 1,1-dioxo-1,2,5- thiadiazolidin-3-one | 427.12 | 427 |
| 29 | 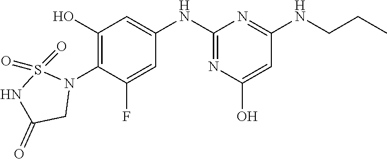 [SMILES: CCCNc1cc(O)nc(Nc2cc(O)c(N3CC(=O)NS3(=O)=O)c(F)c2)n1] | 5-[2-fluoro-6-hydroxy- 4-[[4-hydroxy-6- (propylamino)pyrimidin- 2-yl]amino]phenyl]- 1,1-dioxo-1,2,5- thiadiazolidin-3-one | 413.104 | 413 |
| 30 | 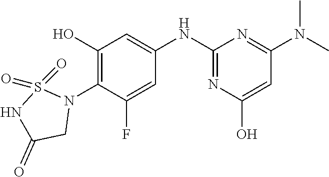 [SMILES: CN(C)c1cc(O)nc(Nc2cc(O)c(N3CC(=O)NS3(=O)=O)c(F)c2)n1] | 5-[4-[[4- (dimethylamino)-6- hydroxy-pyrimidin-2- yl]amino]-2-fluoro-6- hydroxy-phenyl]-1,1- dioxo-1,2,5- thiadiazolidin-3-one | 399.089 | 399 |
| 31 | 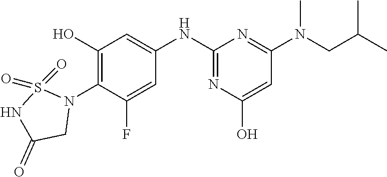 [SMILES: CC(C)CN(C)c1cc(O)nc(Nc2cc(O)c(N3CC(=O)NS3(=O)=O)c(F)c2)n1] | 5-[2-fluoro-6-hydroxy- 4-[[4-hydroxy-6- [isobutyl(methyl)amino] pyrimidin-2- yl]amino]phenyl]-1,1- dioxo-1,2,5- thiadiazolidin-3-one | 441.136 | 441 |
| 32 | 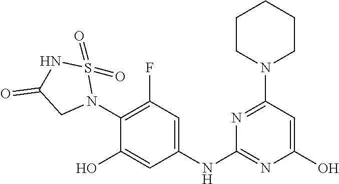 [SMILES: O=C1CN(c2c(O)cc(Nc3nc(O)cc(N4CCCCC4)n3)cc2F)S(=O)(=O)N1] | 5-[2-fluoro-6-hydroxy- 4-[[4-hydroxy-6-(1- piperidyl)pyrimidin-2- yl]amino]phenyl]-1,1- dioxo-1,2,5- thiadiazolidin-3-one | 439.12 | 439 |
| 33 | 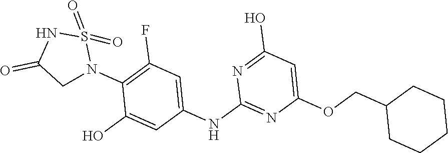 [SMILES: O=C1CN(c2c(O)cc(Nc3nc(O)cc(OCC4CCCCC4)n3)cc2F)S(=O)(=O)N1] | 5-[4-[[4- (cyclohexylmethoxy)-6- hydroxy-pyrimidin-2- yl]amino]-2-fluoro-6- hydroxy-phenyl]-1,1- dioxo-1,2,5- thiadiazolidin-3-one | 468.135 | 468 |
| 34 | 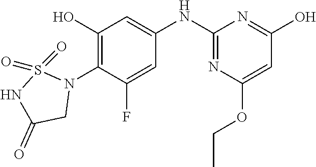 [SMILES: CCOc1cc(O)nc(Nc2cc(O)c(N3CC(=O)NS3(=O)=O)c(F)c2)n1] | 5-[4-[(4-ethoxy-6- hydroxy-pyrimidin-2- yl)amino]-2-fluoro-6- hydroxy-phenyl]-1,1- dioxo-1,2,5- thiadiazolidin-3-one | 400.073 | 400 |
| 35 | 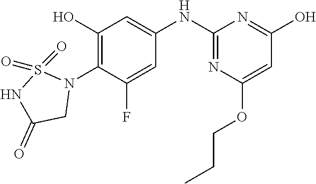 [SMILES: CCCOc1cc(O)nc(Nc2cc(O)c(N3CC(=O)NS3(=O)=O)c(F)c2)n1] | 5-[2-fluoro-6-hydroxy- 4-[(4-hydroxy-6- propoxy-pyrimidin-2- yl)amino]phenyl]-1,1- dioxo-1,2,5- thiadiazolidin-3-one | 414.088 | 414 |
| 36 | 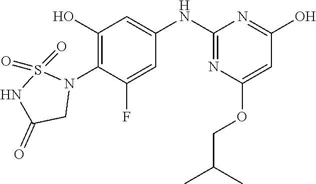 [SMILES: CC(C)COc1cc(O)nc(Nc2cc(O)c(N3CC(=O)NS3(=O)=O)c(F)c2)n1] | 5-[2-fluoro-6-hydroxy- 4-[(4-hydroxy-6- isobutoxy-pyrimidin-2- yl)amino]phenyl]-1,1- dioxo-1,2,5- thiadiazolidin-3-one | 428.104 | 428 |
| 37 | 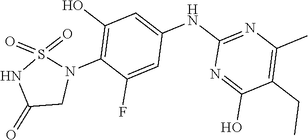 [SMILES: CCc1c(C)nc(Nc2cc(O)c(N3CC(=O)NS3(=O)=O)c(F)c2)nc1O] | 5-[4-[(5-ethyl-4- hydroxy-6-methyl- pyrimidin-2-yl)amino]- 2-fluoro-6-hydroxy- phenyl]-1,1-dioxo-1,2,5- thiadiazolidin-3-one | 398.093 | 398 |
| 38 | 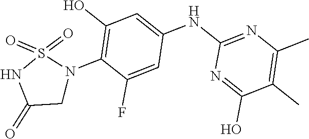 [SMILES: Cc1nc(Nc2cc(O)c(N3CC(=O)NS3(=O)=O)c(F)c2)nc(O)c1C] | 5-[2-fluoro-6-hydroxy- 4-[(4-hydroxy-5,6- dimethyl-pyrimidin-2- yl)amino]phenyl]-1,1- dioxo-1,2,5- thiadiazolidin-3-one | 384.078 | 384 |
| 39 | 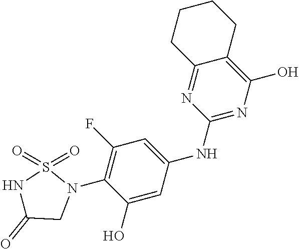 [SMILES: O=C1CN(c2c(O)cc(Nc3nc(O)c4c(n3)CCCC4)cc2F)S(=O)(=O)N1] | 5-[2-fluoro-6-hydroxy- 4-[(4-hydroxy-5,6,7,8- tetrahydroquinazolin-2- yl)amino]phenyl]-1,1- dioxo-1,2,5- thiadiazolidin-3-one | 410.093 | 411 |
| 40 | 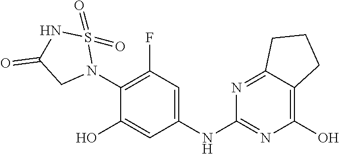 [SMILES: O=C1CN(c2c(O)cc(Nc3nc(O)c4c(n3)CCC4)cc2F)S(=O)(=O)N1] | 5-[2-fluoro-6-hydroxy- 4-[(4-hydroxy-6,7- dihydro-5H- cyclopenta[d]pyrimidin- 2-yl)amino]phenyl]-1,1- dioxo-1,2,5- thiadiazolidin-3-one | 396.078 | 396 |
| 41 | 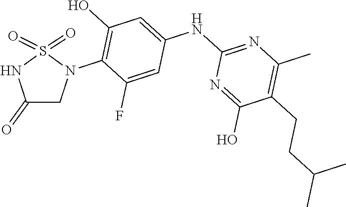 [SMILES: Cc1nc(Nc2cc(O)c(N3CC(=O)NS3(=O)=O)c(F)c2)nc(O)c1CCC(C)C] | 5-[2-fluoro-6-hydroxy- 4-[(4-hydroxy-5- isopentyl-6-methyl- pyrimidin-2- yl)amino]phenyl]-1,1- dioxo-1,2,5- thiadiazolidin-3-one | 440.14 | 440 |
| 42 | 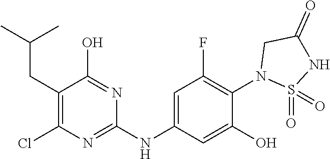 [SMILES: CC(C)Cc1c(O)nc(Nc2cc(O)c(N3CC(=O)NS3(=O)=O)c(F)c2)nc1Cl] | 5-[4-[(4-chloro-6- hydroxy-5-isobutyl- pyrimidin-2-yl)amino]- 2-fluoro-6-hydroxy- phenyl]-1,1-dioxo-1,2,5- thiadiazolidin-3-one | 446.07 | 446 |

### Synthesis of the Representative Examples of the Compound

### Example 1. 5-[2-Fluoro-6-hydroxy-4-(1,4,5,6-tetrahydropyrimidin-2-ylamino)phenyl]-1,1-dioxo-1,2,5-thiadiazolidin-3-one (Compound 2)

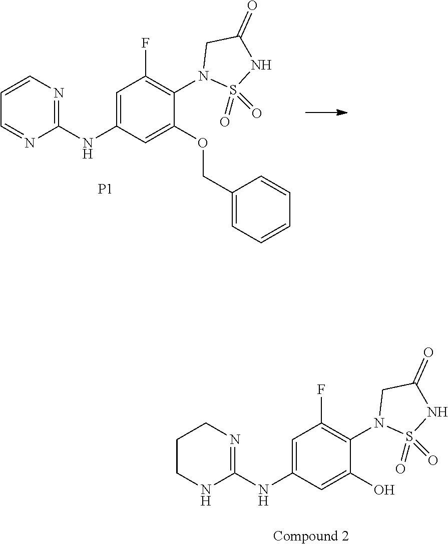 [SMILES: O=C1CN(c2c(F)cc(Nc3ncccn3)cc2OCc2ccccc2)S(=O)(=O)N1 ; "P1" ; O=C1CN(c2c(O)cc(NC3=NCCCN3)cc2F)S(=O)(=O)N1 ; "Compound 2"]

To a solution of 5-[2-benzyloxy-6-fluoro-4-(pyrimidin-2-ylamino)phenyl]-1,1-dioxo-1,2,5-thiadiazolidin-3-one (P1, 100 mg) and aq. conc. HCl (20 μL) in MeOH (15 mL) 10% wet Pd/C (20 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 2 (12 mg, 15%). ¹H NMR (400 MHz, DMSO-d₆), δ: 9.78 (s, 1H), 9.54 (s, 1H), 8.28 (s, 2H), 6.58 (dd, J=11.3, 2.5 Hz, 1H), 6.52 (m, 1H), 3.95 (s, 2H), 3.29 (s, 4H), 1.87 (t, J=5.8 Hz, 2H). LCMS (ESI⁺) m/z: 344 [M+H]⁺.

### Example 2. 5-[2-Fluoro-6-hydroxy-4-[(6-methyl-1,4,5,6-tetrahydropyrimidin-2-yl)amino]phenyl]-1,1-dioxo-1,2,5-thiadiazolidin-3-one (Compound 3)

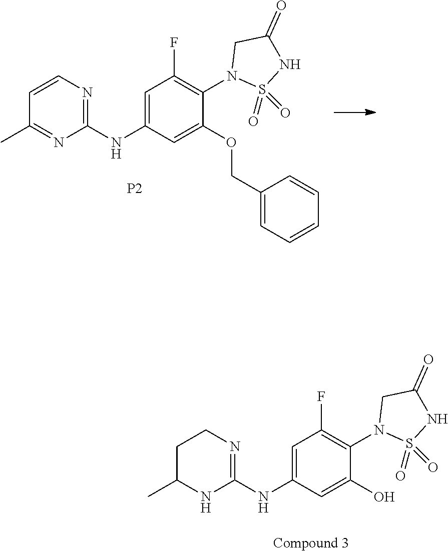

To a solution of 5-[2-benzyloxy-6-fluoro-4-[(4-methylpyrimidin-2-yl)amino]phenyl]-1,1-dioxo-1,2,5-thiadiazolidin-3-one (P2, 140 mg) and aq. conc. HCl (40 μL) in MeOH (25 mL) 10% wet Pd/C (30 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 3 (6 mg, 6%). LCMS (ESI⁺) m/z: 358 [M+H]⁺.

### Example 3. 5-[2-Fluoro-6-hydroxy-4-[(6-methyl-1H-pyrimidin-2-yl)amino]phenyl]-1,1-dioxo-1,2,5-thiadiazolidin-3-one (Compound 4)

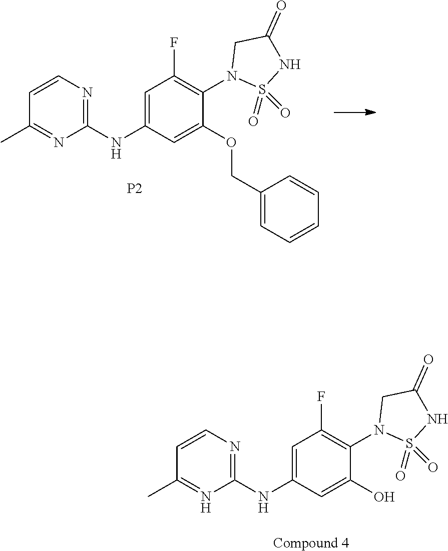

To a solution of 5-[2-benzyloxy-6-fluoro-4-[(4-methylpyrimidin-2-yl)amino]phenyl]-1,1-dioxo-1,2,5-thiadiazolidin-3-one (P2, 185 mg) in MeOH (25 mL) 10% wet Pd/C (20 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 4 (16 mg, 11%). ¹H NMR (400 MHz, DMSO-d₆), δ: 10.14 (s, 1H), 9.78 (s, 1H), 8.37 (d, J=5.0 Hz, 1H), 7.29 (dd, J=13.0, 2.3 Hz, 1H), 7.26 (dd, J=2.4, 1.3 Hz, 1H), 6.80 (d, J=5.0 Hz, 1H), 4.31 (s, 2H), 2.38 (s, 3H). LCMS (ESI⁺) m/z: 354 [M+H]⁺.

### Example 4. 5-[4-[(5-Chloropyrimidin-2-yl)amino]-2-fluoro-6-hydroxy-phenyl]-1,1-dioxo-1,2,5-thiadiazolidin-3-one (Compound 5)

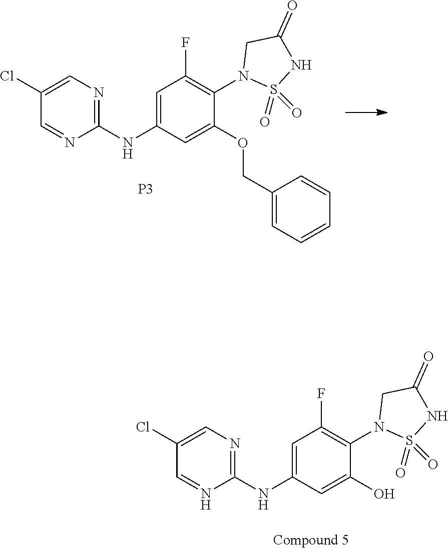

To a solution of 5-[2-benzyloxy-4-[(5-chloropyrimidin-2-yl)amino]-6-fluoro-phenyl]-1,1-dioxo-1,2,5-thiadiazolidin-3-one (P3, 170 mg) in MeOH (25 mL) 10% wet Pd/C (30 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 5 (17 mg, 12%). ¹H NMR (400 MHz, DMSO-d₆), δ: 10.30 (s, 1H), 10.09 (s, 1H), 8.61 (s, 2H), 7.24 (dd, J=12.8, 2.4 Hz, 1H), 7.17 (m, 1H), 4.34 (s, 2H). LCMS (ESI⁺) m/z: 374 [M+H]⁺.

### Example 5. 5-[2-Fluoro-6-hydroxy-4-[(4-hydroxy-5-methyl-pyrimidin-2-yl)amino]phenyl]-1,1-dioxo-1,2,5-thiadiazolidin-3-one (Compound 6)

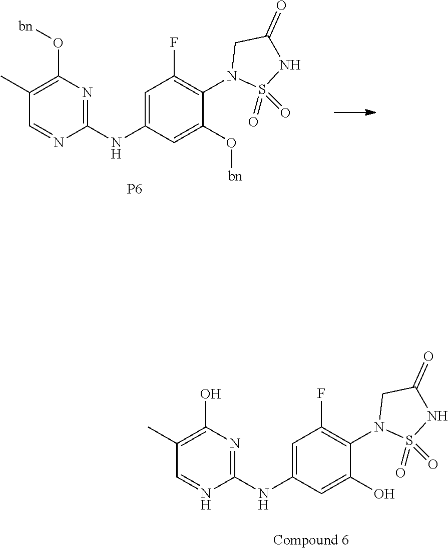

To a solution of 5-[2-benzyloxy-4-[(4-benzyloxy-5-methyl-pyrimidin-2-yl)amino]-6-fluoro-phenyl]-1,1-dioxo-1,2,5-thiadiazolidin-3-one (P6, 300 mg) in MeOH (20 mL) 10% wet Pd/C (50 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 6 (6 mg, 3%). ¹H NMR (400 MHz, DMSO-d₆), δ: 10.33 (s, 1H), 9.07 (s, 1H), 7.67 (s, 1H), 7.13 (dd, J=12.3, 2.4 Hz, 1H), 6.94 (m, 1H), 4.31 (s, 2H), 1.86 (s, 3H). LCMS (ESI⁺) m/z: 370 [M+H]⁺.

### Example 6. 5-[2-Fluoro-6-hydroxy-4-[(4-hydroxypyrimidin-2-yl)amino]phenyl]-1,1-dioxo-1,2,5-thiadiazolidin-3-one (Compound 7)

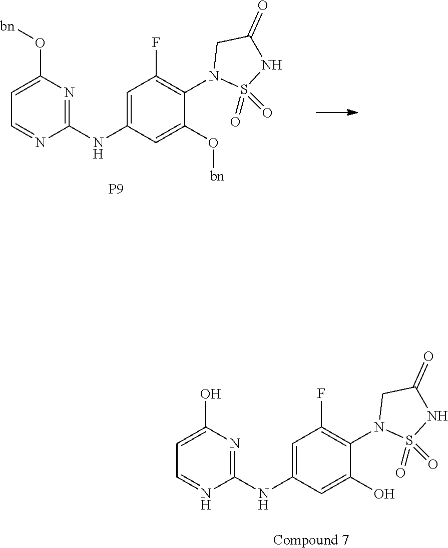

To a solution of 5-[2-benzyloxy-4-[(4-benzyloxypyrimidin-2-yl)amino]-6-fluoro-phenyl]-1,1-dioxo-1,2,5-thiadiazolidin-3-one (P9, 100 mg) in MeOH (20 mL) 10% wet Pd/C (20 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 7 (13 mg, 19%). ¹H NMR (400 MHz, DMSO-d₆), δ: 10.12 (s, 1H), 9.20 (s, 1H), 7.85 (s, 1H), 7.19 (d, J=12.7 Hz, 1H), 6.96 (s, 1H), 5.92 (d, J=7.0 Hz, 1H), 4.22 (s, 2H). LCMS (ESI⁺) m/z: 356 [M+H]⁺.

### Example 7. 5-[2-Fluoro-6-hydroxy-4-[(4-hydroxyquinazolin-2-yl)amino]phenyl]-1,1-dioxo-1,2,5-thiadiazolidin-3-one (Compound 8)

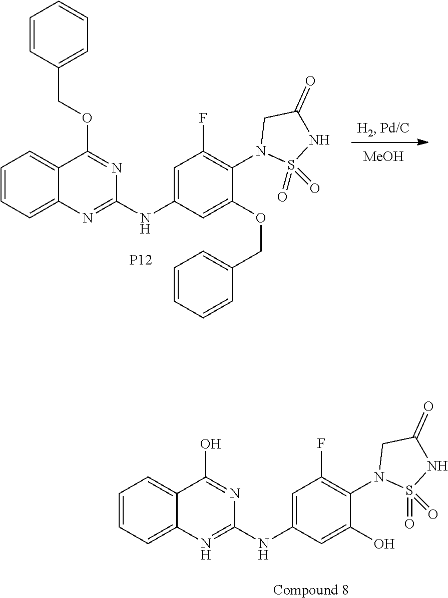

The 5-[2-benzyloxy-4-[(4-benzyloxyquinazolin-2-yl)amino]-6-fluoro-phenyl]-1,1-dioxo-1,2,5-thiadiazolidin-3-one (P12, 0.5 g, 0.85 mmol) was dissolved in methanol (25 mL). Pd/C (24 mg) was added, and the mixture was stirred under H₂ atmosphere for 12 h. The catalyst was removed by filtration and the solution was evaporated to dryness. The residue purified by HPLC (58 mg, 17% yield). ¹H NMR (400 MHz, DMSO-d₆), δ: 10.43 (s, 1H), 8.95 (s, 1H), 7.98 (dd, J=7.9, 1.6 Hz, 1H), 7.69 (m, 1H), 7.46 (d, J=8.1 Hz, 1H), 7.28 (m, 2H), 7.11 (t, J=1.9 Hz, 1H), 4.37 (s, 2H). LCMS (ESI⁺) m/z: 406 [M+H]⁺.

### Example 8. 5-[2-Fluoro-6-hydroxy-4-[(4-hydroxy-6-methoxy-pyrimidin-2-yl)amino]phenyl]-1,1-dioxo-1,2,5-thiadiazolidin-3-one (Compound 9)

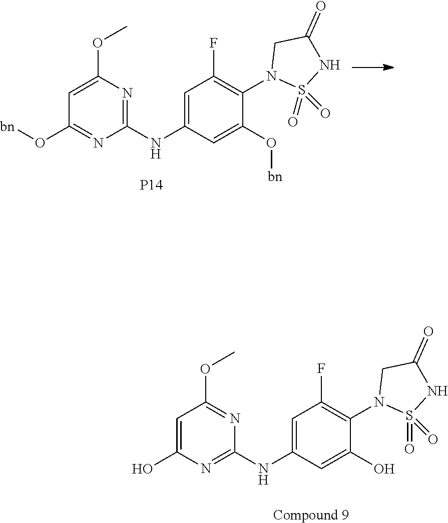

To a solution of 5-[2-benzyloxy-4-[(4-benzyloxy-6-methoxy-pyrimidin-2-yl)amino]-6-fluoro-phenyl]-1,1-dioxo-1,2,5-thiadiazolidin-3-one (P14, 210 mg) in MeOH (20 mL) 10% wet Pd/C (30 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give target compound 9 (26 mg, 18%). ¹H NMR (400 MHz, DMSO-d₆) δ 10.36 (s, 1H), 9.32 (s, 1H), 7.26 (d, J=11.7 Hz, 1H), 7.03 (t, J=1.9 Hz, 1H), 5.30 (s, 1H), 4.36 (s, 2H), 3.83 (s, 3H). LCMS (ESI⁺) m/z: 386 [M+H]⁺.

### Example 9. 5-[2-Fluoro-6-hydroxy-4-[(4-hydroxy-6-methyl-pyrimidin-2-yl)amino]phenyl]-1,1-dioxo-1,2,5-thiadiazolidin-3-one (Compound 10)

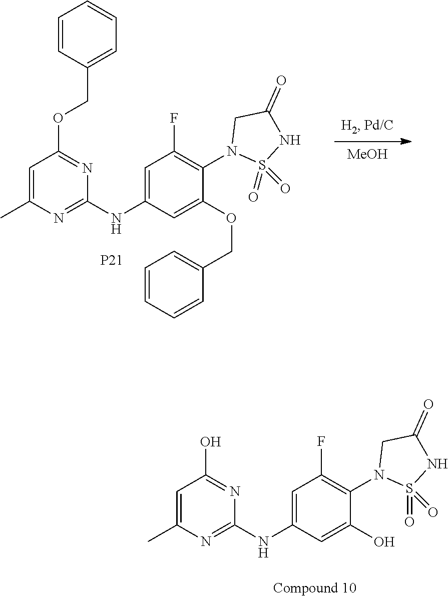

5-[2-Benzyloxy-4-[(4-benzyloxy-6-methyl-pyrimidin-2-yl)amino]-6-fluoro-phenyl]-1,1-dioxo-1,2,5-thiadiazolidin-3-one (P21, 0.15 g, 0.27 mmol) was dissolved in methanol (25 mL). Pd/C (20 mg) was added, and the mixture was stirred under H₂ atmosphere for 12 h. The catalyst was removed by filtration and the solution was evaporated to dryness. The residue purified by HPLC to give the compound 10 (0.084 g, 84% yield). ¹H NMR (400 MHz, DMSO-d₆), δ: 10.32 (s, 1H), 9.23 (s, 1H), 7.28 (d, J=12.5 Hz, 1H), 6.97 (s, 1H), 5.87 (s, 1H), 4.34 (s, 2H), 2.19 (s, 3H). LCMS (ESI⁺) m/z: 370 [M+H]⁺.

### Example 10. 5-[4-[(4-Chloro-6-hydroxy-5-isobutyl-pyrimidin-2-yl)amino]-2-fluoro-6-hydroxy-phenyl]-1,1-dioxo-1,2,5-thiadiazolidin-3-one and 5-[2-fluoro-6-hydroxy-4-[(4-hydroxy-5-isobutyl-pyrimidin-2-yl)amino]phenyl]-1,1-dioxo-1,2,5-thiadiazolidin-3-one (Compounds 42 and 12)

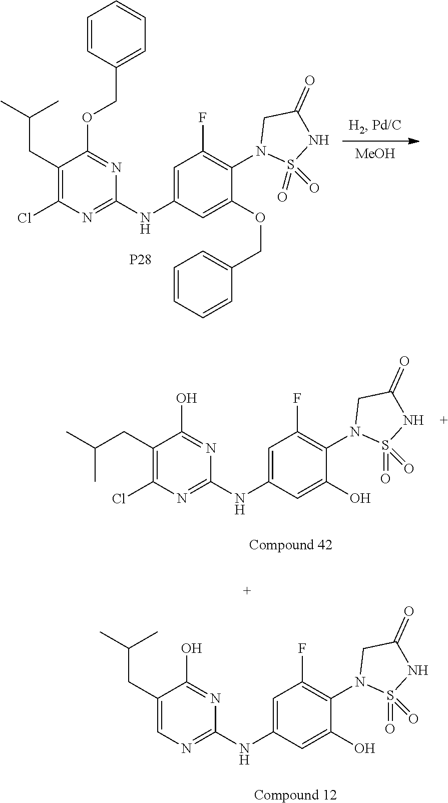

5-[2-benzyloxy-4-[(4-benzyloxy-6-chloro-5-isobutyl-pyrimidin-2-yl)amino]-6-fluoro-phenyl]-1,1-dioxo-1,2,5-thiadiazolidin-3-one (P28, 0.6 g, 0.9 mmol) was dissolved in methanol (25 mL). Pd/C (70 mg) was added, and the mixture was stirred under H₂ atmosphere for 24 h. The catalyst was removed by filtration and the solution was evaporated to dryness. The residue was separated by HPLC to give compound 12 (35 mg, 8%) and compound 42 (0.12 g, 24%). Compound 12: ¹H NMR (400 MHz, DMSO-d₆), δ: 10.34 (s, 1H), 9.18 (s, 1H), 7.61 (s, 1H), 7.15 (dd, J=12.3, 2.4 Hz, 1H), 6.93 (t, J=1.9 Hz, 1H), 4.31 (s, 2H), 2.15 (d, J=7.0 Hz, 2H), 1.83 (m, 1H), 0.85 (d, J=6.6 Hz, 6H); LCMS (ESI⁺) m/z: 412 [M+H]⁺. Compound 42: LCMS (ESI⁺) m/z: 446 [M+H]⁺.

### Example 11. 5-[4-[(4,6-Dihydroxy-5-isobutyl-pyrimidin-2-yl)amino]-2-fluoro-6-hydroxy-phenyl]-1,1-dioxo-1,2,5-thiadiazolidin-3-one (Compound 13)

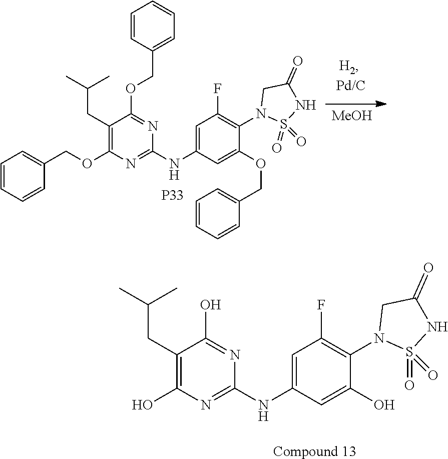

5-[2-Benzyloxy-4-[(4,6-dibenzyloxy-5-isobutyl-pyrimidin-2-yl)amino]-6-fluoro-phenyl]-1,1-dioxo-1,2,5-thiadiazolidin-3-one (P33, 0.16 g, 0.18 mmol) was dissolved in methanol (25 mL). Pd/C (20 mg) was added, and the mixture was stirred under H₂ atmosphere for 24 h. The catalyst was removed by filtration and the solution was evaporated to dryness. The residue purified by HPLC. Yield 0.045 g (56%). ¹H NMR (400 MHz, DMSO-d₆), δ: 10.21 (s, 1H), 8.87 (s, 1H), 7.55 (dd, J=12.8, 2.4 Hz, 1H), 6.59 (s, 1H), 4.31 (s, 2H), 2.11 (d, J=7.2 Hz, 2H), 1.81 (m, 1H), 0.83 (d, J=6.6 Hz, 6H). LCMS (ESI⁺) m/z: 428 [M+H]⁺.

### Example 12. 5-[2-Fluoro-6-hydroxy-4-[(4-hydroxy-6-methyl-5-propyl-pyrimidin-2-yl)amino]phenyl]-1,1-dioxo-1,2,5-thiadiazolidin-3-one (Compound 21)

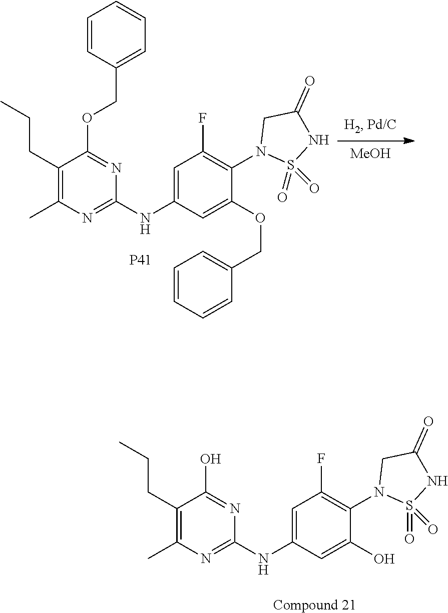

5-[2-benzyloxy-4-[(4-benzyloxy-6-methyl-5-propyl-pyrimidin-2-yl)amino]-6-fluoro-phenyl]-1,1-dioxo-1,2,5-thiadiazolidin-3-one (P41, 0.28 g, 0.47 mmol) was dissolved in methanol (25 mL). Pd/C (30 mg) was added, and the mixture was stirred under H₂ atmosphere for 12 h. The catalyst was removed by filtration and the solution was evaporated to dryness. The residue purified by HPLC to give compound 21 (0.109 g, 57% yield). ¹H NMR (400 MHz, DMSO-d₆), δ: 10.29 (s, 1H), 9.14 (s, 1H), 7.23 (d, J=12.4 Hz, 1H), 6.93 (s, 1H), 4.31 (s, 2H), 2.34 (t, J=7.6 Hz, 2H), 2.23 (s, 3H), 1.42 (m, 2H), 0.89 (t, J=7.3 Hz, 3H). LCMS (ESI⁺) m/z: 411[M+H]⁺.

### Example 13. 5-[2-fluoro-6-hydroxy-4-[(4-hydroxy-5,6,7,8-tetrahydroquinazolin-2-yl)amino]phenyl]-1,1-dioxo-1,2,5-thiadiazolidin-3-one (Compound 39)

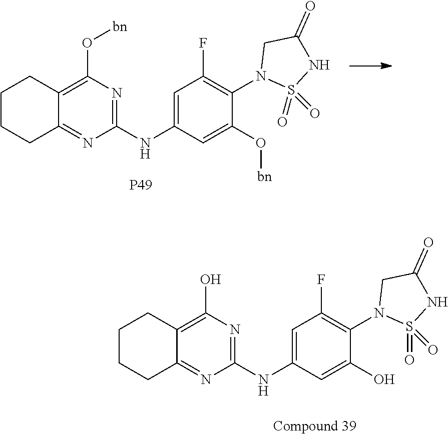

To a solution of 5-[2-benzyloxy-4-[(4-benzyloxy-5,6,7,8-tetrahydroquinazolin-2-yl)amino]-6-fluoro-phenyl]-1,1-dioxo-1,2,5-thiadiazolidin-3-one (P49, 340 mg, 0.58 mmol) in MeOH (20 mL) 10% wet Pd/C (80 mg) was added. The resulting suspension was degassed by 3 vacuum/hydrogen backfills, and then stirred for 12 h under H₂ (1 bar) at rt. After filtration through a small pad of Celite the filtrate was concentrated under reduced pressure, and the residue was purified by preparative HPLC to give 5-[2-fluoro-6-hydroxy-4-[(4-hydroxy-5,6,7,8-tetrahydroquinazolin-2-yl)amino]phenyl]-1,1-dioxo-1,2,5-thiadiazolidin-3-one (Compound 39, 60 mg, 32%). ¹H NMR (400 MHz, DMSO-d₆), δ: 10.26 (s, 1H), 8.99 (s, 1H), 7.20 (dd, J=12.4, 2.4 Hz, 1H), 6.92 (s, 1H), 4.31 (s, 2H), 2.50 (m, 2H), 2.29 (m, 2H), 1.69 (m, 4H). LCMS (ESI⁺) m/z: 411 [M+H]⁺.

### Biological Assays

### Example A. Primary Assay Used to Determine Potency of PTPN2 Enzymatic Activity Inhibition

Compound activity was determined using GST-tagged PTPN2 protein (Cat #31592, ActiveMotif) (SEQ ID NO: 1) in an in vitro enzymatic reaction. The enzymatic reaction was carried out in assay buffer (50 mM HEPES Na salt pH 7.2-7.4, 2 mM EDTA, 100 mM NaCl, 52 ng/μL BSA, and 6 mM DTT). The compounds were dispensed on a 384 well Diamond Well Plate (Axigen, Cat #P-384-120SQ-C-S) using the Biomek FX liquid handling system at 100× solutions of compounds in DMSO. 2×PTPN2 (final concentration 0.004 ng/μL) was prepared in 1× Assay buffer and 25 μL of mixture per well was added into Reaction plate (Optiplate, black, Perkin Elmer, Cat #6007270). Add 25 μL of 1× buffer to Ctrl- (Substrate w/o PTPN2) wells followed by centrifugation at 100 g for 1 min. Next step the Compounds were added to Reaction plate using Biomek station via following steps: 3 μl of 100× compounds (in DMSO) were mixed with 27 μL of Assay Buffer, then 5 μL of this mixture was added to Reaction plate with 25 μL of PTPN2 Mix. Plates were centrifuged for 1 min at 100 g and incubated for another 10 min at rt. Finally, 20 μL of 2.5× Substrate (DiFUMP, Invitrogen™ Cat #D6567) mix was added into appropriate wells of Reaction plate to the final concentration of 2 μM, plate was centrifuged at 100 g for 1 min followed by incubation at rt for 60 min, and the Fluorescence Intensity was measured using a Microplate Reader (ClarioStar Plus, excitation 360 nm, emission 450 nm). The % inhibition was then used to calculate the Kᵢ values. The Kᵢ values are shown in Table A, wherein “A” corresponds to Kᵢ<1.0 nm, “B” corresponds to 1.0 nm≤Kᵢ<5.0 nm, “C” corresponds to 5.0 nm≤Kᵢ<10.0 nm, “D” corresponds to 10.0 nm≤Kᵢ<50.0 nm, and “E” corresponds to 50.0 nm≤Kᵢ<100.0 nm, and “F” corresponds to 100.0 nm≤Kᵢ.

TABLE A
PTPN2 enzymatic activity Inhibition

| Compound | |
|---|---|
| Number | Kᵢ, nM* |
| 2 | C |
| 3 | D |
| 4 | F |
| 5 | E |
| 6 | A |
| 7 | B |
| 8 | A |
| 9 | A |
| 10 | B |
| 12 | A |
| 13 | B |
| 21 | B |
| 39 | A |
| 42 | A |

*Kᵢ: inhibition constant - the concentration required to occupy 50% of the receptor; wherein “A” corresponds to Kᵢ < 1.0 nm, “B” corresponds to 1.0 nm ≤ Kᵢ < 5.0 nm, “C” corresponds to 5.0 nm ≤ Kᵢ < 10.0 nm, “D” corresponds to 10.0 nm ≤ Kᵢ < 50.0 nm, and “E” corresponds to 50.0 nm ≤ Kᵢ < 100.0 nm, and “F” corresponds to 100.0 nm ≤ Kᵢ.

### Example B. Tumor Cells B16F10 Cellular Growth IFNγ-Induced Inhibition Assay

B16F10 mouse melanoma cells (ATCC Cat #CRL-6475) were seeded at a density of 500 cells per well in a 384-well clear bottom plate (Corning Cat #3712, Corning, N.Y.) in 40 μL total volume of DMEM+10% FBS (PanEco Cat #C420, Russia and Sigma Cat #F4135, St. Louis, MO). Cells were allowed to adhere overnight at 37° C., 5% CO₂. On the following day, 250× solutions of compounds in DMSO (Sigma Cat #D2650) were prepared into Cmpnds plate (Diamond Well Plate, Axigen, Cat #P-384-120SQ-C-S) (final concentration of 1×) and DMSO only controls were included. Dilution Plate (Diamond Well Plate (Axigen, Cat #P-384-120SQ-C-S) was prepared by adding 49 μL of culture medium per well: half of the plate with culture medium only, half with culture medium+IFNγ (5 ng/ml). A 1 μL aliquot of 250× compounds (Cmpnds plate) was added to 49 μL of culture medium (Dilution plate), then a 10 μL aliquot of the mixture was transferred to Reaction plate with 40 μL of cells followed by centrifugation at 240 g for 1 min. After 3 days of incubation, 10 μL of CellTiter-Glo (Promega) was added to the cells, plates were centrifuged at 240 g for 1 min, and luminescence signal was measured. For each compound, the percent growth inhibition at every compound dose level was calculated relative to the “DMSO/with IFNγ” control and used to determine the IC₅₀. The IC₅₀ values are shown in Table B, wherein “A” corresponds to IC₅₀<5.0 μM, “B” corresponds to 5.0 μM≤IC₅₀<10.0 μM, “C” corresponds to 10.0 μM≤IC₅₀<50.0 μM, “D” corresponds to 50.0 μM≤IC₅₀<100.0 μM, and “E” corresponds to 100.0 μM≤IC₅₀; and the growth inhibition percentages are shown wherein “*” corresponds to percent growth inhibition<10.0%, “**” corresponds to 10.0%≤percent growth inhibition<50.0%, “***” corresponds to 50.0%≤percent growth inhibition<75.0%, and “****” corresponds to 75.0%≤percent growth inhibition≤100.0%.

TABLE B
Growth Inhibition of B16F10

| | IC₅₀ no | IC₅₀ + | % Growth | % Growth |
|---|---|---|---|---|
| Compound | IFNγ | IFNγ | Inhibition at | Inhibition at |
| Number | (μM) | (μM) | 100 μM no IFNγ | 100 μM + IFNγ |
| 2 | E | A | ** | **** |
| 3 | C | B | ** | *** |
| 4 | E | E | ** | ** |
| 5 | E | E | ** | ** |
| 6 | E | D | * | *** |
| 7 | E | B | ** | **** |
| 8 | E | C | ** | *** |
| 9 | E | B | ** | **** |
| 10 | E | A | ** | **** |
| 12 | E | C | ** | *** |
| 13 | E | C | ** | *** |
| 21 | E | C | ** | *** |
| 39 | E | C | ** | *** |
| 42 | E | C | ** | **** |

### Example C. Pharmacokinetics Studies

Pharmacokinetic (PK) study in mice: Male CD-1 mice obtained from Charles River GmbH (Sulzfeld, Germany) with body weight ranging between 30 g to 40 g were used in the PK studies. Group of 12 mice were given a 2 mg/kg intravenous bolus (IV) dose of test article as a solution in 20% HP-beta-CD, and another group of 9 mice were given a 10 mg/kg oral (PO) dose of test article as a solution or suspension in [10% ethanol, 30% PEG-400 and 60% Phosal-50PG]. Blood samples (˜200 μL per time point) were collected twice from each animal—from orbital sinus and by cardiopuncture at 0.083, 0.25, 0.5, 1, 2, 4, 8 and 24 hours after IV administration, and at 0.5, 1, 2, 4, 8 and 24 hours after PO administration, 3 mice/time point. Blood samples were collected in tubes with Na-EDTA 0.5 M solution (1:10) and centrifuged for 10 min at 10,000 rpm at 2 to 8° C. to harvest plasma. Plasma samples were stored at −80° C. until LC/MS/MS analysis. Concentration in each plasma sample was determined by a non-validated LC/MS/MS. Data is acquired using multiple reaction monitoring (MRM) with specific transitions monitored for each compound. Pharmacokinetic Analysis: PK parameters were calculated by non-compartmental methods as described in Gibaldi and Perrier (Gibaldi and Perrier, 1982) using Phoenix® WinNonlin® version 6.3 (Certara L.P.). Following PO administration, percent bioavailability (Fabs) was determined by dividing the dose normalized mean area under the plasma concentration-time curve, extrapolated to the last time point (AUClast) obtained following PO dose by the mean dose normalized AUC of the animals dosed by IV injection. All PK parameters are presented as mean±standard deviation (SD). PK studies were conducted in mice for exemplary compounds of Formula (I). The percent bioavailability (Fabs) values determined are listed in Table C for certain compounds, together with the dosage, vehicle and form used in the studies.

TABLE C
Oral bioavailability.

| Compound | Fabs, | Dose | | |
|---|---|---|---|---|
| Number | % | (mg/kg) | Vehicle | Form |
| 3 | 12.5 | 10 | 10% Ethanol, 30% PEG- | Solution |
| | | | 400, 60% Phosal-50PG | |
| 7 | 6.1 | 10 | 10% Ethanol, 30% PEG- | Suspension |
| | | | 400, 60% Phosal-50PG | |
| 9 | 10.4 | 10 | 10% Ethanol, 30% PEG- | Solution |
| | | | 400, 60% Phosal-50PG | |
| 10 | 2.7 | 10 | 10% Ethanol, 30% PEG- | Suspension |
| | | | 400, 60% Phosal-50PG | |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (I): 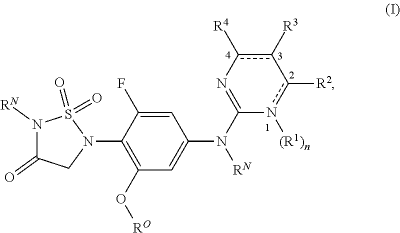
or a pharmaceutically acceptable salt, stereoisomer, solvate, or tautomer thereof, wherein,
each bond [symbol] is independently selected from a single or a double bond;
n is 0 or 1;
provided that when bond 1 [symbol] 2 is a single bond, n is 1 and when bond 1 [symbol] 2 is a double bond, n is 0;
R¹ is selected from hydrogen, deuterium, C₁-C₆ alkyl, C₂-C₆ alkenyl, C₂-C₆ alkynyl, C₁-C₆ alkyl-C(O)—, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
R² is selected from hydrogen, deuterium, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
R³ is selected from hydrogen, deuterium, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
or R² and R³ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵;
R⁴ is selected from hydrogen, deuterium, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁵;
or R³ and R⁴ together with the atoms to which they are attached and any intervening atoms, form a 3-10 membered cycloalkyl, a 6-10 membered aryl, a 3-14 membered heterocycle, or a 5-6 membered heteroaryl, wherein the cycloalkyl, aryl, heterocycle or heteroaryl is optionally substituted with one or more R⁵;
each R^{N} is independently selected from hydrogen, deuterium, C₁-C₆ alkyl, —C(O)C₁-C₆ alkyl, —C(O)OC₁-C₆ alkyl;
R^{O} is selected from hydrogen, deuterium, C₁-C₆ alkyl, —C(O)C₁-C₆ alkyl, —C(O)OC₁-C₆ alkyl, —CH₂-aryl;
each R⁵ is independently selected from deuterium, halogen, —OH, —CN, —NO₂, —NR⁶R⁷, C₁-C₆ alkyl, C₂-C₆ alkenyl, C₂-C₆ alkynyl, C₁-C₆ alkoxy, —C(O)OR⁶, —C(O)NR⁶R⁷, cycloalkyl, —O-cycloalkyl, aryl, heterocyclyl, and heteroaryl, wherein the alkyl, alkenyl, alkynyl, alkoxy, cycloalkyl, aryl, heterocyclyl, or heteroaryl is optionally substituted with one or more R⁸;
R⁶ and R⁷ are each independently selected from hydrogen, C₁-C₆ alkyl, C₂-C₆ alkenyl, C₂-C₆ alkynyl, C₁-C₆ haloalkyl, cycloalkyl, aryl, heterocyclyl, and heteroaryl, wherein the alkyl, alkenyl, alkynyl, cycloalkyl, aryl, heterocyclyl, or heteroaryl is optionally substituted with one or more R⁸;
or R⁶ and R⁷ together with the atoms to which they are attached and any intervening atoms, form a 3-14 membered heterocycle, or a 5-6 membered heteroaryl, wherein the heterocycle or heteroaryl is optionally substituted with one or more R⁸
each R⁸ is independently selected from halogen, OH, CN, NR⁶R⁷, ═NH, NO₂, C₁-C₆ alkyl, C₁-C₆ alkoxy, C₁-C₆ alkyl-C₁-C₆ alkoxy, C₁-C₆ alkyl-NHC₁-C₆ alkyl, cycloalkyl, heterocyclyl, aryl, and heteroaryl;
wherein,
cycloalkyl is mono or polycyclic saturated carbon rings containing 3-18 carbon atoms;
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

2. The compound of claim 1, wherein the compound is of Formula (I-I): 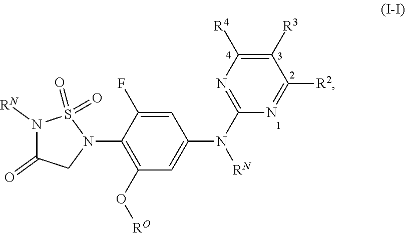
or a pharmaceutically acceptable salt, stereoisomer, solvate, or tautomer thereof.

3. The compound of claim 1, wherein the compound is of Formula (I-II): 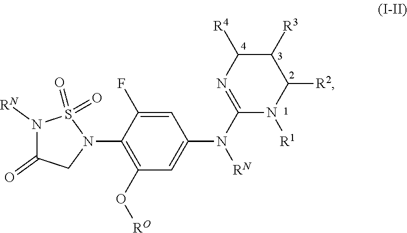
or a pharmaceutically acceptable salt, stereoisomer, solvate, or tautomer thereof.

4. The compound of claim 1, wherein the compound is of Formula (I-I-H) or I-II-H 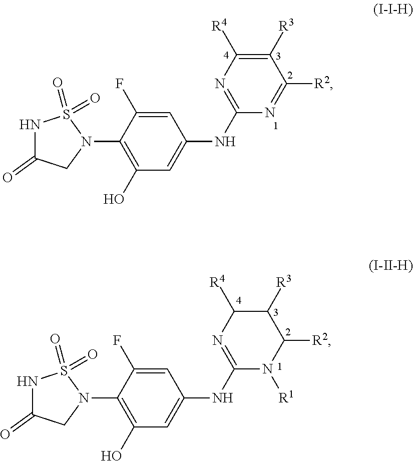
or a pharmaceutically acceptable salt, stereoisomer, solvate, or tautomer thereof.

5. The compound of claim 1, wherein the compound is of Formula (I-I-A), (I-I-B), (I-I-C), (I-I-D), or (I-II-A): 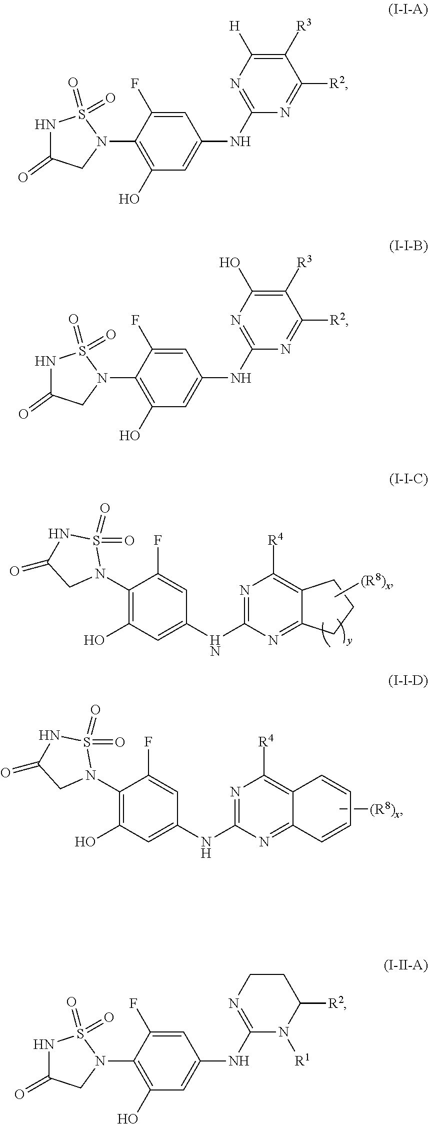
or a pharmaceutically acceptable salt, stereoisomer, solvate, or tautomer thereof wherein y is an integer selected from 0, 1, 2, 3, 4; x is an integer selected from 0, 1, 2, 3, 4; and R⁴ and R⁸ are as defined herein.

6. The compound of any one of claims 1-4, wherein
R¹ is hydrogen;
R² is selected from hydrogen, halogen, —OH, —CN, —NO₂, —CH₃, —OCH₃, —OCH₂CH₃, —OCH₂CH₂CH₃, —OCH₂CH(CH₃)₂, —O(CH₂)₂CH(CH₃)₂, 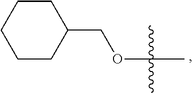 [SMILES: *OCC1CCCCC1]
—NH(CH₃), —N(CH₃)₂, —NHCH₂CH₃, —NH(CH₂)₂CH₃, —N(CH₃)CH₂CH(CH₃)₂, —NH(CH₂)₂CH(CH₃)₂, —N(CH₃)CH₂Ph, 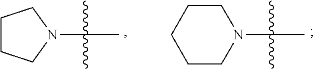 [SMILES: *N1CCCC1 ; *N1CCCCC1]
R³ is selected from hydrogen, halogen, —OH, —CN, —NO₂, —CH₃, —C₂H₅, —CH₂CH₂CH₃, —CH₂CH(CH₃)₂, —(CH₂)₂CH(CH₃)₂, 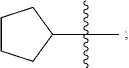 [SMILES: *C1CCCC1]
or R² and R³ together with the atoms to which they are attached and any intervening atoms, form 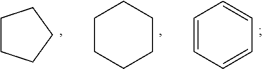
R⁴ is selected from hydrogen, deuterium, halogen, —OH, —CN, —NO₂, —CH₃, —OCH₃, —OCH₂CH₃, —OCH₂CH₂CH₃, —OCH₂CH(CH₃)₂, —O(CH₂)₂CH(CH₃)₂, 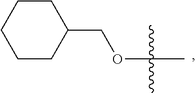 [SMILES: *OCC1CCCCC1]
—NH(CH₃), —N(CH₃)₂, —NHCH₂CH₃, —NH(CH₂)₂CH₃, —N(CH₃)CH₂CH(CH₃)₂, —NH(CH₂)₂CH(CH₃)₂, —N(CH₃)CH₂Ph, 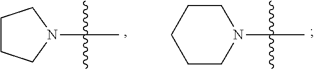 [SMILES: *N1CCCC1 ; *N1CCCCC1]
or R³ and R⁴ together with the atoms to which they are attached and any intervening atoms, form 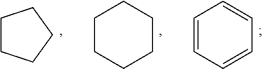
each R^{N} is H;
R^{O} is H;
or a pharmaceutically acceptable salt, stereoisomer, solvate, or tautomer thereof.

7. A compound selected from:

| # | Structure | IUPAC Name |
|---|---|---|
| 1 | 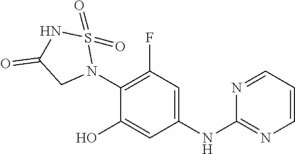 [SMILES: O=C1CN(c2c(O)cc(Nc3ncccn3)cc2F)S(=O)(=O)N1] | 5-[2-fluoro-6-hydroxy-4-(pyrimidin-2- ylamino)phenyl]-1,1-dioxo-1,2,5- thiadiazolidin-3-one |
| 2 | 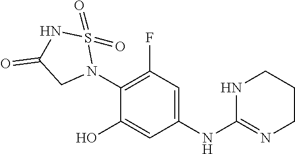 [SMILES: O=C1CN(c2c(O)cc(NC3=NCCCN3)cc2F)S(=O)(=O)N1] | 5-[2-fluoro-6-hydroxy-4-(1,4,5,6- tetrahydropyrimidin-2- ylamino)phenyl]-1,1-dioxo-1,2,5- thiadiazolidin-3-one |
| 3 | 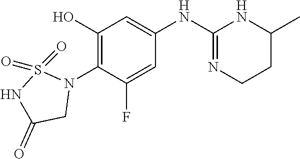 [SMILES: CC1CCN=C(Nc2cc(O)c(N3CC(=O)NS3(=O)=O)c(F)c2)N1] | 5-[2-fluoro-6-hydroxy-4-[(6-methyl- 1,4,5,6-tetrahydropyrimidin-2- yl)amino]phenyl]-1,1-dioxo-1,2,5- thiadiazolidin-3-one |
| 4 | 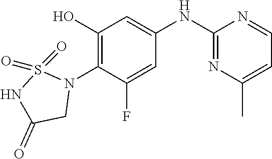 [SMILES: Cc1ccnc(Nc2cc(O)c(N3CC(=O)NS3(=O)=O)c(F)c2)n1] | 5-[2-fluoro-6-hydroxy-4-[(4- methylpyrimidin-2-yl)amino]phenyl]- 1,1-dioxo-1,2,5-thiadiazolidin-3-one |
| 5 | 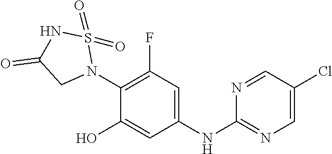 [SMILES: O=C1CN(c2c(O)cc(Nc3ncc(Cl)cn3)cc2F)S(=O)(=O)N1] | 5-[4-[(5-chloropyrimidin-2-yl)amino]- 2-fluoro-6-hydroxy-phenyl]-1,1- dioxo-1,2,5-thiadiazolidin-3-one |
| 6 | 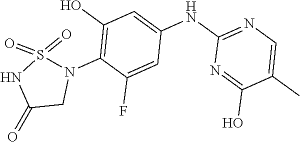 [SMILES: Cc1cnc(Nc2cc(O)c(N3CC(=O)NS3(=O)=O)c(F)c2)nc1O] | 5-[2-fluoro-6-hydroxy-4-[(4-hydroxy- 5-methyl-pyrimidin-2- yl)amino]phenyl]-1,1-dioxo-1,2,5- thiadiazolidin-3-one |
| 7 | 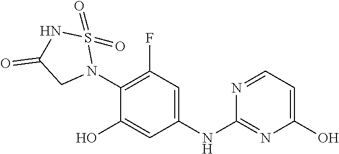 [SMILES: O=C1CN(c2c(O)cc(Nc3nccc(O)n3)cc2F)S(=O)(=O)N1] | 5-[2-fluoro-6-hydroxy-4-[(4- hydroxypyrimidin-2- yl)amino]phenyl]-1,1-dioxo-1,2,5- thiadiazolidin-3-one |
| 8 | 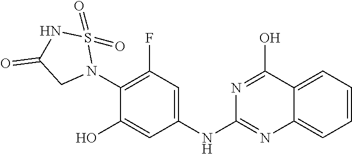 [SMILES: O=C1CN(c2c(O)cc(Nc3nc(O)c4ccccc4n3)cc2F)S(=O)(=O)N1] | 5-[2-fluoro-6-hydroxy-4-[(4- hydroxyquinazolin-2- yl)amino]phenyl]-1,1-dioxo-1,2,5- thiadiazolidin-3-one |
| 9 | 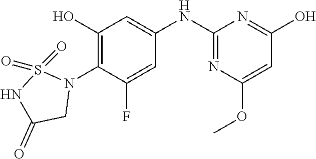 [SMILES: COc1cc(O)nc(Nc2cc(O)c(N3CC(=O)NS3(=O)=O)c(F)c2)n1] | 5-[2-fluoro-6-hydroxy-4-[(4-hydroxy- 6-methoxy-pyrimidin-2- yl)amino]phenyl]-1,1-dioxo-1,2,5- thiadiazolidin-3-one |
| 10 | 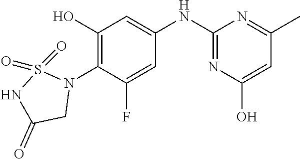 [SMILES: Cc1cc(O)nc(Nc2cc(O)c(N3CC(=O)NS3(=O)=O)c(F)c2)n1] | 5-[2-fluoro-6-hydroxy-4-[(4-hydroxy- 6-methyl-pyrimidin-2- yl)amino]phenyl]-1,1-dioxo-1,2,5- thiadiazolidin-3-one |
| 11 | 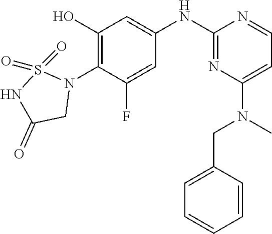 [SMILES: CN(Cc1ccccc1)c1ccnc(Nc2cc(O)c(N3CC(=O)NS3(=O)=O)c(F)c2)n1] | 5-[4-[[4- [benzyl(methyl)amino]pyrimidin-2- yl]amino]-2-fluoro-6-hydroxy- phenyl]-1,1-dioxo-1,2,5- thiadiazolidin-3-one |
| 12 | 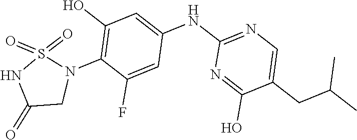 [SMILES: CC(C)Cc1cnc(Nc2cc(O)c(N3CC(=O)NS3(=O)=O)c(F)c2)nc1O] | 5-[2-fluoro-6-hydroxy-4-[(4-hydroxy- 5-isobutyl-pyrimidin-2- yl)amino]phenyl]-1,1-dioxo-1,2,5- thiadiazolidin-3-one |
| 13 | 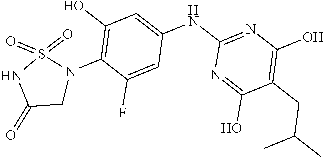 [SMILES: CC(C)Cc1c(O)nc(Nc2cc(O)c(N3CC(=O)NS3(=O)=O)c(F)c2)nc1O] | 5-[4-[(4,6-dihydroxy-5-isobutyl- pyrimidin-2-yl)amino]-2-fluoro-6- hydroxy-phenyl]-1,1-dioxo-1,2,5- thiadiazolidin-3-one |
| 14 | 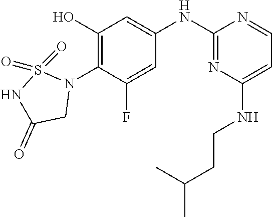 [SMILES: CC(C)CCNc1ccnc(Nc2cc(O)c(N3CC(=O)NS3(=O)=O)c(F)c2)n1] | 5-[2-fluoro-6-hydroxy-4-[[4- (isopentylamino)pyrimidin-2- yl]amino]phenyl]-1,1-dioxo-1,2,5- thiadiazolidin-3-one |
| 15 | 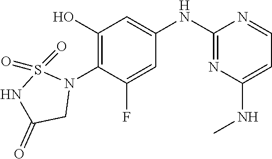 [SMILES: CNc1ccnc(Nc2cc(O)c(N3CC(=O)NS3(=O)=O)c(F)c2)n1] | 5-[2-fluoro-6-hydroxy-4-[[4- (methylamino)pyrimidin-2- ylamino]phenyl]-1,1-dioxo-1,2,5- thiadiazolidin-3-one |
| 16 | 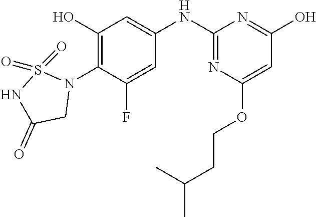 [SMILES: CC(C)CCOc1cc(O)nc(Nc2cc(O)c(N3CC(=O)NS3(=O)=O)c(F)c2)n1] | 5-[2-fluoro-6-hydroxy-4-[(4-hydroxy- 6-isopentyloxy-pyrimidin-2- yl)amino]phenyl]-1,1-dioxo-1,2,5- thiadiazolidin-3-one |
| 17 | 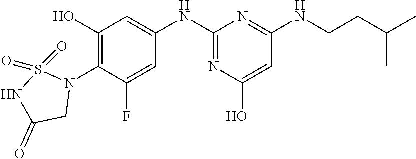 [SMILES: CC(C)CCNc1cc(O)nc(Nc2cc(O)c(N3CC(=O)NS3(=O)=O)c(F)c2)n1] | 5-[2-fluoro-6-hydroxy-4-[[4-hydroxy- 6-(isopentylamino)pyrimidin-2- yl]amino]phenyl]-1,1-dioxo-1,2,5- thiadiazolidin-3-one |
| 18 | 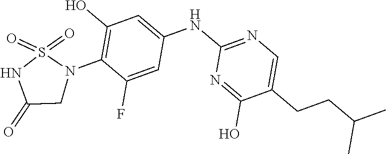 [SMILES: CC(C)CCc1cnc(Nc2cc(O)c(N3CC(=O)NS3(=O)=O)c(F)c2)nc1O] | 5-[2-fluoro-6-hydroxy-4-[(4-hydroxy- 5-isopentyl-pyrimidin-2- yl)amino]phenyl]-1,1-dioxo-1,2,5- thiadiazolidin-3-one |
| 19 | 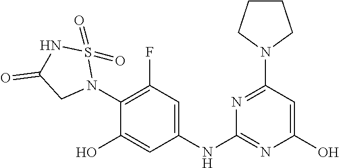 [SMILES: O=C1CN(c2c(O)cc(Nc3nc(O)cc(N4CCCC4)n3)cc2F)S(=O)(=O)N1] | 5-[2-fluoro-6-hydroxy-4-[(4-hydroxy- 6-pyrrolidin-1-yl-pyrimidin-2- yl)amino]phenyl]-1,1-dioxo-1,2,5- thiadiazolidin-3-one |
| 20 | 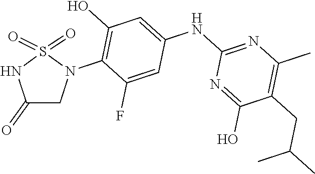 [SMILES: Cc1nc(Nc2cc(O)c(N3CC(=O)NS3(=O)=O)c(F)c2)nc(O)c1CC(C)C] | 5-[2-fluoro-6-hydroxy-4-[(4-hydroxy- 5-isobutyl-6-methyl-pyrimidin-2- yl)amino]phenyl]-1,1-dioxo-1,2,5- thiadiazolidin-3-one |
| 21 | 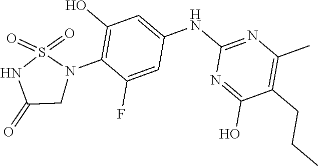 [SMILES: CCCc1c(C)nc(Nc2cc(O)c(N3CC(=O)NS3(=O)=O)c(F)c2)nc1O] | 5-[2-fluoro-6-hydroxy-4-[(4-hydroxy- 6-methyl-5-propyl-pyrimidin-2- yl)amino]phenyl]-1,1-dioxo-1,2,5- thiadiazolidin-3-one |
| 22 | 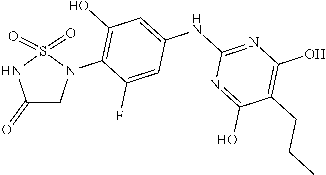 [SMILES: CCCc1c(O)nc(Nc2cc(O)c(N3CC(=O)NS3(=O)=O)c(F)c2)nc1O] | 5-[4-[(4,6-dihydroxy-5-propyl- pyrimidin-2-yl)amino]-2-fluoro-6- hydroxy-phenyl]-1,1-dioxo-1,2,5- thiadiazolidin-3-one |
| 23 | 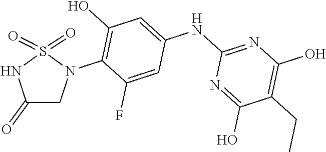 [SMILES: CCc1c(O)nc(Nc2cc(O)c(N3CC(=O)NS3(=O)=O)c(F)c2)nc1O] | 5-[4-[(5-ethyl-4,6-dihydroxy- pyrimidin-2-yl)amino]-2-fluoro-6- hydroxy-phenyl]-1,1-dioxo-1,2,5- thiadiazolidin-3-one |
| 24 | 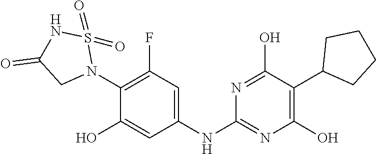 [SMILES: O=C1CN(c2c(O)cc(Nc3nc(O)c(C4CCCC4)c(O)n3)cc2F)S(=O)(=O)N1] | 5-[4-[(5-cyclopentyl-4,6-dihydroxy- pyrimidin-2-yl)amino]-2-fluoro-6- hydroxy-phenyl]-1,1-dioxo-1,2,5- thiadiazolidin-3-one |
| 25 | 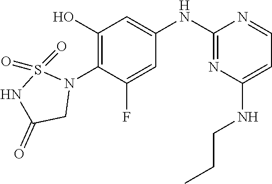 [SMILES: CCCNc1ccnc(Nc2cc(O)c(N3CC(=O)NS3(=O)=O)c(F)c2)n1] | 5-[2-fluoro-6-hydroxy-4-[[4- (propylamino)pyrimidin-2- yl]amino]phenyl]-1,1-dioxo-1,2,5- thiadiazolidin-3-one |
| 26 | 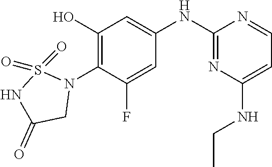 [SMILES: CCNc1ccnc(Nc2cc(O)c(N3CC(=O)NS3(=O)=O)c(F)c2)n1] | 5-[4-[[4-(ethylamino)pyrimidin-2- yl]amino]-2-fluoro-6-hydroxy- phenyl]-1,1-dioxo-1,2,5- thiadiazolidin-3-one |
| 27 | 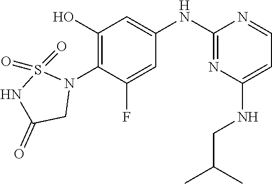 [SMILES: CC(C)CNc1ccnc(Nc2cc(O)c(N3CC(=O)NS3(=O)=O)c(F)c2)n1] | 5-[2-fluoro-6-hydroxy-4-[[4- (isobutylamino)pyrimidin-2- yl]amino]phenyl]-1,1-dioxo-1,2,5- thiadiazolidin-3-one |
| 28 | 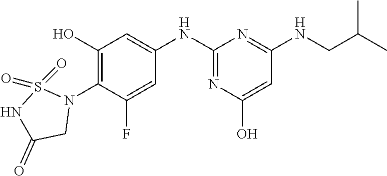 [SMILES: CC(C)CNc1cc(O)nc(Nc2cc(O)c(N3CC(=O)NS3(=O)=O)c(F)c2)n1] | 5-[2-fluoro-6-hydroxy-4-[4-hydroxy- 6-(isobutylamino)pyrimidin-2- yl]amino]phenyl]-1,1-dioxo-1,2,5- thiadiazolidin-3-one |
| 29 | 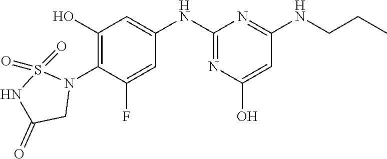 [SMILES: CCCNc1cc(O)nc(Nc2cc(O)c(N3CC(=O)NS3(=O)=O)c(F)c2)n1] | 5-[2-fluoro-6-hydroxy-4-[[4-hydroxy- 6-(propylamino)pyrimidin-2- yl]amino]phenyl]-1,1-dioxo-1,2,5- thiadiazolidin-3-one |
| 30 | 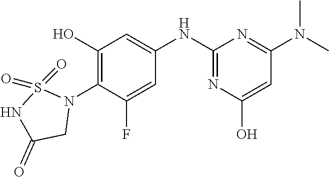 [SMILES: CN(C)c1cc(O)nc(Nc2cc(O)c(N3CC(=O)NS3(=O)=O)c(F)c2)n1] | 5-[4-[[4-(dimethylamino)-6-hydroxy- pyrimidin-2-yl]amino]-2-fluoro-6- hydroxy-phenyl]-1,1-dioxo-1,2,5- thiadiazolidin-3-one |
| 31 | 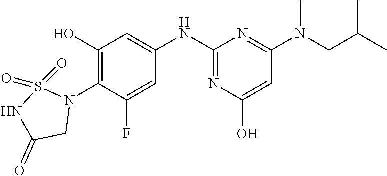 [SMILES: CC(C)CN(C)c1cc(O)nc(Nc2cc(O)c(N3CC(=O)NS3(=O)=O)c(F)c2)n1] | 5-[2-fluoro-6-hydroxy-4-[4-hydroxy- 6-[isobutyl(methyl)amino]pyrimidin- 2-yl]amino]phenyl]-1,1-dioxo-1,2,5- thiadiazolidin-3-one |
| 32 | 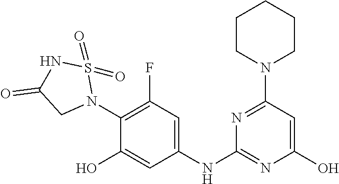 [SMILES: O=C1CN(c2c(O)cc(Nc3nc(O)cc(N4CCCCC4)n3)cc2F)S(=O)(=O)N1] | 5-[2-fluoro-6-hydroxy-4-[4-hydroxy- 6-(1-piperidyl)pyrimidin-2- yl]amino]phenyl]-1,1-dioxo-1,2,5- thiadiazolidin-3-one |
| 33 | 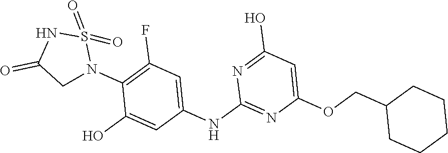 [SMILES: O=C1CN(c2c(O)cc(Nc3nc(O)cc(OCC4CCCCC4)n3)cc2F)S(=O)(=O)N1] | 5-[4-[4-(cyclohexylmethoxy)-6- hydroxy-pyrimidin-2-yl]amino]-2- fluoro-6-hydroxy-phenyl]-1,1-dioxo- 1,2,5-thiadiazolidin-3-one |
| 34 | 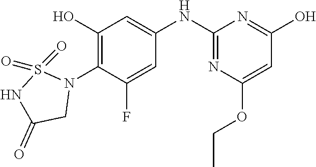 [SMILES: CCOc1cc(O)nc(Nc2cc(O)c(N3CC(=O)NS3(=O)=O)c(F)c2)n1] | 5-[4-[(4-ethoxy-6-hydroxy-pyrimidin- 2-yl)amino]-2-fluoro-6-hydroxy- phenyl]-1,1-dioxo-1,2,5- thiadiazolidin-3-one |
| 35 | 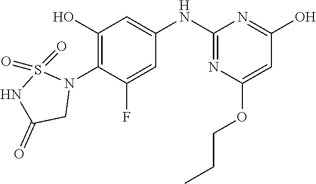 [SMILES: CCCOc1cc(O)nc(Nc2cc(O)c(N3CC(=O)NS3(=O)=O)c(F)c2)n1] | 5-[2-fluoro-6-hydroxy-4-[(4-hydroxy- 6-propoxy-pyrimidin-2- yl)amino]phenyl]-1,1-dioxo-1,2,5- thiadiazolidin-3-one |
| 36 | 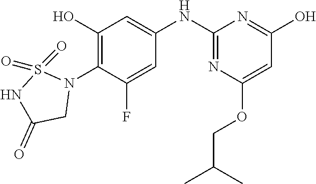 [SMILES: CC(C)COc1cc(O)nc(Nc2cc(O)c(N3CC(=O)NS3(=O)=O)c(F)c2)n1] | 5-[2-fluoro-6-hydroxy-4-[(4-hydroxy- 6-isobutoxy-pyrimidin-2- yl)amino]phenyl]-1,1-dioxo-1,2,5- thiadiazolidin-3-one |
| 37 | 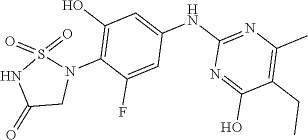 [SMILES: CCc1c(C)nc(Nc2cc(O)c(N3CC(=O)NS3(=O)=O)c(F)c2)nc1O] | 5-[4-[(5-ethyl-4-hydroxy-6-methyl- pyrimidin-2-yl)amino]-2-fluoro-6- hydroxy-phenyl]-1,1-dioxo-1,2,5- thiadiazolidin-3-one |
| 38 | 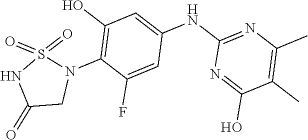 [SMILES: Cc1nc(Nc2cc(O)c(N3CC(=O)NS3(=O)=O)c(F)c2)nc(O)c1C] | 5-[2-fluoro-6-hydroxy-4-[(4-hydroxy- 5,6-dimethyl-pyrimidin-2- yl)amino]phenyl]-1,1-dioxo-1,2,5- thiadiazolidin-3-one |
| 39 | 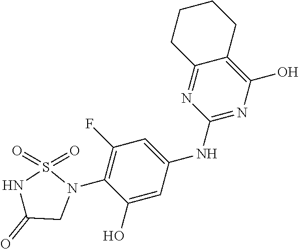 [SMILES: O=C1CN(c2c(O)cc(Nc3nc(O)c4c(n3)CCCC4)cc2F)S(=O)(=O)N1] | 5-[2-fluoro-6-hydroxy-4-[(4-hydroxy- 5,6,7,8-tetrahydroquinazolin-2- yl)amino]phenyl]-1,1-dioxo-1,2,5- thiadiazolidin-3-one |
| 40 | 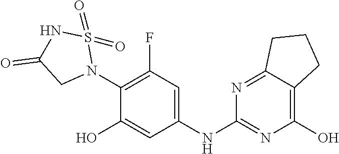 [SMILES: O=C1CN(c2c(O)cc(Nc3nc(O)c4c(n3)CCC4)cc2F)S(=O)(=O)N1] | 5-[2-fluoro-6-hydroxy-4-[(4-hydroxy- 6,7-dihydro-5H- cyclopenta[d]pyrimidin-2- yl)amino]phenyl]-1,1-dioxo-1,2,5- thiadiazolidin-3-one |
| 41 | 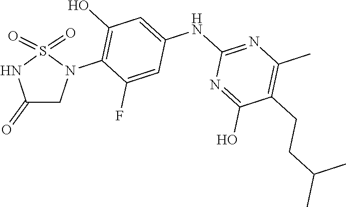 [SMILES: Cc1nc(Nc2cc(O)c(N3CC(=O)NS3(=O)=O)c(F)c2)nc(O)c1CCC(C)C] | 5-[2-fluoro-6-hydroxy-4-[(4-hydroxy- 5-isopentyl-6-methyl-pyrimidin-2- yl)amino]phenyl]-1,1-dioxo-1,2,5- thiadiazolidin-3-one |
| 42 | 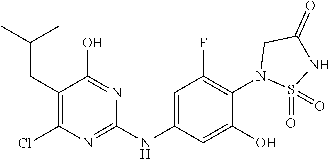 [SMILES: CC(C)Cc1c(O)nc(Nc2cc(O)c(N3CC(=O)NS3(=O)=O)c(F)c2)nc1Cl] | 5-[4-[(4-chloro-6-hydroxy-5-isobutyl- pyrimidin-2-yl)amino]-2-fluoro-6- hydroxy-phenyl]-1,1-dioxo-1,2,5- thiadiazolidin-3-one |

or a pharmaceutically acceptable salt, stereoisomer, solvate, or tautomer thereof.

8. A pharmaceutical composition comprising the compound or pharmaceutically acceptable salt, solvate, hydrate, stereoisomer, or tautomer thereof of claim 1, and a pharmaceutically acceptable carrier.

9. The pharmaceutical composition of claim 8, further comprising an additional pharmaceutically active agent.

10. A method of inhibiting of PTPN1/PTPN2, comprising of administering to a subject a compound of claim 1.

11. A method of treating a disease or disorder associated with PTPN1/PTPN2, comprising of administering to a subject a compound of claim 1.

12. A method of treating disease or disorder selected from cancer, rheumatic disease, inflammatory disease, immune disease, metabolic disease or disorder, infectious disease, neurodegenerative disease, genetic disorder, cardiological disease comprising of administering to a subject in need of a treatment a compound of claim 1.

13. The method of claim 12, wherein rheumatic disease is selected from oligoarticular juvenile idiopathic arthritis; rheumatoid factor-negative polyarticular juvenile idiopathic arthritis.

14. The method of claim 12, wherein inflammatory disease is selected from inflammatory bowel disease, inflammatory bowel disease 20 (IBD20), inflammatory bowel disease 1 (IBD1), Crohn's disease.

15. The method of claim 12, wherein immune disease is selected from immunodeficiency 31c (IMD31C), celiac disease 1 (CELIAC1).

16. The method of claim 12, wherein cancer is selected from T-cell acute lymphoblastic leukemia, ovarian cancer (OC), primary mediastinal B-cell lymphoma, bladder cancer, bone cancer, brain cancer, breast cancer, cardiac cancer, cervical cancer, colon cancer, colorectal cancer, esophageal cancer, fibrosarcoma, gastric cancer, gastrointestinal cancer, head, spine and neck cancer, Kaposi's sarcoma, kidney cancer, leukemia, liver cancer, lymphoma, melanoma, multiple myeloma, pancreatic cancer, penile cancer, testicular germ cell cancer, thymoma carcinoma, thymic carcinoma, lung cancer, ovarian cancer, prostate cancer, marginal zone lymphoma (MZL), follicular lymphoma (FL), diffuse large B-cell lymphoma (DLBCL), pancreatic adenocarcinoma, and chronic lymphocytic leukemia/small lymphocytic lymphoma (CLL/SLL).

17. The method of claim 12, wherein metabolic disease or disorder is selected from obesity—body mass index quantitative trait locus 11 (BMIQ11); diabetes mellitus; type 2 diabetes mellitus (T2D); leptin deficiency or dysfunction; overnutrition.

18. The method of claim 12, wherein infectious disease is selected from bubonic plague.

19. The method of claim 12, wherein genetic disorder is selected from Noonan syndrome, Noonan syndrome with multiple lentigines, RASopathy.

20. The method of claim 12, wherein cardiological disease is selected from Essential Hypertension.

21. The method of any one of claims 10-20, wherein the subject is a mammal.

22. The method of claim 21, wherein the subject is a human.
